(19) United States
(12) Patent Application Publication
(10) Pub. No.: US 2025/0213694 A1
(43) Pub. Date: Jul. 3, 2025

(54) ENHANCED CHIMERIC ANTIGEN RECEPTOR AND USE THEREOF

(71) Applicant: SHANGHAI SIMNOVA BIOTECHNOLOGY CO., LTD., Shanghai (CN)

(72) Inventors: Cuiqing YANG, Shanghai (CN); Yifang WANG, Shanghai (CN); Fuwei JIANG, Shanghai (CN); Chao WANG, Shanghai (CN); Qingyang WANG, Shanghai (CN); Zhuoxiao CAO, Shanghai (CN)

(21) Appl. No.: 18/852,959

(22) PCT Filed: Mar. 31, 2023
(86) PCT No.: PCT/CN2023/085490

§ 371 Date: Sep. 30, 2024

(30) Foreign Application Priority Data
Apr. 3, 2022 (CN) 202210347351.4
Jul. 22, 2022 (CN) 202210871138.3
Mar. 24, 2023 (CN) 202310296244.8

(51) Int. Cl. A61K 40/42; A61K 40/15; A61K 40/31; A61P 35/00; C07K 14/725; C07K 16/28

(52) U.S. Cl. CPC A61K 40/4215; A61K 40/15; A61K 40/31; A61K 40/4202; A61P 35/00; C07K 14/7051; C07K 16/28; C07K 16/2878; C07K 2317/31; C07K 2317/569

(57) ABSTRACT

The present invention relates to an enhanced chimeric antigen receptor and use thereof, including a nucleic acid molecule for encoding a chimeric antigen receptor containing an NKG2D transmembrane region, a corresponding chimeric antigen receptor, a carrier, an immune effector cell, a preparation method and a product thereof, a pharmaceutical composition, pharmaceutical use, and a tumor or cancer treatment method. The present invention has the advantages of high CAR expression efficiency, rapid NK cell proliferation, strong killing ability against tumor cells, high specificity, long duration of killing effect, and the like.

The present invention claims the priority to patent application No. 202210347351.4 entitled “ENHANCED CHIMERIC ANTIGEN RECEPTOR AND USE THEREOF”, filed with China National Intellectual Property Administration on Apr. 3, 2022; Patent Application Ser. No. 202210871138.3 entitled “CHIMERIC ANTIGEN RECEPTOR TARGETING GPRC5D AND/OR BCMA, AND USE THEREOF”, filed with China National Intellectual Property Administration on Jul. 22, 2022; and patent application No. 202310296244.8 entitled “ENHANCED CHIMERIC ANTIGEN RECEPTOR AND USE THEREOF”, filed with China National Intellectual Property Administration on Mar. 24, 2023, which are incorporated herein by reference in their entireties.

### TECHNICAL FIELD

The present disclosure relates to the field of biotechnologies, and particularly, to an enhanced chimeric antigen receptor and use thereof.

### BACKGROUND

Natural killer cells (NK cells) are a group of MHC-independent lymphocytes with a potent killing effect on tumor cells. They recognize tumor cells mainly depending on the mutual cross-regulation of activating receptors and inhibitory receptors on their surface. Upon the recognition of tumor cells, NK cells kill tumor cells through a variety of pathways, such as the release of killing mediators perforin and granzymes to induce the apoptosis of target cells, the expression of membrane TNF family molecules to induce the apoptosis of target cells, and antibody-dependent cytotoxicity. Allogeneic NK cell transplantation seldom induces graft-versus-host disease (GVHD) or CRS, and can be used as a completely “off-the-shelf” product. However, due to the declines in the quantity and quality of NK cells in patients with tumors and the tumor escape mechanism, the anti-tumor functionality of in vivo has not been fully exerted. The modification of NK cells by chimeric antigen receptors (CARs) is expected to enhance their ability to target and kill tumor cells and their potential in the development of effector cells with potent anti-tumor effects.

The structure of a functional CAR expressed on NK cells mainly comprises: an extracellular domain, a transmembrane region, and an intracellular signaling domain. The selection of the corresponding elements in the CAR structure, e.g., the selection of the transmembrane region and the intracellular activation signaling domain, may affect the activity of the CAR-NK cells. Currently, most CAR-NK products still follow the CAR structure commonly used in conventional CAR-T cell therapies, with a CD28 or 4-1BB intracellular co-activation signaling domain. Since the signaling pathways of NK cell activation and T cell activation are different, the CAR structure commonly used in conventional CAR-T cell therapies may not function optimally in CAR-NK.

### SUMMARY

The present disclosure designs a variety of combinations of CAR structures comprising different transmembrane regions and intracellular co-stimulatory domains according to the unique signaling pathway of NK cell activation, and through screening and verification in in vitro and in vivo studies, obtains the CAR structure most suitable for CAR-NK cells to exert their activity.

Furthermore, the present disclosure also optimizes the transmembrane region of CAR-NK. The transmembrane (TM) regions commonly used in CAR-NK come from CD8 and CD28, and may also come from others such as NKG2D. For example, CN110684117B and CN112210016A disclose chimeric antigen receptor-modified NK cells in which the transmembrane region of the chimeric antigen receptor is derived from NKG2D TM. NKG2D is an NK cell activator, and the NKG2D TM region is beneficial to enhancing CAR-NK cell activation and killing effects, but the current CAR-NK structures lack a fine design for the NKG2D TM region. The present disclosure shows that optimizing the NKG2D TM region is of great importance for further improving the function of CAR-NK.

B cell maturation antigen (BCMA), a member of the tumor necrosis factor receptor superfamily, is expressed primarily on the surface of terminally differentiated B cells. B cell activating factor (BAFF) and proliferation-inducing ligand (APRIL) are the major ligands of BCMA, which transduce cellular stimulatory signals by interacting with BCMA to activate the TRAF-dependent NF-κB and JNK pathways and promote the proliferation and survival of B cells. It has been reported that the expression of BCMA is associated with a variety of cancers, autoimmune disorders, and infectious diseases. Cancers with increased BCMA expression include hematological cancers, such as multiple myeloma, Hodgkin's lymphoma, non-Hodgkin's lymphoma, various leukemias, and glioblastoma. BCMA is an ideal therapeutic target.

ROR1 is a transmembrane receptor tyrosine kinase protein and a member of the receptor tyrosine kinases (RTKs) family. ROR1 possesses low or no expression in human normal tissues, but is highly expressed in a variety of malignant tumors or tissues, such as chronic lymphocytic leukemia (CLL), breast cancer, ovarian cancer, melanoma, and lung adenocarcinoma. It has been illuminated that ROR1 plays a pivotal role in promoting the growth and metastasis of tumors, inducing drug resistance in tumor cells, inhibiting apoptosis, etc. Therefore, BCMA and ROR1 are ideal targets in the field of tumor treatments, and the development of corresponding chimeric antigen receptors, especially chimeric antigen receptors suitable for NK cells, against BCMA and ROR1 targets is particularly important.

In view of the above, the present disclosure provides a nucleic acid molecule encoding a chimeric antigen receptor comprising an NKG2D transmembrane region, a corresponding chimeric antigen receptor, a vector, an immune effector cell, a preparation method and a product thereof, a pharmaceutical composition, pharmaceutical use, and a method for treating a tumor or cancer.

In a first aspect, the present disclosure provides a nucleic acid molecule, comprising a first nucleic acid sequence encoding a chimeric antigen receptor (CAR), wherein the CAR comprises: an extracellular domain comprising an antigen-binding region, an NKG2D transmembrane region linked to the extracellular domain, and an intracellular domain linked to the transmembrane region; wherein the NKG2D transmembrane region comprises a sequence having at least 80% identity to or at most 9 mutations compared with SEQ ID NO: 48.

In some specific embodiments, the antigen-binding region has at least one of the properties of groups (1)-(4):
(1) the antigen-binding region is selected from an antibody or a fragment thereof or a ligand binding to the antigen, e.g., scFv, VHH, Fab, F(ab)′₂, or ligand;
(2) the antigen-binding region binds to or does not bind to NKG2DL;
(3) the antigen is selected from one or more of the following group: BCMA, GPRC5D, CLDN18.2, ROR1, CD19, CD33, CD5, CD70, HER2, IL13Rα2, GCC, or GPC3;
(4) the antigen-binding region binds to at least two different antigens, binds to at least two different epitopes of the same antigen, or binds to two or more identical epitopes of the same antigen.

In some specific embodiments, the antigen-binding region specifically binds to BCMA, and comprises:
(1) a VHH, comprising CDRs 1-3 respectively comprising:
the sequences set forth in SEQ ID NO: 9, SEQ ID NO: 10, and SEQ ID NO: 11; or
the sequences set forth in SEQ ID NO: 12, SEQ ID NO: 13, and SEQ ID NO: 11; or
the sequences set forth in SEQ ID NO: 14, SEQ ID NO: 15, and SEQ ID NO: 16;
(2) a VHH, comprising a sequence having at least 80% identity to or at most 25 mutations compared with SEQ ID NO: 1 or 3;
(3) a VHH, comprising CDRs 1-3 respectively comprising:
the sequences set forth in SEQ ID NO: 17, SEQ ID NO: 18, and SEQ ID NO: 19; or
the sequences set forth in SEQ ID NO: 17, SEQ ID NO: 25, and SEQ ID NO: 19; or
the sequences set forth in SEQ ID NO: 20, SEQ ID NO: 21, and SEQ ID NO: 19; or
the sequences set forth in SEQ ID NO: 22, SEQ ID NO: 23, and SEQ ID NO: 24;
(4) a sequence having at least 80% identity to or at most 25 mutations compared with SEQ ID NO: 2 or 4; or
(5) VHH1-linker peptide-VHH2, wherein the VHH1 comprises the sequence set forth in group (1) or group (2), and the VHH2 comprises the sequence set forth in group (3) or group (4); or
(6) VHH1-linker peptide-VHH2, wherein the VHH1 comprises the sequence set forth in group (3) or group (4), and the VHH2 comprises the sequence set forth in group (1) or group (2); or
(7) VHH1-linker peptide-VHH2, comprising a sequence having at least 80% identity to or at most 50 mutations compared with SEQ ID NO: 63.

In some specific embodiments, the antigen-binding region specifically binds to ROR1, and comprises:
(1) an antibody or a fragment thereof, comprising HCDRs 1-3 and LCDRs 1-3, wherein the HCDRs 1-3 respectively comprise:
the sequences set forth in SEQ ID NO: 30, SEQ ID NO: 31, and SEQ ID NO: 32; or
the sequences set forth in SEQ ID NO: 33, SEQ ID NO: 34, and SEQ ID NO: 32; or
the sequences set forth in SEQ ID NO: 35, SEQ ID NO: 36, and SEQ ID NO: 37;
and the LCDRs 1-3 respectively comprise:
the sequences set forth in SEQ ID NO: 38, SEQ ID NO: 39, and SEQ ID NO: 40; or
the sequences set forth in SEQ ID NO: 38, SEQ ID NO: 39, and SEQ ID NO: 40; or
the sequences set forth in SEQ ID NO: 41, SEQ ID NO: 42, and SEQ ID NO: 40;
(2) an antibody or a fragment thereof, comprising a heavy chain variable region having at least 80% identity to or at most 25 mutations compared with SEQ ID NO: 26 and a light chain variable region having at least 80% identity to or at most 25 mutations compared with SEQ ID NO: 27;
(3) an scFv, comprising the sequence set forth in group (1) or group (2), and optionally, comprising a light chain variable region having at least 80% identity to or at most 50 mutations compared with SEQ ID NO: 28.

In some specific embodiments, the antigen-binding region specifically binds to GPRC5D, and comprises:
(1) an antibody or a fragment thereof, comprising HCDRs 1-3 and LCDRs 1-3, wherein the HCDRs 1-3 respectively comprise:
the sequences set forth in SEQ ID NO: 87, SEQ ID NO: 88, and SEQ ID NO: 89; or
the sequences set forth in SEQ ID NO: 90, SEQ ID NO: 91, and SEQ ID NO: 92; or
the sequences set forth in SEQ ID NO: 96, SEQ ID NO: 97, and SEQ ID NO: 98;
and the LCDRs 1-3 respectively comprise:
the sequences set forth in SEQ ID NO: 84, SEQ ID NO: 85, and SEQ ID NO: 86; or
the sequences set forth in SEQ ID NO: 93, SEQ ID NO: 94, and SEQ ID NO: 95;
(2) an antibody or a fragment thereof, comprising HCDRs 1-3, wherein the HCDRs 1-3 respectively comprise:
the sequences set forth in SEQ ID NOs: 107-118;
(3) an antibody or a fragment thereof, comprising a heavy chain variable region having at least 80% identity to or at most 25 mutations compared with SEQ ID NO: 78, 80, 83, or 131 and a light chain variable region having at least 80% identity to or at most 25 mutations compared with SEQ ID NO: 77, 79, 81, or 82;
(4) an antibody or a fragment thereof, comprising a heavy chain variable region having at least 80% identity to or at most 25 mutations compared with SEQ ID NOs: 99-106.

In some specific embodiments, the extracellular domain further comprises a hinge region; the hinge region is selected from one or more of the following group: a CD8a hinge region, a 2B4 hinge region, a CD28 hinge region, an IgG1 hinge region, an IgD hinge, an IgG4 hinge region, a GS hinge, a KIR2DS2 hinge, a KIR hinge, an NCR hinge, a SLAMF hinge, a CD16 hinge, a CD64 hinge, or an LY49 hinge; optionally, the hinge region is selected from a CD8a hinge region; e.g., the hinge region comprises a sequence having at least 80% identity to or at most 10 mutations compared with SEQ ID NO: 45.

In some specific embodiments, the extracellular domain further comprises a signal peptide; the signal peptide is selected from one or more of the following group: a CD8a signal peptide, an IgG1 heavy chain signal peptide, and a GM-CSFR2 signal peptide; optionally, the signal peptide is a CD8a signal peptide; e.g., the signal peptide comprises a sequence having at least 80% identity to or at most 5 mutations compared with SEQ ID NO: 43.

In some specific embodiments, the intracellular domain comprises an intracellular signaling domain and/or a co-stimulatory domain, wherein
optionally, the intracellular signaling domain is CD32; e.g., the intracellular signaling domain comprises a sequence having at least 80% identity to or at most 35 mutations compared with SEQ ID NO: 55; and
optionally, the co-stimulatory domain is selected from one or more of the following group: a CD27 co-stimulatory domain, a 4-1BB co-stimulatory domain, an OX40 co-stimulatory domain, a 2B4 co-stimulatory domain, a CD28 co-stimulatory domain, an ICOS co-stimulatory domain, a DAP10 co-stimulatory domain, or a DAP12 co-stimulatory domain; optionally, the co-stimulatory domain is a 2B4 co-stimulatory domain; e.g., the co-stimulatory domain comprises a sequence having at least 80% identity to or at most 25 mutations compared with SEQ ID NO: 53.

In some specific embodiments, the CAR comprises: a CD8α hinge region, an NKG2D transmembrane region, a 2B4 co-stimulatory domain, and a CD3ζ intracellular signaling domain; e.g., comprising a sequence having at least 80% identity to or at most 50 mutations compared with SEQ ID NO: 58.

In some specific embodiments, the nucleic acid molecule further comprises a second nucleic acid sequence encoding IL15, wherein optionally the IL15 is selected from soluble IL15, membrane-binding IL15, or a complex of IL15 with a receptor thereof or a receptor fragment, and optionally the IL15 comprises a sequence having at least 80% identity to or at most 35 mutations compared with SEQ ID NO: 57;
optionally, the first nucleic acid sequence and the second nucleic acid sequence are linked via an IRES or a sequence encoding a self-cleaving peptide; the self-cleaving peptide is selected from a 2A peptide, e.g., P2A, T2A, F2A, or E2A; optionally the self-cleaving peptide is a P2A, e.g., comprising a sequence having at least 80% identity to or at most 5 mutations compared with SEQ ID NO: 56;
optionally, the nucleic acid encodes an amino acid sequence having at least 85% identity to or at most 100 mutations compared with SEQ ID NO: 59.

In some specific embodiments, the nucleic acid molecule comprises a nucleic acid sequence encoding a sequence having at least 80% identity to or at most 150 mutations compared with SEQ ID NO: 65 or SEQ ID NO: 70.

In some specific embodiments, the nucleic acid is a DNA or an RNA, wherein the RNA is preferably an mRNA.

In a second aspect, the present disclosure provides a nucleic acid molecule, comprising a first nucleic acid sequence encoding a CAR targeting BCMA, wherein the CAR comprises an extracellular domain comprising an antigen-binding region, a transmembrane region linked to the extracellular domain, and an intracellular domain linked to the transmembrane region; the antigen-binding region comprises:
(1) a VHH, comprising CDRs 1-3 respectively comprising:
the sequences set forth in SEQ ID NO: 9, SEQ ID NO: 10, and SEQ ID NO: 11; or
the sequences set forth in SEQ ID NO: 12, SEQ ID NO: 13, and SEQ ID NO: 11; or
the sequences set forth in SEQ ID NO: 14, SEQ ID NO: 15, and SEQ ID NO: 16;
(2) a VHH, comprising a sequence having at least 80% identity to or at most 25 mutations compared with SEQ ID NO: 1 or 3;
(3) a VHH, comprising CDRs 1-3 respectively comprising:
the sequences set forth in SEQ ID NO: 17, SEQ ID NO: 18, and SEQ ID NO: 19; or
the sequences set forth in SEQ ID NO: 17, SEQ ID NO: 25, and SEQ ID NO: 19; or
the sequences set forth in SEQ ID NO: 20, SEQ ID NO: 21, and SEQ ID NO: 19; or
the sequences set forth in SEQ ID NO: 22, SEQ ID NO: 23, and SEQ ID NO: 24;
(4) a sequence having at least 80% identity to or at most 25 mutations compared with SEQ ID NO: 2 or 4; or
(5) VHH1-linker peptide-VHH2, wherein the VHH1 comprises the sequence set forth in group (1) or group (2), and the VHH2 comprises the sequence set forth in group (3) or group (4); or
(6) VHH1-linker peptide-VHH2, wherein the VHH1 comprises the sequence set forth in group (3) or group (4), and the VHH2 comprises the sequence set forth in group (1) or group (2); or
(7) VHH1-linker peptide-VHH2, comprising a sequence having at least 80% identity to or at most 50 mutations compared with SEQ ID NO: 63.

In a third aspect, the present disclosure provides a nucleic acid molecule, comprising a first nucleic acid sequence encoding a CAR targeting ROR1, wherein the CAR comprises an extracellular domain comprising an antigen-binding region, a transmembrane region linked to the extracellular domain, and an intracellular domain linked to the transmembrane region; the antigen-binding region comprises:
(1) an antibody or a fragment thereof, comprising HCDRs 1-3 and LCDRs 1-3, wherein the HCDRs 1-3 respectively comprise:
the sequences set forth in SEQ ID NO: 30, SEQ ID NO: 31, and SEQ ID NO: 32; or
the sequences set forth in SEQ ID NO: 33, SEQ ID NO: 34, and SEQ ID NO: 32; or
the sequences set forth in SEQ ID NO: 35, SEQ ID NO: 36, and SEQ ID NO: 37;
and the LCDRs 1-3 respectively comprise:
the sequences set forth in SEQ ID NO: 38, SEQ ID NO: 39, and SEQ ID NO: 40; or
the sequences set forth in SEQ ID NO: 38, SEQ ID NO: 39, and SEQ ID NO: 40; or
the sequences set forth in SEQ ID NO: 41, SEQ ID NO: 42, and SEQ ID NO: 40;
(2) an antibody or a fragment thereof, comprising a heavy chain variable region having at least 80% identity to or at most 25 mutations compared with SEQ ID NO: 26 and a light chain variable region having at least 80% identity to or at most 25 mutations compared with SEQ ID NO: 27;
(3) an scFv, comprising the sequence set forth in group (1) or group (2), and optionally, comprising a light chain variable region having at least 80% identity to or at most 50 mutations compared with SEQ ID NO: 28.

In some specific embodiments, the CAR comprises: the CD8a hinge region, the NKG2D transmembrane region, the 2B4 co-stimulatory domain, and a CD35 intracellular signaling domain; e.g., the CAR comprises a sequence having at least 80% identity to or at most 50 mutations compared with SEQ ID NO: 58.

In some specific embodiments, the nucleic acid molecule further comprises a second nucleic acid sequence encoding IL15, wherein optionally the IL15 is selected from soluble IL15, membrane-binding IL15, or a complex of IL15 with a receptor thereof or a receptor fragment, and optionally the IL15 comprises a sequence having at least 80% identity to or at most 35 mutations compared with SEQ ID NO: 57;
optionally, the first nucleic acid sequence and the second nucleic acid sequence are linked via an IRES or a sequence encoding a self-cleaving peptide; the self-cleaving peptide is selected from a 2A peptide, e.g., P2A, T2A, F2A, or E2A; optionally the self-cleaving peptide is a P2A, e.g., comprising a sequence having at least 80% identity to or at most 5 mutations compared with SEQ ID NO: 56;
optionally, the nucleic acid encodes an amino acid sequence having at least 85% identity to or at most 100 mutations compared with SEQ ID NO: 59.

In some specific embodiments, the nucleic acid molecule comprises a nucleic acid sequence encoding a sequence having at least 80% identity to or at most 150 mutations compared with SEQ ID NO: 65 or SEQ ID NO: 70.

In a fourth aspect, the present disclosure provides a CAR encoded by the nucleic acid described above.

In a fifth aspect, the present disclosure provides a vector comprising the nucleic acid molecule described above.

In a sixth aspect, the present disclosure provides an immune effector cell comprising the nucleic acid molecule, the CAR, or the vector described above.

In some specific embodiments, the immune effector cell is an NK cell, wherein the NK cell is differentiated from an iPSC, or derived from peripheral blood or umbilical cord blood.

In a seventh aspect, the present disclosure provides a method for preparing the immune effector cell described above, comprising: providing an immune effector cell, and introducing the nucleic acid molecule into the immune effector cell.

In an eighth aspect, the present disclosure provides a product prepared by the method described above.

In a ninth aspect, the present disclosure provides a pharmaceutical composition, comprising the nucleic acid molecule, the CAR, the vector, the immune effector cell or the product described above, and a pharmaceutically acceptable carrier.

In a tenth aspect, the present disclosure provides the nucleic acid molecule, the CAR, the vector, the immune effector cell, the product, or the pharmaceutical composition described above for use in the manufacture of a medicament for treating a cancer or tumor, wherein the cancer or tumor is selected from a hematologic tumor or a solid tumor; optionally, the hematological tumor is selected from myeloma, lymphoma, or leukemia, e.g., multiple myeloma, Hodgkin's lymphoma, non-Hodgkin's lymphoma, diffuse large B-cell lymphoma, follicular lymphoma, mantle cell lymphoma, acute myelogenous leukemia (AML), chronic lymphocytic leukemia (CLL), acute lymphocytic leukemia (ALL), chronic myelogenous leukemia (CML), or hairy cell leukemia (HCL); optionally, the solid tumor is selected from lung cancer, breast cancer, colorectal cancer, gastric cancer, pancreatic cancer, liver cancer, skin cancer, bladder cancer, ovarian cancer, uterine cancer, prostate cancer, or adrenal cancer.

In an eleventh aspect, the present disclosure provides the nucleic acid molecule, the CAR, the vector, the immune effector cell, the product, or the pharmaceutical composition described above for use in the treatment of a cancer or tumor, wherein the cancer or tumor is selected from a hematologic tumor or a solid tumor; optionally, the hematological tumor is selected from myeloma, lymphoma, or leukemia, e.g., multiple myeloma, Hodgkin's lymphoma, non-Hodgkin's lymphoma, diffuse large B-cell lymphoma, follicular lymphoma, mantle cell lymphoma, acute myelogenous leukemia (AML), chronic lymphocytic leukemia (CLL), acute lymphocytic leukemia (ALL), chronic myelogenous leukemia (CML), or hairy cell leukemia (HCL); optionally, the solid tumor is selected from lung cancer, breast cancer, colorectal cancer, gastric cancer, pancreatic cancer, liver cancer, skin cancer, bladder cancer, ovarian cancer, uterine cancer, prostate cancer, or adrenal cancer.

In a twelfth aspect, the present disclosure provides a method for treating a cancer or tumor, comprising: administering to a subject in need an effective amount of the nucleic acid molecule, the CAR, the vector, the immune effector cell, the product, or the pharmaceutical composition described above, wherein the cancer or tumor is selected from a hematologic tumor or a solid tumor; optionally, the hematological tumor is selected from myeloma, lymphoma, or leukemia, e.g., multiple myeloma, Hodgkin's lymphoma, non-Hodgkin's lymphoma, diffuse large B-cell lymphoma, follicular lymphoma, mantle cell lymphoma, acute myelogenous leukemia (AML), chronic lymphocytic leukemia (CLL), acute lymphocytic leukemia (ALL), chronic myelogenous leukemia (CML), or hairy cell leukemia (HCL); optionally, the solid tumor is selected from lung cancer, breast cancer, colorectal cancer, gastric cancer, pancreatic cancer, liver cancer, skin cancer, bladder cancer, ovarian cancer, uterine cancer, prostate cancer, or adrenal cancer.

Beneficial effects: The present disclosure provides an optimized CAR for the NKG2D transmembrane region, which has at least one of the following advantages: (1) high CAR expression efficiency after transfection; (2) high proliferation rate of NK cells after transfection; (3) potent killing ability and high specificity against tumor cells; and (4) prolonged of killing effect.

### Definitions

Unless otherwise defined herein, scientific and technical terms used in correlation with the present disclosure shall have the meanings that are commonly understood by those skilled in the art. Furthermore, unless otherwise stated herein, terms used in the singular form herein shall include the plural form, and vice versa. More specifically, as used in this specification and the appended claims, unless otherwise clearly indicated, the singular forms “a”, “an”, and “the” include referents in the plural form.

The terms “include”, “comprise”, and “have” herein are used interchangeably and are intended to indicate the inclusion of a solution, implying that there may be elements other than those listed in the solution. Meanwhile, it should be understood that the descriptions “include”, “comprise”, and “have” as used herein also provide the solution of “consist of . . . ”. Illustratively, the term “a composition, comprising A and B” should be interpreted as the following technical solution: a composition consisting of A and B, and a composition containing other components in addition to A and B, all of which fall within the scope of the “composition” described above.

The term “and/or” as used herein includes the meanings of “and”, “or”, and “all or any other combination of elements linked by the term”.

The term “BCMA” herein refers to the B cell maturation antigen, which is a member of the tumor necrosis factor receptor family. BCMA is mainly expressed on the surface of late-stage B cells, short-lived proliferating plasmablasts, and long-lived plasma cells, while it is not expressed in naive B cells, CD34-positive hematopoietic stem cells, and other normal tissue cells. However, it is highly expressed in MM cells, and plays a critical role in the survival, proliferation, metastasis, and drug resistance of MM cells by mediating downstream signaling pathways. BCMA is therefore an ideal antigen target for treating MM. An exemplary human BCMA sequence can be found in GenBank Protein Accession No: NP_001183.2.

The term GPRC5D herein refers to the G-protein coupled receptor family C group 5 member D, an orphan receptor, which is a 7-transmembrane protein and currently has no known ligand. The GPRC5D is highly expressed on the surface of primary multiple myeloma cells, while its expression in normal tissues is limited to the hair follicle region. Studies have shown that 65% of patients with multiple myeloma exceed the GPRC5D expression threshold of 50%, and by virtue of this characteristic, GPRC5D is a potential target for treating MM. An exemplary human GPRC5D sequence can be found in GenBank Protein Accession No: NP_061124.1.

As used herein, the term “chimeric antigen receptor (CAR)” refers to an artificial immune effector cell surface receptor that is engineered and expressed on an immune effector cell and specifically binds to an antigen, comprising, at least: (1) an extracellular antigen-binding domain, e.g., a variable heavy or light chain of an antibody, (2) a transmembrane domain that anchors the CAR on the immune effector cell, and (3) an intracellular signaling domain. The CAR is capable of redirecting T cells and other immune effector cells to a selected target, e.g., a cancer cell, in a non-MHC-restricted manner using the extracellular antigen-binding domain.

The term “signal peptide” herein refers to a fragment of a protein or polypeptide that is used to direct the protein or polypeptide into the secretory pathway, and transfer same to the cell membrane and/or cell surface. In some embodiments, the signal peptide is a CD8a signal peptide, and optionally, the CD8a signal peptide has at least 80% (e.g., at least 85%, at least 90%, at least 91%, at least 92%, at least 93%, at least 94%, at least 95%, at least 96%, at least 97%, at least 98%, at least 99%, or 100%) identity to the following sequence: MALPVTALLLPLALLLHAARP.

As used herein, the “transmembrane (TM) region” of a chimeric antigen receptor refers to a polypeptide structure that enables the chimeric antigen receptor to be expressed on the surface of an immune cell (e.g., a lymphocyte, an NK cell, or an NKT cell) and directs the cellular response of the immune cell against a target cell. The transmembrane domain may be natural or synthetic, and may be derived from any membrane-bound protein or transmembrane protein. The transmembrane domain can transduce a signal when the chimeric antigen receptor binds to a target antigen.

As used herein, the “hinge region” of a chimeric antigen receptor generally refers to any oligopeptide or polypeptide that serves to connect a transmembrane region and an antigen-binding region. In particular, the hinge region serves to provide greater flexibility and accessibility to the antigen-binding region. The hinge region may be derived in whole or in part from a natural molecule, e.g., an extracellular domain derived in whole or in part from CD8, CD4, or CD28, or derived in whole or in part from an antibody constant region. Alternatively, the hinge region may be either a synthetic sequence corresponding to a naturally occurring hinge sequence, or a completely synthetic hinge sequence.

As used herein, the term “intracellular signaling domain” refers to a portion of a protein that transduces an effector function signal and directs a cell to exert a specified function. The intracellular signaling domain is responsible for the intracellular primary signaling after the antigen-binding domain binds to an antigen, resulting in the activation of the immune cell and immune response. In other words, the intracellular signaling domain is responsible for activating at least one of the normal effector functions of the immune cell in which the CAR is expressed. Exemplary intracellular signaling domains include CD32.

As used herein, the term “co-stimulatory domain” refers to the intracellular signaling domain of a co-stimulatory molecule. The co-stimulatory molecules are cell surface molecules other than antigen receptors or Fc receptors, and the cell surface molecules provide the second signal required for the effective activation and functionality of T lymphocytes upon binding to an antigen.

The term “immune effector cell” or “effector cell” as used herein refers to a cell involved in an immune response, e.g., promoting an immune effector response. Examples of the immune effector cells include T cells, e.g., α/β T cells and γ/δ T cells, B cells, natural killer (NK) cells, natural killer T (NKT) cells, mast cells, and myeloid-derived phagocytes.

The terms “T lymphocyte” and “T cell” herein are used interchangeably, and refer to the major type of white blood cells, which mature in the thymus and have multiple functions in the immune system, including recognizing specific foreign antigens in vivo, and activating and inactivating other immune cells in an MHC class I restricted manner. The T cell may be any T cell, for example, a cultured T cell such as a primary T cell, or a T cell from a cultured T cell line such as Jurkat or SupT1, or a T cell obtained from a mammal. The T cell may be a CD3+ cell. The T cell may be any type of T cell, and may be at any developmental stage, including but not limited to a CD4+/CD8+ double positive T cell, a CD4+ helper T cell (e.g., Th1 and Th2 cells), a CD8+ T cell (e.g., a cytotoxic T cell), a peripheral blood mononuclear cell (PBMC), a peripheral blood leukocyte (PBL), a tumor infiltrating lymphocyte (TIL), a memory T cell, a naive T cell, a regulatory T cell, a gamma delta T cells/γδ T cell, and the like. Other types of helper T cells include, for example, Th3 (Treg), Th17, Th9, or Tfh cells. Other types of memory T cells include, for example, central memory T cells (Tcm cells), and effector memory T cells (Tem cells and TEMRA cells). The T cells may also refer to genetically engineered T cells, e.g., T cells modified to express a T cell receptor (TCR) or a chimeric antigen receptor (CAR). The T cells or T cell-like effector cells may also be differentiated from stem cells or progenitor cells. T cell-like derived effector cells may in some aspects have a T cell lineage, while also having one or more functional characteristics not present in primary T cells. The term “NK cell” or “natural killer cell” herein refers to a subpopulation of peripheral blood lymphocytes defined by the expression of CD56 or CD16 and the absence of T cell receptor (CD3). As used herein, the terms “adaptive NK cells” and “memory NK cells” are interchangeable, and refer to a subpopulation of the NK cells with a phenotype of CD3⁻ and CD56+ and the expression of at least one of NKG2C, CD57, and optionally CD16, but the absence of the expression of one or more of: PLZF, SYK, FceRγ, and EAT-2. In some embodiments, the isolated CD56⁺ NK cell subpopulation comprises the expression of CD16, NKG2C, CD57, NKG2D, NCR ligands, NKp30, NKp40, NKp46, activating and inhibitory KIRs, NKG2A, and/or DNAM-1. CD56⁺ may indicate a low or high expression. The NK cells or NK cell-like effector cells may be differentiated from stem cells or progenitor cells. NK cell-like derived effector cells may in some aspects have an NK cell lineage, while also having one or more functional characteristics not present in primary T cells. As used herein, the term “NKT cell” or “natural killer T cell” refers to a T cell limited by CD1d, which expresses a T cell receptor (TCR). Unlike conventional T cells that detect peptide antigens presented by conventional major histocompatibility (MHC) molecules, NKT cells recognize lipid antigens presented by CD1d (a non-classical MHC molecule). Two types of NKT cells are recognized. Constant or type I NKT cells express a very limited TCR library: the binding of a classical a chain (Vα24-Jα18 in humans) to a β chain with a limited spectrum (Vβ11 in humans). The second NKT cell population (known as non-classical or non-constant type II NKT cells) shows a more heterogeneous TCRαβ utilization rate. Type I NKT cells are considered suitable for immunotherapy. Adaptive or constant (type I) NKT cells can be identified by the expression of at least one or more of the following markers: TCR Va24-Ja18, Vb11, CD1d, CD3, CD4, CD8, aGalCer, CD161, and CD56.

The term “autologous” herein refers to any material that is derived from an individual and is subsequently reintroduced into the same individual. The term “allogenic” refers to grafts derived from different individuals of the same species.

The term “specifically bind to” herein refers to that an antigen-binding molecule (e.g., an antibody) specifically binds to an antigen and substantially identical antigens, generally with high affinity, but does not bind to unrelated antigens with high affinity. The affinity is generally reflected in an equilibrium dissociation constant (KD), where a relatively low KD indicates a relatively high affinity. In the case of antibodies, a high affinity generally refers to a KD of about 10⁻⁶ M or less, about 10⁻⁷ M or less, about 10⁻⁸ M or less, about 1×10⁻⁹ M or less, 1×10⁻¹⁰ M or less, 1×10⁻¹¹ M or less, or 1×10⁻¹² M or less. The KD is calculated as follows: KD=Kd/Ka, where Kd represents the dissociation rate and Ka represents the association rate. The equilibrium dissociation constant KD can be measured by methods well known in the art, such as surface plasmon resonance (e.g., Biacore) or equilibrium dialysis. Illustratively, KD can be obtained by the method as described in the examples herein.

The term “antibody” herein is used in its broadest sense and refers to a polypeptide or a combination of polypeptides that comprises sufficient sequence from an immunoglobulin heavy chain variable region and/or sufficient sequence from an immunoglobulin light chain variable region to be capable of specifically binding to an antigen. The “antibody” herein encompasses various forms and various structures as long as they exhibit the desired antigen-binding activity. The “antibody” herein includes alternative protein scaffolds or artificial scaffolds having grafted complementarity determining regions (CDRs) or CDR derivatives. Such scaffolds include antibody-derived scaffolds comprising mutations introduced to, for example, stabilize the three-dimensional structure of the antibody, and fully synthetic scaffolds comprising, for example, biocompatible polymers. See, e.g., Korndorfer et al., 2003, Proteins: Structure, Function, and Bioinformatics, 53 (1): 121-129 (2003); and Roque et al., Biotechnol. Prog. 20:639-654 (2004). Such scaffolds may also include non-antibody derived scaffolds, such as scaffold proteins known in the art to be useful for grafting CDRs, including, but not limited to, tenascin, fibronectin, peptide aptamers, and the like.

The term “antibody” herein includes a typical “four-chain antibody”, which is an immunoglobulin consisting of two heavy chains (HCs) and two light chains (LCs). The heavy chain refers to a polypeptide chain consisting of, from the N-terminus to the C-terminus, a heavy chain variable region (VH), a heavy chain constant region CH1 domain, a hinge region (HR), a heavy chain constant region CH2 domain, a heavy chain constant region CH3 domain; moreover, when the full-length antibody is of IgE isoform, the heavy chain optionally further comprises a heavy chain constant region CH4 domain. The light chain is a polypeptide chain consisting of, from the N-terminus to the C-terminus, a light chain variable region (VL) and a light chain constant region (CL). The heavy chains are connected to each other and to the light chains through disulfide bonds to form a Y-shaped structure. The heavy chain constant regions of immunoglobulins differ in their amino acid composition and arrangement, and thus in their antigenicity. Accordingly, the “immunoglobulin” herein can be divided into five classes, or isoforms of immunoglobulins, i.e., IgM, IgD, IgG, IgA, and IgE, with their corresponding heavy chains being μ, δ, γ, α, and ε chains, respectively. The Ig of the same class may also be divided into different subclasses according to the differences in the amino acid composition of the hinge regions and the number and locations of disulfide bonds in the heavy chains. For example, IgG may be divided into IgG1, IgG2, IgG3, and IgG4, and IgA may be divided into IgA1 and IgA2. The light chains can be divided into κ or λ chains according to the differences in the constant regions. Each of the five classes of Ig may have a κ chain or a λ chain.

The “antibody” herein also includes antibodies that do not comprise a light chain, e.g., heavy chain antibodies (HCAbs) produced by Camelus dromedarius, Camelus bactrianus, Lama glama, Lama guanicoe, Vicugna pacos, and the like, as well as immunoglobulin new antigen receptors (IgNARs) found in Chondrichthyes species, e.g., shark.

The “antibody” herein may be derived from any animal, including, but not limited to, human and non-human animals which may be selected from primates, mammals, rodents, and vertebrates, such as Camelidae species, Lama glama, Lama guanicoe, Vicugna pacos, sheep, rabbits, mice, rats, or Chondrichthyes species (e.g., shark).

The terms “antigen-binding fragment” and “antibody fragment” herein are used interchangeably and refer to a fragment that does not have the entire structure of an intact antibody, but comprises only a portion of the intact antibody or a variant of the portion that retains the ability to bind to an antigen. The “antigen-binding fragment” or “antibody fragment” herein includes, but is not limited to, a Fab, a Fab′, a Fab′-SH, a F(ab′)₂, an scFv, and a VHH.

An intact antibody can be digested by papain to produce two identical antigen-binding fragments, known as “Fab” fragments, each of which contains a heavy chain variable domain and a light chain variable domain, as well as a light chain constant domain and a first heavy chain constant domain (CH1). Thus, the term “Fab fragment” herein refers to an antibody fragment comprising a light chain fragment comprising the VL domain and the constant domain (CL) of a light chain, and the VH domain and the first constant domain (CH1) of a heavy chain. The Fab′ fragment differs from the Fab fragment by the addition of a few residues (including one or more cysteines from an antibody hinge region) at the carboxyl terminus of the heavy chain CH1 domain. The Fab′-SH is a Fab′ fragment in which the cysteine residue in the constant domain carries a free thiol group. The pepsin treatment produces a F(ab′)₂ fragment having two antigen-binding sites (two Fab fragments) and a portion of the Fc region.

The term “scFv” (single-chain variable fragment) herein refers to a single polypeptide chain comprising VL and VH domains, wherein the VL and VH are linked through a linker (see, e.g., Bird et al., Science 242:423-426 (1988); Huston et al., Proc. Natl. Acad. Sci. USA 85:5879-5883 (1988); and Pluckthun, The Pharmacology of Monoclonal Antibodies, Vol. 113, Roseburg and Moore Ed., Springer-Verlag, New York, pp 269-315 (1994)). Such scFv molecules may have a general structure: NH₂-VL-linker-VH-COOH or NH₂-VH-linker-VL-COOH. An appropriate linker in the prior art consists of GGGGS amino acid sequence repeats or a variant thereof. For example, a linker having the amino acid sequence (GGGGS) 4 can be used, and variants thereof can also be used (Holliger et al. (1993), Proc. Natl. Acad. Sci. USA 90:6444-6448). Other linkers that can be used in the present disclosure are described in Alfthan et al. (1995), Protein Eng. 8:725-731; Choi et al. (2001), Eur. J. Immunol. 31:94-106; Hu et al. (1996), Cancer Res. 56:3055-3061; Kipriyanov et al. (1999), J. Mol. Biol. 293:41-56; and Roovers et al. (2001), Cancer Immunol. In some cases, there may also be disulfide bonds between the VH and VL of the scFv, forming a disulfide-linked Fv (dsFv).

The term “nanobody” herein refers to a heavy chain antibody naturally lacking a light chain present in a camel or the like, and the cloning of its variable region can give a single domain antibody only consisting of a heavy chain variable region (also called VHH (variable domain of heavy chain of heavy chain antibody)), which is the smallest functional antigen-binding fragment.

The terms “VHH domain” and “single domain antibody” (sdAb) herein have the same meaning and are used interchangeably, and refer to a single domain antibody consisting of only one heavy chain variable region constructed by cloning a variable region of a heavy chain antibody, which is the smallest antigen-binding fragment having the complete functionality. Generally, a single domain antibody consisting of only one heavy chain variable region is constructed by acquiring a heavy chain antibody naturally lacking a light chain and a heavy chain constant region 1 (CH1) and then cloning the variable region of the antibody heavy chain.

The term “variable region” herein refers to a region of a heavy or light chain of an antibody involved in the binding of the antibody to an antigen. The “heavy chain variable region” is used interchangeably with “VH” and “HCVR”, and the “light chain variable region” is used interchangeably with “VL” and “LCVR”. Heavy and light chain variable domains (VH and VL, respectively) of natural antibodies generally have similar structures, each of which contains four conservative framework regions (FRs) and three hypervariable regions (HVRs). See, e.g., Kindt et al., Kuby Immunology, 6th ed., W. H. Freeman and Co., p. 91 (2007). A single VH or VL domain may be sufficient to provide antigen-binding specificity. The terms “complementarity determining region” and “CDR” herein are used interchangeably and generally refer to a hypervariable region (HVR) of a heavy chain variable region (VH) or a light chain variable region (VL), which is also known as the complementarity determining region as it is precisely complementary to an epitope in spatial structures, wherein the heavy chain variable region CDR may be abbreviated as HCDR and the light chain variable region CDR may be abbreviated as LCDR. The terms “framework region” or “FR” are used interchangeably and refer to those amino acid residues of an antibody heavy chain variable region or light chain variable region other than CDRs. Generally, a typical antibody variable region consists of 4 FRs and 3 CDRs in the following order: FR1-CDR1-FR2-CDR2-FR3-CDR3-FR4.

For further description of the CDRs, see Kabat et al., J. Biol. Chem., 252:6609-6616 (1977); Kabat et al., United States Department of Health and Human Services, Sequences of proteins of immunological interest (1991); Chothia et al., J. Mol. Biol. 196:901-917 (1987); Al-Lazikani B. et al., J. Mol. Biol., 273:927-948 (1997); MacCallum et al., J. Mol. Biol. 262:732-745 (1996); Abhinandan and Martin, Mol. Immunol., 45:3832-3839 (2008); Lefranc M. P. et al., Dev. Comp. Immunol., 27:55-77 (2003); and Honegger and Plückthun, J. Mol. Biol., 309:657-670 (2001). The “CDR” herein may be labeled and defined in a manner well known in the art, including, but not limited to, Kabat numbering scheme, Chothia numbering scheme, or IMGT numbering scheme; the tool websites used include, but are not limited to, AbRSA site (http://cao.labshare.cn/AbRSA/cdrs.php), abYsis site (www.abysis.org/abysis/sequence_input/key_annotation/key_annotation.cgi), and IMGT site (http://www.imgt.org/3Dstructure-DB/cgi/DomainGapAlign.cgi #results). The CDR herein includes overlaps and subsets of amino acid residues defined in different ways.

The term “Kabat numbering scheme” herein generally refers to the immunoglobulin alignment and numbering scheme proposed by Elvin A. Kabat (see, e.g., Kabat et al., Sequences of Proteins of Immunological Interest, 5th Ed. Public Health Service, National Institutes of Health, Bethesda, Md., 1991).

The term “Chothia numbering scheme” herein generally refers to the immunoglobulin numbering scheme proposed by Chothia et al., which is a classical rule for identifying CDR region boundaries based on the position of structural loop regions (see, e.g., Chothia & Lesk (1987) J. Mol. Biol. 196:901-917; Chothia et al., (1989) Nature 342:878-883).

The term “IMGT numbering scheme” herein generally refers to a numbering scheme based on the international ImMunoGeneTics information system (IMGT) initiated by Lefranc et al., See Lefranc et al., Dev. Comparat. Immunol. 27:55-77, 2003.

As used herein, the terms “percent (%) sequence consistency” and “percent (%) sequence identity” are interchangeable, and refer to the percentage of identity between amino acid (or nucleotide) residues of a candidate sequence and amino acid (or nucleotide) residues of a reference sequence after aligning the sequences and introducing gaps, if necessary, to achieve maximum percent sequence identity (e.g., gaps may be introduced in one or both of the candidate sequence and the reference sequence for optimal alignment, and non-homologous sequences may be omitted for the purpose of comparison). Alignment may be carried out in a variety of ways well known to those skilled in the art for the purpose of determining percent sequence identity, for example, using publicly available computer software such as BLAST, ALIGN, or Megalign (DNASTAIi) software. Those skilled in the art can determine appropriate parameters for measuring alignment, including any algorithms required to achieve maximum alignment of the full length of the aligned sequences. For example, a reference sequence aligned for comparison with a candidate sequence may show that the candidate sequence exhibits 50% to 100% sequence identity over the full length of the candidate sequence or a selected portion of contiguous amino acid (or nucleotide) residues of the candidate sequence. The length of the candidate sequence aligned for the purpose of comparison may be, e.g., at least 30% (e.g., 30%, 40%, 50%, 60%, 70%, 80%, 90%, or 100%) of the length of the reference sequence. When a position in the candidate sequence is occupied by the same amino acid (or nucleotide) residue at the corresponding position in the reference sequence, then the molecules are identical at that position.

As used herein, the term “at least 80% identity” is preferably 85%, 90%, 91%, 92%, 93%, 94%, 95%, 96%, 97%, 98%, 99%, or 100% identity.

As used herein, the term “mutation” includes insertion mutation, deletion mutation, and substitution mutation, and preferably, the substitution mutation is a conservative amino acid substitution. As used herein, the term “conservative amino acid” generally refers to amino acids that belong to the same class or have similar characteristics (e.g., charge, side chain size, hydrophobicity, hydrophilicity, backbone conformation, and rigidity). Illustratively, the amino acids in each of the following groups belong to conservative amino acid residues of each other, and substitutions of amino acid residues within the groups belong to conservative amino acid substitutions:

Illustratively, the following six groups are examples of amino acids that are considered to be conservative replacements of each other:
1) alanine (A), serine(S), and threonine (T);
2) aspartic acid (D) and glutamic acid (E);
3) asparagine (N) and glutamine (Q);
4) arginine (R), lysine (K), and histidine (H);
5) isoleucine (I), leucine (L), methionine (M), and valine (V); and
6) phenylalanine (F), tyrosine (Y), and tryptophan (W).

As used herein, the term “at most 5 mutations” is preferably at most 4, 3, 2, 1, or 0 mutations.

As used herein, the term “at most 9 mutations” is preferably at most 8, 7, 6, 5, 4, 3, 2, 1, or 0 mutations.

As used herein, the term “at most 10 mutations” is preferably at most 10, 9, 8, 7, 6, 5, 4, 3, 2, 1, or 0 mutations.

As used herein, the term “at most 25 mutations” is preferably at most 24, 23, 22, 21, 20, 19, 18, 17, 16, 15, 14, 13, 12, 11, 10, 9, 8, 7, 6, 5, 4, 3, 2, 1, or 0 mutations.

As used herein, the term “at most 30 mutations” is preferably at most 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 mutations.

As used herein, the term “at most 50 mutations” is preferably at most 49, 48, 47, 46, 45, 44, 43, 42, 41, 40, 39, 38, 37, 36, 35, 34, 33, 32, 31, 20, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 mutations.

As used herein, the term “at most 100 mutations” is preferably at most 99, 98, 97, 96, 95, 94, 93, 92, 91, 90, 89, 88, 87, 86, 85, 84, 83, 82, 81, 70, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 20, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 mutations.

As used herein, the term “at most 150 mutations” is preferably at most 149, 148, 147, 146, 145, 144, 143, 142, 141, 140, 139, 138, 137, 136, 135, 134, 133, 132, 131, 120, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 70, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 20, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 mutations.

As used herein, the term “vector” is a composition of matter that comprises an isolated nucleic acid and can be used to deliver the isolated nucleic acid to the interior of a cell. A variety of vectors are known in the art, including but not limited to, linear polynucleotides, polynucleotides associated with ionic or amphiphilic compounds, plasmids, and viruses. Thus, the term “vector” includes an autonomously replicating plasmid or virus. The term should also be construed to include non-plasmid and non-viral compounds that facilitate the transfer of nucleic acids into cells, e.g., polylysine compounds and liposomes. Examples of viral vectors include, but are not limited to, adenoviral vectors, adeno-associated viral vectors, retroviral vectors, and the like.

As used herein, the terms “subject” and “patient” refer to an organism that receives a treatment for a particular disease or disorder (e.g., a cancer or an infectious disease) as described herein. Examples of the subject and the patient include mammals, such as human, primate, pig, goat, rabbit, hamster, cat, dog, guinea pig, members of the bovine family (such as cattle, bison, buffalo, elk, yak, etc.), cattle, sheep, horse, bison, etc., that receive a treatment for a disease or disorder (e.g., a cell proliferative disorder, such as a cancer or an infectious disease).

As used herein, the term “treatment” refers to surgical or therapeutic treatment for the purpose of preventing or slowing (reducing) the progression of an undesirable physiological or pathological change, such as a cell proliferative disorder (e.g., a cancer or an infectious disease), in a subject being treated. Beneficial or desired clinical results include, but are not limited to, alleviation of symptoms, decrease in severity of disease, stabilization (i.e., not worsening) of state of disease, delay or slowing of disease progression, amelioration or palliation of state of disease, and remission (whether partial or total), whether detectable or undetectable. Subjects in need of a treatment include those already with a disorder or disease, as well as those who are susceptible to a disorder or disease or those who intend to prevent a disorder or disease. When referring to terms such as slowing, alleviation, decrease, palliation, and remission, their meanings also include elimination, disappearance, nonoccurrence, etc.

As used herein, the term “effective amount” refers to an amount of a therapeutic agent that is effective in preventing or alleviating the symptoms of a disease or the progression of the disease when administered to a cell, tissue, or subject alone or in combination with another therapeutic agent. The “effective amount” also refers to an amount of a compound that is sufficient to alleviate the symptoms, e.g., to treat, cure, prevent, or alleviate the related medical disorders, or to increase the rates at which such disorders are treated, cured, prevented, or alleviated. When the active ingredient is administered alone to an individual, the therapeutically effective dose refers to the amount of the ingredient alone. When a combination is used, the therapeutically effective dose refers to the combined amounts of the active ingredients that produce the therapeutic effect, whether administered in combination, sequentially, or simultaneously.

### BRIEF DESCRIPTION OF THE DRAWINGS

FIG. 1 shows a schematic structural diagram of CAR-NK comprising different NKG2D TM region elements.

FIG. 2 shows the CAR expression rates in BCMA-CAR NK cells.

FIG. 3 shows the CAR expression rates in ROR1-CAR NK cells.

FIG. 4 shows the proliferation folds of BCMA-CAR NK cells.

FIG. 5 shows the proliferation folds of ROR1 CAR NK cells.

FIG. 6 shows the flow cytometry results of MOLP8 and NCI H929-hBCMA-KO.

FIG. 7 shows the killing effects of BCMA-CAR NK on MOLP8 cells after a multiple-run killing assay.

FIGS. 8A-8C show the killing effects of ROR1 CAR NK on A549, RPMI 8226, and 7860 after a multiple-run killing assay.

FIG. 9 shows a schematic structural diagram of BCMA-CAR-NK-6 (BCMA-CAR6).

FIG. 10 shows the CAR expression rates in BCMA-CAR NK cells in animal studies.

FIGS. 11A-11E show the anti-tumor efficacy study of BCMA-CAR-NK in different forms in an NCI H929-luc multiple myeloma animal model. FIG. 11A shows the results of bioluminescence signal detection in animal tumors; FIG. 11B shows the weight changes (%) in animals; FIG. 11C shows the amount of bio-signal photons in animal tumors; FIG. 11D shows the tumor growth inhibition (%) in animals; FIG. 11E shows the survival rates (%) of animals.

FIG. 12 shows schematic structural diagrams of dual-target chimeric antigen receptors (BI-CARs) targeting BCMA and GPRC5D.

FIG. 13 shows a schematic structural diagram of a dual-target chimeric antigen receptor (BI-CAR) targeting BCMA and GPRC5D in the form of NKG2D TM.

FIG. 14 shows the summary of BI-CAR-NK cell proliferation folds.

FIGS. 15A and 15B show the results of the multiple-run killing assay of BI-CAR-NK against MOLP8 cells and RPMI 8226 cells.

FIG. 16 shows schematic structural diagrams of dual-target chimeric antigen receptors (BI-CARs) targeting BCMA and GPRC5D.

FIG. 17 shows the summary of BI-CAR-NK cell proliferation folds.

FIGS. 18A-18F show the results of the multiple-run killing assay of BI-CAR-NK against NCI H929 cells, RPMI 8226 cells, MOLP8 cells, and heterogeneous tumor cells.

### DETAILED DESCRIPTION

The present disclosure is further described below with reference to specific examples; the advantages and features of the present disclosure will become more apparent with the description. Experimental procedures without specified conditions in the examples are conducted according to conventional conditions or conditions recommended by the manufacturers. Reagents or instruments without specified manufacturers used herein are conventional products that are commercially available.

The examples herein are illustrative only, and do not limit the scope of the present disclosure in any way. It will be appreciated by those skilled in the art that various modifications or substitutions may be made to the technical solutions of the present disclosure in form and details without departing from the spirit and scope of the present disclosure, and that these modifications and substitutions shall fall within the protection scope of the present disclosure.

Unless otherwise stated, the target cells described in the following examples were transfected with the luciferase gene to express luciferase. The fluorescence intensity was detected by using a luciferase reporter gene assay reagent to represent the cell viability and the killing effect of NK cells. The killing rate calculation formula is as follows:

Killing rate=(target cell well reading−assay well reading)/target cell well reading×100%.

Unless otherwise specified, the target cells used in the following examples are A549 cells, 786-O cells, RPMI 8226 cells, MOLP8 cells, and H929-hBCMA-KO cells, all of which were transfected with the luciferase reporter gene by conventional gene manipulation methods, wherein the H929-hBCMA-KO cells were H929 cells with BCMA gene knockout using conventional gene manipulation methods.

### Example 1. Screening and Preparation of Antibodies

### 1. Screening and Identification of BCMA Antibodies

Vicugna pacos was immunized with a protein containing the extracellular domain of the human BCMA as antigens. The peripheral blood after the fourth and fifth immunizations (antibody titer and specificity had been verified by ELISA) was taken, and the PBMCs were isolated. The total RNA was extracted, and the VHH gene fragments were amplified by reverse transcription and nested PCR. A phage library was constructed, and clones that were cross-positive with human and monkey BCMAs were panned and identified. The variable region sequences of the positive clones were obtained by sequencing, and designated as VHH1 and VHH2, the CDR region sequences of which were determined by the Kabat numbering scheme and the Chothia numbering scheme (http://www.abysis.org/abysis/sequence input/key annotation/key annotation.cgi) and the IMGT numbering scheme (https://www.imgt.org/3Dstructure-DB/cgi/DomainGapAlign.cgi).

By alignment with the IMGT database (http://imgt.cines.fr) for germline genes from heavy and light chain variable regions of human antibodies, germline genes (IGHV3-64*04 and IGHJ3*01) from heavy chain variable region, with high homology to the VHH1 antibody, or germline genes (IGHV3-7*01 and IGHJ6*01) from heavy chain variable region, with high homology to the VHH2 antibody, were selected as templates, and the CDRs (as determined by the IMGT numbering scheme) of the VHH1 or VHH2 were separately grafted into corresponding human templates to give variable region sequences in the order of FR1-CDR1-FR2-CDR2-FR3-CDR3-FR4. Key amino acids in a framework sequence were back-mutated to amino acids corresponding to the VHH nanobody as needed to ensure the original affinity. If the antibody had sites susceptible to chemical modification, point mutations were performed at these sites to eliminate modification risks. Humanized antibodies hu-VHH1 and hu-VHH2 were obtained. The sequences and CDRs of the VHH antibodies and the humanized antibodies thereof are shown in Tables 1 and 2.

The VHH1, VHH2, hu-VHH1, and hu-VHH2 sequences were recombined into an expression vector for human IgG1 Fc, and chimeric antibodies and humanized antibodies VHH1-hFc, VHH2-hFc, hu-VHH1-hFc, and hu-VHH2-hFc were obtained after expression and purification. As verified by ELISA and FACS, the antibodies described above showed good binding activities to the human BCMA protein and the monkey BCMA protein, and showed good bindings to H929 and U266 cells which endogenously express BCMA. The affinity detection results further verified that VHH1-hFc, VHH2-hFc, hu-VHH1-hFc, and hu-VHH2-hFc showed high affinity to the human BCMA protein, with KD values of 5.27E-10 M (VHH1-hFc), 7.16E-11 M (VHH2-hFc), 6.13E-10 M (hu-VHH1-hFc), and 8.82E-11 M (hu-VHH2-hFc), respectively.

TABLE 1
Sequences of antibodies

| | SEQ | |
|---|---|---|
| Antibody | ID NO: | Sequence |
| VHH1 | 1 | QVQLVESGGGLVQPGGSLRLSCSGSGFTLDYYMIGWFRQAPGKEREGLSSISPAD |
| | | GSTYYADSVKGRFTISRDSAKNTVYLQMNNLKPEDTALYYCAAGNEATISWGFGP |
| | | WEYDYWGQGTQVTVSS |
| VHH2 | 2 | QVQLVESGGGLVQSRGSLRLSCAASESISSIHIMAWYRRAPGKQRELVAGIRNDGS |
| | | TVYADSAKGRFTISRDNAKNTVYLQMNSLKPEDTAVYYCNADQGFGSYSEWERR |
| | | SRWGQGTEVTVSS |
| hu-VHH1 | 3 | EVQLVESGGGLVQPGGSLRLSCSASGFTLDYYMIGWFRQAPGKEREGLSSISPADG |
| | | STYYADSVKGRFTISRDSSKNTVYLQMNSLRAEDTAVYYCAAGNEATISWGFGPW |
| | | EYDYWGQGTMVTVSS |
| hu-VHH2 | 4 | EVQLVESGGGLVQPGGSLRLSCAASESISSIHIMAWYRQAPGKQRELVAGIRNDGS |
| | | TVYVDSVKGRFTISRDNAKNSVYLQMNSLRAEDTAVYYCNADQGFGSYSEWERR |
| | | SRWGQGTTVTVSS |
| VHH1-hFc | 5 | QVQLVESGGGLVQPGGSLRLSCSGSGFTLDYYMIGWFRQAPGKEREGLSSISPAD |
| | | GSTYYADSVKGRFTISRDSAKNTVYLQMNNLKPEDTALYYCAAGNEATISWGFGP |
| | | WEYDYWGQGTQVTVSSEPKSSDKTHTCPPCPAPELLGGPSVFLFPPKPKDTLMISR |
| | | TPEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSREEMTKNQV |
| | | SLTCLVKGFYPSDIAVEWESNGQPENNYKTTPPVLDSDGSFFLYSKLTVDKSRWQQ |
| | | GNVFSCSVMHEALHNHYTQKSLSLSPGK |
| VHH2-hFc | 6 | QVQLVESGGGLVQSRGSLRLSCAASESISSIHIMAWYRRAPGKQRELVAGIRNDGS |
| | | TVYADSAKGRFTISRDNAKNTVYLQMNSLKPEDTAVYYCNADQGFGSYSEWERR |
| | | SRWGQGTEVTVSSEPKSSDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQD |
| | | WLNGKEYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRWQQGNV |
| | | FSCSVMHEALHNHYTQKSLSLSPGK |
| hu-VHH1-hFc | 7 | EVQLVESGGGLVQPGGSLRLSCSASGFTLDYYMIGWFRQAPGKEREGLSSISPADG |
| | | STYYADSVKGRFTISRDSSKNTVYLQMNSLRAEDTAVYYCAAGNEATISWGFGPW |
| | | EYDYWGQGTMVTVSSEPKSSDKTHTCPPCPAPELLGGPSVFLFPPKPKDTLMISRT |
| | | PEVTCVVVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSREEMTKNQVS |
| | | LTCLVKGFYPSDIAVEWESNGQPENNYKTTPPVLDSDGSFFLYSKLTVDKSRWQQ |
| | | GNVFSCSVMHEALHNHYTQKSLSLSPGK |
| hu-VHH2-hFc | 8 | EVQLVESGGGLVQPGGSLRLSCAASESISSIHIMAWYRQAPGKQRELVAGIRNDGS |
| | | TVYVDSVKGRFTISRDNAKNSVYLQMNSLRAEDTAVYYCNADQGFGSYSEWERR |
| | | SRWGQGTTVTVSSEPKSSDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQD |
| | | WLNGKEYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRWQQGNV |
| | | FSCSVMHEALHNHYTQKSLSLSPGK |

TABLE 2
CDRs of VHH1 and VHH2

| | Numbering | | | |
|---|---|---|---|---|
| Antibody | scheme | CDR1 | CDR2 | CDR3 |
| VHH1 or | Kabat | YYMIG | SISPADGSTYYADSVKG | GNEATISWGFGPWEYDY |
| hu-VHH1 | | (SEQ ID NO: 9) | (SEQ ID NO: 10) | (SEQ ID NO: 11) |
| | Chothia | GFTLDYY | SPADGS | GNEATISWGFGPWEYDY |
| | | (SEQ ID NO: 12) | (SEQ ID NO: 13) | (SEQ ID NO: 11) |
| | IMGT | GFTLDYYM | ISPADGST | AAGNEATISWGFGPWEYDY |
| | | (SEQ ID NO: 14) | (SEQ ID NO: 15) | (SEQ ID NO: 16) |
| VHH2 | Kabat | IHIMA | GIRNDGSTVYADSAKG | DQGFGSYSEWERRSR |
| | | (SEQ ID NO: 17) | (SEQ ID NO: 18) | (SEQ ID NO: 19) |
| | Chothia | ESISSIH | RNDGS | DQGFGSYSEWERRSR |
| | | (SEQ ID NO: 20) | (SEQ ID NO: 21) | (SEQ ID NO: 19) |
| | IMGT | ESISSIHI | IRNDGST | NADQGFGSYSEWERRSR |
| | | (SEQ ID NO: 22) | (SEQ ID NO: 23) | (SEQ ID NO: 24) |
| hu-VHH2 | Kabat | IHIMA | GIRNDGSTVYVDSVKG | DQGFGSYSEWERRSR |
| | | (SEQ ID NO: 17) | (SEQ ID NO: 25) | (SEQ ID NO: 19) |
| | Chothia | ESISSIH | RNDGS | DQGFGSYSEWERRSR |
| | | (SEQ ID NO: 20) | (SEQ ID NO: 21) | (SEQ ID NO: 19) |
| | IMGT | ESISSIHI | IRNDGST | NADQGFGSYSEWERRSR |
| | | (SEQ ID NO: 22) | (SEQ ID NO: 23) | (SEQ ID NO: 24) |

### 2. Screening and Identification of ROR1 Antibodies

Mice were immunized with a human ROR-1 fusion protein (ACRO, Cat. RO1-H5250), and a positive clone ROR1-1 was screened by splenocyte fusion and hybridoma techniques. The variable region sequences of the antibody were obtained by sequencing. The CDR region sequences of the antibody were determined by the Kabat, Chothia, and IMGT numbering schemes. The nucleic acid sequence encoding ROR1-1-scFv was cloned into an expression vector containing a signal peptide and human Fc, and a chimeric antibody ROR1-1-hFc was obtained through expression and purification. As identified by ELISA and FACS, the chimeric antibody specifically bound to the human ROR1 protein and MDA-MB-231 cells endogenously expressing ROR1. The affinity detection results further verified that the chimeric antibody bound to the human ROR1 protein with a KD value of 3.23E-07 M. The specific sequences are detailed in Tables 3 and 4.

TABLE 3
Sequences of variable regions of ROR1 antibody

| | SEQ ID | |
|---|---|---|
| Antibody | NO: | Sequence |
| ROR1-1-VH | 26 | DLQLQESGPGLVKPSQSLSLTCSVTGYSITSGYDWNWIRQFPGNKLEWMGYINY |
| | | DGSNNYNPSLKNRISITRDTSKNQFFLRLSSVTTEDTATYFCARGILRGYFDYWG |
| | | QGTTLTVSS |
| ROR1-1-VL | 27 | EIVLTQSPGSLAVSLGQRATISCRASESVDNFGLSFMHWYQQKPGQPPKLLIYRAS |
| | | NLESGIPARFSGSGSRTDFTLTINPVETDDVATYYCQQSNKDPYTFGGGTKLEIK |
| ROR1-1-scFv | 28 | DLQLQESGPGLVKPSQSLSLTCSVTGYSITSGYDWNWIRQFPGNKLEWMGYINY |
| | | DGSNNYNPSLKNRISITRDTSKNQFFLRLSSVTTEDTATYFCARGILRGYFDYWG |
| | | QGTTLTVSSGGGGSGGGGSGGGGSEIVLTQSPGSLAVSLGQRATISCRASESVDN |
| | | FGLSFMHWYQQKPGQPPKLLIYRASNLESGIPARFSGSGSRTDFTLTINPVETDDV |
| | | ATYYCQQSNKDPYTFGGGTKLEIK |
| ROR1-1-scFv- | 29 | DLQLQESGPGLVKPSQSLSLTCSVTGYSITSGYDWNWIRQFPGNKLEWMGYINY |
| hFc | | DGSNNYNPSLKNRISITRDTSKNQFFLRLSSVTTEDTATYFCARGILRGYFDYWG |
| | | QGTTLTVSSGGGGSGGGGSGGGGSEIVLTQSPGSLAVSLGQRATISCRASESVDN |
| | | FGLSFMHWYQQKPGQPPKLLIYRASNLESGIPARFSGSGSRTDFTLTINPVETDDV |
| | | ATYYCQQSNKDPYTFGGGTKLEIKEPKSSDKTHTCPPCPAPELLGGPSVFLFPPKP |
| | | KDTLMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGK |

TABLE 4
Sequences of CDR regions of ROR1 antibody

| | Numbering | | | |
|---|---|---|---|---|
| Antibody | scheme | CDR1 | CDR2 | CDR3 |
| ROR1-VH | Kabat | SGYDWN | YINYDGSNNYNPSLKN | GILRGYFDY |
| | | (SEQ ID NO: 30) | (SEQ ID NO: 31) | (SEQ ID NO: 32) |
| | Chothia | GYSITSGY | NYDGS | GILRGYFDY |
| | | (SEQ ID NO: 33) | (SEQ ID NO: 34) | (SEQ ID NO: 32) |
| | IMGT | GYSITSGYD | INYDGSN | ARGILRGYFDY |
| | | (SEQ ID NO: 35) | (SEQ ID NO: 36) | (SEQ ID NO: 37) |
| ROR1-VL | Kabat | RASESVDNFGLSFMH | RASNLES | QQSNKDPYT |
| | | (SEQ ID NO: 38) | (SEQ ID NO: 39) | (SEQ ID NO: 40) |
| | Chothia | RASESVDNFGLSFMH | RASNLES | QQSNKDPYT |
| | | (SEQ ID NO: 38) | (SEQ ID NO: 39) | (SEQ ID NO: 40) |
| | IMGT | ESVDNFGLSF | RAS | QQSNKDPYT |
| | | (SEQ ID NO: 41) | (SEQ ID NO: 42) | (SEQ ID NO: 40) |

### Example 2. Design of Chimeric Antigen Receptors in Different Forms

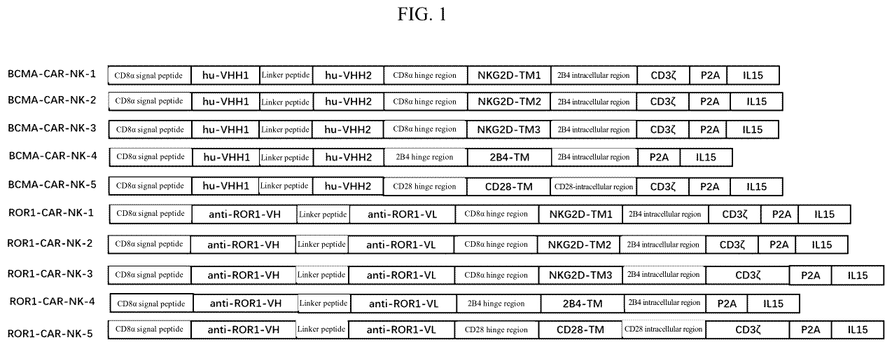

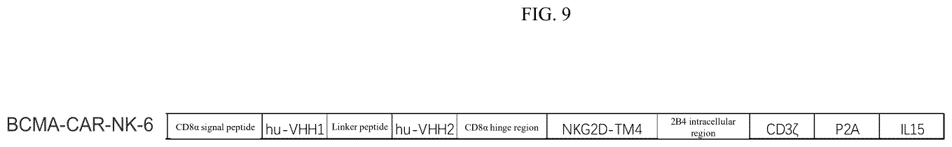

NKG2D TM1 was designed and loaded as a transmembrane region element into the chimeric antigen receptor to construct a novel chimeric antigen receptor. Chimeric antigen receptors loaded with other NKG2D transmembrane sequences and 2B4 or CD28 hinge region-transmembrane region and co-stimulatory domain were constructed, wherein the NKG2D-TM4 was derived from WO2021071962A1 (see SEQ ID No. 57 disclosed in WO2021071962A1 for details). In the subsequent examples, the differences in functionality (CAR positive rate and NK cell proliferation and killing) between CAR NK cells loaded with the NKG2D transmembrane region and those loaded with other transmembrane regions were evaluated, and the differences in functionality between CAR NK cells loaded with the NKG2D TM1 element and CAR NK cells loaded with other NKG2D TM sequences were also evaluated. The molecular structures of the chimeric antigen receptors are detailed in FIGS. 1 and 9, the sequences of the elements are detailed in Table 5, and the sequences of the CARs are detailed in Table 6. In FIGS. 1 and 9, BCMA-CAR-NK-1 is referred to hereinafter as BCMA-CAR1, BCMA-CAR-NK-2 is referred to hereinafter as BCMA-CAR2, BCMA-CAR-NK-3 is referred to hereinafter as BCMA-CAR3, BCMA-CAR-NK-4 is referred to hereinafter as BCMA-CAR4, BCMA-CAR-NK-5 is referred to hereinafter as BCMA-CAR5, and BCMA-CAR-NK-6 is referred to hereinafter as BCMA-CAR6; ROR1-CAR-NK-1 is referred to hereinafter as ROR1-CAR1, ROR2-CAR-NK-2 is referred to hereinafter as ROR1-CAR2, ROR1-CAR-NK-3 is referred to hereinafter as ROR1-CAR3, ROR1-CAR-NK-4 is referred to hereinafter as ROR1-CAR4, and ROR1-CAR-NK-5 is referred to hereinafter as ROR1-CAR5.

TABLE 5
Sequences of CAR elements

| NAME | SEQ ID NO: | SEQUENCE |
|---|---|---|
| CD8α signal peptide | 43 | MALPVTALLLPLALLLHAARP |
| CD28 SIGNAL PEPTIDE | 44 | MLRLLLALNLFPSIQVTG |
| CD8α hinge region | 45 | TTTPAPRPPTPAPTIASQPLSLRPEACRPAAGGAVHTRGLDFACD |
| 2B4 HINGE REGION | 46 | QDCQNAHQEFRFWP |
| CD28 HINGE REGION | 47 | EVMYPPPYLDNEKSNGTIIHVKGKHLCPSPLFPGPSKP |
| NKG2D-TM1 | 48 | SPFFFCCFIAVAMGIRFIIMVAIWSAVFLNS |
| NKG2D-TM2 | 49 | PFFFCCFIAVAMGIRFIIMVT |
| NKG2D-TM3 | 50 | PFFFCCFIAVAMGIRFIIMV |
| NKG2D-TM4 | 75 | SNLFVASWIAVMIIFRIGMAVAIFCCFFFPS |
| 2B4-TM | 51 | FLVIIVILSALFLGTLACFCV |
| CD28 TM | 52 | FWVLVVVGGVLACYSLLVTVAFIIFWV |
| 2B4 INTRACELLULAR | 53 | WRRKRKEKQSETSPKEFLTIYEDVKDLKTRRNHEQEQTFPGGG |
| REGION | | STIYSMIQSQSSAPTSQEPAYTLYSLIQPSRKSGSRKRNHSPSFNS |
| | | TIYEVIGKSQPKAQNPARLSRKELENFDVYS |
| CD28 INTRACELLULAR | 54 | RSKRSRLLHSDYMNMTPRRPGPTRKHYQPYAPPRDFAAYRS |
| REGION | | |
| CD3ζ | 55 | RVKFSRSADAPAYQQGQNQLYNELNLGRREEYDVLDKRRGRD |
| | | PEMGGKPRRKNPQEGLYNELQKDKMAEAYSEIGMKGERRRGK |
| | | GHDGLYQGLSTATKDTYDALHMQALPPR |
| P2A | 56 | ATNFSLLKQAGDVEENPGP |
| IL15 | 57 | MRISKPHLRSISIQCYLCLLLNSHFLTEAGIHVFILGCFSAGLPKT |
| | | EANWVNVISDLKKIEDLIQSMHID ATLYTESDVHPSCKVTAMKC |
| | | FLLELQVISLESGDASIHDTVENLIILANNSLSSNGNVTESGCKE |
| | | CEELEEKNIKEFLQSFVHIVQMFINTS |
| CD8α hinge | 58 | TTTPAPRPPTPAPTIASQPLSLRPEACRPAAGGAVHTRGLDFACD |
| region-NKG2D-TM1-2B4- | | SPFFFCCFIAVAMGIRFIIMVAIWSAVFLNSWRRKRKEKQSETSP |
| CD3ζ | | KEFLTIYEDVKDLKTRRNHEQEQTFPGGGSTIYSMIQSQSSAPTS |
| | | QEPAYTLYSLIQPSRKSGSRKRNHSPSFNSTIYEVIGKSQPKAQNP |
| | | ARLSRKELENFDVYSRVKFSRSADAPAYQQGQNQLYNELNLGR |
| | | REEYDVLDKRRGRDPEMGGKPRRKNPQEGLYNELQKDKMAE |
| | | AYSEIGMKGERRRGKGHDGLYQGLSTATKDTYDALHMQALPP |
| | | R |
| CD8α hinge | 59 | TTTPAPRPPTPAPTIASQPLSLRPEACRPAAGGAVHTRGLDFACD |
| region-NKG2D-TM1-2B4- | | SPFFFCCFIAVAMGIRFIIMVAIWSAVFLNSWRRKRKEKQSETSP |
| CD3ζ-P2A-IL15 | | KEFLTIYEDVKDLKTRRNHEQEQTFPGGGSTIYSMIQSQSSAPTS |
| | | QEPAYTLYSLIQPSRKSGSRKRNHSPSFNSTIYEVIGKSQPKAQNP |
| | | ARLSRKELENFDVYSRVKFSRSADAPAYQQGQNQLYNELNLGR |
| | | REEYDVLDKRRGRDPEMGGKPRRKNPQEGLYNELQKDKMAE |
| | | AYSEIGMKGERRRGKGHDGLYQGLSTATKDTYDALHMQALPP |
| | | RGSGATNFSLLKQAGDVEENPGPMRISKPHLRSISIQCYLCLLLN |
| | | SHFLTEAGIHVFILGCFSAGLPKTEANWVNVISDLKKIEDLIQSM |
| | | HIDATLYTESDVHPSCKVTAMKCFLLELQVISLESGDASIHDTVE |
| | | NLIILANNSLSSNGNVTESGCKECEELEEKNIKEFLQSFVHIVQM |
| | | FINTS |
| Hu-VHH1 | 60 | SEE TABLE 1, SEQ ID NO: 3, FOR DETAILS |
| Hu-VHH2 | 61 | SEE TABLE 1, SEQ ID NO: 4, FOR DETAILS |
| Linker1 | 62 | GGGGS |
| BCMA-Hu-VHH1-Linker1- | 63 | EVQLVESGGGLVQPGGSLRLSCSASGFTLDYYMIGWFRQAPGK |
| Hu-VHH2 | | EREGLSSISPADGSTYYADSVKGRFTISRDSSKNTVYLQMNSLR |
| | | AEDTAVYYCAAGNEATISWGFGPWEYDYWGQGTMVTVSSGG |
| | | GGSEVQLVESGGGLVQPGGSLRLSCAASESISSIHIMAWYRQAP |
| | | GKQRELVAGIRNDGSTVYVDSVKGRFTISRDNAKNSVYLQMNS |
| | | LRAEDTAVYYCNADQGFGSYSEWERRSRWGQGTTVTVSS |
| GFP | 64 | MVSKGEELFTGVVPILVELDGDVNGHKFSVRGEGEGDATNGKL |
| | | TLKFICTTGKLPVPWPTLVTTLTYGVQCFSRYPDHMKRHDFFKS |
| | | AMPEGYVQERTISFKDDGTYKTRAEVKFEGDTLVNRIELKGIDF |
| | | KEDGNILGHKLEYNFNSHNVYITADKQKNGIKANFKIRHNVED |
| | | GSVQLADHYQQNTPIGDGPVLLPDNHYLSTQSVLSKDPNEKRD |
| | | HMVLLEFVTAAGITHGMDELYK |
| ROR1-1-VH | 26 | SEE TABLE 3, SEQ ID NO: 26, FOR DETAILS |
| ROR1-1-VL | 27 | SEE TABLE 3, SEQ ID NO: 27, FOR DETAILS |
| ROR1-1-ScFv | 28 | SEE TABLE 3, SEQ ID NO: 28, FOR DETAILS |

TABLE 6
Sequences of CARs

| | SEQ ID | |
|---|---|---|
| NAME | NO: | SEQUENCE |
| BCMA-CAR1 | 65 | MALPVTALLLPLALLLHAARPEVQLVESGGGLVQPGGSLRLSCSASGF |
| (BCMA-CAR-NK-1) | | TLDYYMIGWFRQAPGKEREGLSSISPADGSTYYADSVKGRFTISRDSSK |
| | | NTVYLQMNSLRAEDTAVYYCAAGNEATISWGFGPWEYDYWGQGTM |
| | | VTVSSGGGGSEVQLVESGGGLVQPGGSLRLSCAASESISSIHIMAWYRQ |
| | | APGKQRELVAGIRNDGSTVYVDSVKGRFTISRDNAKNSVYLQMNSLR |
| | | AEDTAVYYCNADQGFGSYSEWERRSRWGQGTTVTVSSTTTPAPRPPTP |
| | | APTIASQPLSLRPEACRPAAGGAVHTRGLDFACDSPFFFCCFIAVAMGIR |
| | | FIIMVAIWSAVFLNSWRRKRKEKQSETSPKEFLTIYEDVKDLKTRRNHE |
| | | QEQTFPGGGSTIYSMIQSQSSAPTSQEPAYTLYSLIQPSRKSGSRKRNHS |
| | | PSFNSTIYEVIGKSQPKAQNPARLSRKELENFDVYSRVKFSRSADAPAY |
| | | QQGQNQLYNELNLGRREEYDVLDKRRGRDPEMGGKPRRKNPQEGLY |
| | | NELQKDKMAEAYSEIGMKGERRRGKGHDGLYQGLSTATKDTYDALH |
| | | MQALPPRGSGATNFSLLKQAGDVEENPGPMRISKPHLRSISIQCYLCLL |
| | | LNSHFLTEAGIHVFILGCFSAGLPKTEANWVNVISDLKKIEDLIQSMHID |
| | | ATLYTESDVHPSCKVTAMKCFLLELQVISLESGDASIHDTVENLIILANN |
| | | SLSSNGNVTESGCKECEELEEKNIKEFLQSFVHIVQMFINTS |
| BCMA-CAR2 | 66 | MALPVTALLLPLALLLHAARPEVQLVESGGGLVQPGGSLRLSCSASGF |
| (BCMA-CAR-NK-2) | | TLDYYMIGWFRQAPGKEREGLSSISPADGSTYYADSVKGRFTISRDSSK |
| | | NTVYLQMNSLRAEDTAVYYCAAGNEATISWGFGPWEYDYWGQGTM |
| | | VTVSSGGGGSEVQLVESGGGLVQPGGSLRLSCAASESISSIHIMAWYRQ |
| | | APGKQRELVAGIRNDGSTVYVDSVKGRFTISRDNAKNSVYLQMNSLR |
| | | AEDTAVYYCNADQGFGSYSEWERRSRWGQGTTVTVSSTTTPAPRPPTP |
| | | APTIASQPLSLRPEACRPAAGGAVHTRGLDFACDPFFFCCFIAVAMGIRF |
| | | IIMVTWRRKRKEKQSETSPKEFLTIYEDVKDLKTRRNHEQEQTFPGGG |
| | | STIYSMIQSQSSAPTSQEPAYTLYSLIQPSRKSGSRKRNHSPSFNSTIYEVI |
| | | GKSQPKAQNPARLSRKELENFDVYSRVKFSRSADAPAYQQGQNQLYN |
| | | ELNLGRREEYDVLDKRRGRDPEMGGKPRRKNPQEGLYNELQKDKMA |
| | | EAYSEIGMKGERRRGKGHDGLYQGLSTATKDTYDALHMQALPPRGSG |
| | | ATNFSLLKQAGDVEENPGPMRISKPHLRSISIQCYLCLLLNSHFLTEAGI |
| | | HVFILGCFSAGLPKTEANWVNVISDLKKIEDLIQSMHIDATLYTESDVH |
| | | PSCKVTAMKCFLLELQVISLESGDASIHDTVENLIILANNSLSSNGNVTE |
| | | SGCKECEELEEKNIKEFLQSFVHIVQMFINTS |
| BCMA-CAR3 | 67 | MALPVTALLLPLALLLHAARPEVQLVESGGGLVQPGGSLRLSCSASGF |
| (BCMA-CAR-NK-3) | | TLDYYMIGWFRQAPGKEREGLSSISPADGSTYYADSVKGRFTISRDSSK |
| | | NTVYLQMNSLRAEDTAVYYCAAGNEATISWGFGPWEYDYWGQGTM |
| | | VTVSSGGGGSEVQLVESGGGLVQPGGSLRLSCAASESISSIHIMAWYRQ |
| | | APGKQRELVAGIRNDGSTVYVDSVKGRFTISRDNAKNSVYLQMNSLR |
| | | AEDTAVYYCNADQGFGSYSEWERRSRWGQGTTVTVSSTTTPAPRPPTP |
| | | APTIASQPLSLRPEACRPAAGGAVHTRGLDFACDPFFFCCFIAVAMGIRF |
| | | IIMVWRRKRKEKQSETSPKEFLTIYEDVKDLKTRRNHEQEQTFPGGGS |
| | | TIYSMIQSQSSAPTSQEPAYTLYSLIQPSRKSGSRKRNHSPSFNSTIYEVI |
| | | GKSQPKAQNPARLSRKELENFDVYSRVKFSRSADAPAYQQGQNQLYN |
| | | ELNLGRREEYDVLDKRRGRDPEMGGKPRRKNPQEGLYNELQKDKMA |
| | | EAYSEIGMKGERRRGKGHDGLYQGLSTATKDTYDALHMQALPPRGSG |
| | | ATNFSLLKQAGDVEENPGPMRISKPHLRSISIQCYLCLLLNSHFLTEAGI |
| | | HVFILGCFSAGLPKTEANWVNVISDLKKIEDLIQSMHIDATLYTESDVH |
| | | PSCKVTAMKCFLLELQVISLESGDASIHDTVENLIILANNSLSSNGNVTE |
| | | SGCKECEELEEKNIKEFLQSFVHIVQMFINTS |
| BCMA-CAR4 | 68 | MALPVTALLLPLALLLHAARPEVQLVESGGGLVQPGGSLRLSCSASGF |
| (BCMA-CAR-NK-4) | | TLDYYMIGWFRQAPGKEREGLSSISPADGSTYYADSVKGRFTISRDSSK |
| | | NTVYLQMNSLRAEDTAVYYCAAGNEATISWGFGPWEYDYWGQGTM |
| | | VTVSSGGGGSEVQLVESGGGLVQPGGSLRLSCAASESISSIHIMAWYRQ |
| | | APGKQRELVAGIRNDGSTVYVDSVKGRFTISRDNAKNSVYLQMNSLR |
| | | AEDTAVYYCNADQGFGSYSEWERRSRWGQGTTVTVSSQDCQNAHQE |
| | | FRFWPFLVIIVILSALFLGTLACFCVWRRKRKEKQSETSPKEFLTIYEDV |
| | | KDLKTRRNHEQEQTFPGGGSTIYSMIQSQSSAPTSQEPAYTLYSLIQPSR |
| | | KSGSRKRNHSPSFNSTIYEVIGKSQPKAQNPARLSRKELENFDVYSGSG |
| | | ATNFSLLKQAGDVEENPGPMRISKPHLRSISIQCYLCLLLNSHFLTEAGI |
| | | HVFILGCFSAGLPKTEANWVNVISDLKKIEDLIQSMHIDATLYTESDVH |
| | | PSCKVTAMKCFLLELQVISLESGDASIHDTVENLIILANNSLSSNGNVTE |
| | | SGCKECEELEEKNIKEFLQSFVHIVQMFINTS |
| BCMA-CAR5 | 69 | MALPVTALLLPLALLLHAARPEVQLVESGGGLVQPGGSLRLSCSASGF |
| (BCMA-CAR-NK-5) | | TLDYYMIGWFRQAPGKEREGLSSISPADGSTYYADSVKGRFTISRDSSK |
| | | NTVYLQMNSLRAEDTAVYYCAAGNEATISWGFGPWEYDYWGQGTM |
| | | VTVSSGGGGSEVQLVESGGGLVQPGGSLRLSCAASESISSIHIMAWYRQ |
| | | APGKQRELVAGIRNDGSTVYVDSVKGRFTISRDNAKNSVYLQMNSLR |
| | | AEDTAVYYCNADQGFGSYSEWERRSRWGQGTTVTVSSAAAIEVMYPP |
| | | PYLDNEKSNGTIIHVKGKHLCPSPLFPGPSKPFWVLVVVGGVLACYSL |
| | | LVTVAFIIFWVRSKRSRLLHSDYMNMTPRRPGPTRKHYQPYAPPRDFA |
| | | AYRSRVKFSRSADAPAYQQGQNQLYNELNLGRREEYDVLDKRRGRDP |
| | | EMGGKPRRKNPQEGLYNELQKDKMAEAYSEIGMKGERRRGKGHDGL |
| | | YQGLSTATKDTYDALHMQALPPRGSGATNFSLLKQAGDVEENPGPMR |
| | | ISKPHLRSISIQCYLCLLLNSHFLTEAGIHVFILGCFSAGLPKTEANWVN |
| | | VISDLKKIEDLIQSMHIDATLYTESDVHPSCKVTAMKCFLLELQVISLES |
| | | GDASIHDTVENLIILANNSLSSNGNVTESGCKECEELEEKNIKEFLQSFV |
| | | HIVQMFINTS |
| BCMA-CAR6 | 76 | MALPVTALLLPLALLLHAARPEVQLVESGGGLVQPGGSLRLSCSASGF |
| (BCMA-CAR-NK-6) | | TLDYYMIGWFRQAPGKEREGLSSISPADGSTYYADSVKGRFTISRDSSK |
| | | NTVYLQMNSLRAEDTAVYYCAAGNEATISWGFGPWEYDYWGQGTM |
| | | VTVSSGGGGSEVQLVESGGGLVQPGGSLRLSCAASESISSIHIMAWYRQ |
| | | APGKQRELVAGIRNDGSTVYVDSVKGRFTISRDNAKNSVYLQMNSLR |
| | | AEDTAVYYCNADQGFGSYSEWERRSRWGQGTTVTVSSTTTPAPRPPTP |
| | | APTIASQPLSLRPEACRPAAGGAVHTRGLDFACDSNLFVASWIAVMIIFR |
| | | IGMAVAIFCCFFFPSWRRKRKEKQSETSPKEFLTIYEDVKDLKTRRNHE |
| | | QEQTFPGGGSTIYSMIQSQSSAPTSQEPAYTLYSLIQPSRKSGSRKRNHS |
| | | PSFNSTIYEVIGKSQPKAQNPARLSRKELENFDVYSRVKFSRSADAPAY |
| | | QQGQNQLYNELNLGRREEYDVLDKRRGRDPEMGGKPRRKNPQEGLY |
| | | NELQKDKMAEAYSEIGMKGERRRGKGHDGLYQGLSTATKDTYDALH |
| | | MQALPPRGSGATNFSLLKQAGDVEENPGPMRISKPHLRSISIQCYLCLL |
| | | LNSHFLTEAGIHVFILGCFSAGLPKTEANWVNVISDLKKIEDLIQSMHID |
| | | ATLYTESDVHPSCKVTAMKCFLLELQVISLESGDASIHDTVENLIILANN |
| | | SLSSNGNVTESGCKECEELEEKNIKEFLQSFVHIVQMFINTS |
| ROR1-CAR1 | 70 | MALPVTALLLPLALLLHAARPDLQLQESGPGLVKPSQSLSLTCSVTGYS |
| (ROR1-CAR-NK-1) | | ITSGYDWNWIRQFPGNKLEWMGYINYDGSNNYNPSLKNRISITRDTSK |
| | | NQFFLRLSSVTTEDTATYFCARGILRGYFDYWGQGTTLTVSSGGGGSG |
| | | GGGSGGGGSEIVLTQSPGSLAVSLGQRATISCRASESVDNFGLSFMHW |
| | | YQQKPGQPPKLLIYRASNLESGIPARFSGSGSRTDFTLTINPVETDDVAT |
| | | YYCQQSNKDPYTFGGGTKLEIKTTTPAPRPPTPAPTIASQPLSLRPEACR |
| | | PAAGGAVHTRGLDFACDSPFFFCCFIAVAMGIRFIIMVAIWSAVFLNSWR |
| | | RKRKEKQSETSPKEFLTIYEDVKDLKTRRNHEQEQTFPGGGSTIYSMIQ |
| | | SQSSAPTSQEPAYTLYSLIQPSRKSGSRKRNHSPSFNSTIYEVIGKSQPKA |
| | | QNPARLSRKELENFDVYSRVKFSRSADAPAYQQGQNQLYNELNLGRR |
| | | EEYDVLDKRRGRDPEMGGKPRRKNPQEGLYNELQKDKMAEAYSEIG |
| | | MKGERRRGKGHDGLYQGLSTATKDTYDALHMQALPPRGSGATNFSLL |
| | | KQAGDVEENPGPMRISKPHLRSISIQCYLCLLLNSHFLTEAGIHVFILGC |
| | | FSAGLPKTEANWVNVISDLKKIEDLIQSMHIDATLYTESDVHPSCKVTA |
| | | MKCFLLELQVISLESGDASIHDTVENLIILANNSLSSNGNVTESGCKEC |
| | | EELEEKNIKEFLQSFVHIVQMFINTS |
| ROR1-CAR2 | 71 | MALPVTALLLPLALLLHAARPDLQLQESGPGLVKPSQSLSLTCSVTGYS |
| (ROR1-CAR-NK-2) | | ITSGYDWNWIRQFPGNKLEWMGYINYDGSNNYNPSLKNRISITRDTSK |
| | | NQFFLRLSSVTTEDTATYFCARGILRGYFDYWGQGTTLTVSSGGGGSG |
| | | GGGSGGGGSEIVLTQSPGSLAVSLGQRATISCRASESVDNFGLSFMHW |
| | | YQQKPGQPPKLLIYRASNLESGIPARFSGSGSRTDFTLTINPVETDDVAT |
| | | YYCQQSNKDPYTFGGGTKLEIKTTTPAPRPPTPAPTIASQPLSLRPEACR |
| | | PAAGGAVHTRGLDFACDPFFFCCFIAVAMGIRFIIMVTWRRKRKEKQSE |
| | | TSPKEFLTIYEDVKDLKTRRNHEQEQTFPGGGSTIYSMIQSQSSAPTSQE |
| | | PAYTLYSLIQPSRKSGSRKRNHSPSFNSTIYEVIGKSQPKAQNPARLSRK |
| | | ELENFDVYSRVKFSRSADAPAYQQGQNQLYNELNLGRREEYDVLDKR |
| | | RGRDPEMGGKPRRKNPQEGLYNELQKDKMAEAYSEIGMKGERRRGK |
| | | GHDGLYQGLSTATKDTYDALHMQALPPRGSGATNFSLLKQAGDVEEN |
| | | PGPMRISKPHLRSISIQCYLCLLLNSHFLTEAGIHVFILGCFSAGLPKTEA |
| | | NWVNVISDLKKIEDLIQSMHIDATLYTESDVHPSCKVTAMKCFLLELQ |
| | | VISLESGDASIHDTVENLIILANNSLSSNGNVTESGCKECEELEEKNIKE |
| | | FLQSFVHIVQMFINTS |
| ROR1-CAR3 | 72 | MALPVTALLLPLALLLHAARPDLQLQESGPGLVKPSQSLSLTCSVTGYS |
| (ROR1-CAR-NK-3) | | ITSGYDWNWIRQFPGNKLEWMGYINYDGSNNYNPSLKNRISITRDTSK |
| | | NQFFLRLSSVTTEDTATYFCARGILRGYFDYWGQGTTLTVSSGGGGSG |
| | | GGGSGGGGSEIVLTQSPGSLAVSLGQRATISCRASESVDNFGLSFMHW |
| | | YQQKPGQPPKLLIYRASNLESGIPARFSGSGSRTDFTLTINPVETDDVAT |
| | | YYCQQSNKDPYTFGGGTKLEIKTTTPAPRPPTPAPTIASQPLSLRPEACR |
| | | PAAGGAVHTRGLDFACDPFFFCCFIAVAMGIRFIIMVWRRKRKEKQSET |
| | | SPKEFLTIYEDVKDLKTRRNHEQEQTFPGGGSTIYSMIQSQSSAPTSQEP |
| | | AYTLYSLIQPSRKSGSRKRNHSPSFNSTIYEVIGKSQPKAQNPARLSRKE |
| | | LENFDVYSRVKFSRSADAPAYQQGQNQLYNELNLGRREEYDVLDKRR |
| | | GRDPEMGGKPRRKNPQEGLYNELQKDKMAEAYSEIGMKGERRRGKG |
| | | HDGLYQGLSTATKDTYDALHMQALPPRGSGATNFSLLKQAGDVEENP |
| | | GPMRISKPHLRSISIQCYLCLLLNSHFLTEAGIHVFILGCFSAGLPKTEA |
| | | NWVNVISDLKKIEDLIQSMHIDATLYTESDVHPSCKVTAMKCFLLELQ |
| | | VISLESGDASIHDTVENLIILANNSLSSNGNVTESGCKECEELEEKNIKE |
| | | FLQSFVHIVQMFINTS |
| ROR1-CAR4 | 73 | MALPVTALLLPLALLLHAARPDLQLQESGPGLVKPSQSLSLTCSVTGYS |
| (ROR1-CAR-NK-4) | | ITSGYDWNWIRQFPGNKLEWMGYINYDGSNNYNPSLKNRISITRDTSK |
| | | NQFFLRLSSVTTEDTATYFCARGILRGYFDYWGQGTTLTVSSGGGGSG |
| | | GGGSGGGGSEIVLTQSPGSLAVSLGQRATISCRASESVDNFGLSFMHW |
| | | YQQKPGQPPKLLIYRASNLESGIPARFSGSGSRTDFTLTINPVETDDVAT |
| | | YYCQQSNKDPYTFGGGTKLEIKQDCQNAHQEFRFWPFLVIIVILSALFL |
| | | GTLACFCVWRRKRKEKQSETSPKEFLTIYEDVKDLKTRRNHEQEQTFP |
| | | GGGSTIYSMIQSQSSAPTSQEPAYTLYSLIQPSRKSGSRKRNHSPSFNSTI |
| | | YEVIGKSQPKAQNPARLSRKELENFDVYSGSGATNFSLLKQAGDVEEN |
| | | PGPMRISKPHLRSISIQCYLCLLLNSHFLTEAGIHVFILGCFSAGLPKTEA |
| | | NWVNVISDLKKIEDLIQSMHIDATLYTESDVHPSCKVTAMKCFLLELQ |
| | | VISLESGDASIHDTVENLIILANNSLSSNGNVTESGCKECEELEEKNIKE |
| | | FLQSFVHIVQMFINTS |
| ROR1-CAR5 | 74 | MALPVTALLLPLALLLHAARPDLQLQESGPGLVKPSQSLSLTCSVTGYS |
| (ROR1-CAR-NK-5) | | ITSGYDWNWIRQFPGNKLEWMGYINYDGSNNYNPSLKNRISITRDTSK |
| | | NQFFLRLSSVTTEDTATYFCARGILRGYFDYWGQGTTLTVSSGGGGSG |
| | | GGGSGGGGSEIVLTQSPGSLAVSLGQRATISCRASESVDNFGLSFMHW |
| | | YQQKPGQPPKLLIYRASNLESGIPARFSGSGSRTDFTLTINPVETDDVAT |
| | | YYCQQSNKDPYTFGGGTKLEIKAAAIEVMYPPPYLDNEKSNGTIIHVK |
| | | GKHLCPSPLFPGPSKPFWVLVVVGGVLACYSLLVTVAFIIFWVRSKRSR |
| | | LLHSDYMNMTPRRPGPTRKHYQPYAPPRDFAAYRSRVKFSRSADAPAY |
| | | QQGQNQLYNELNLGRREEYDVLDKRRGRDPEMGGKPRRKNPQEGLY |
| | | NELQKDKMAEAYSEIGMKGERRRGKGHDGLYQGLSTATKDTYDALH |
| | | MQALPPRGSGATNFSLLKQAGDVEENPGPMRISKPHLRSISIQCYLCLL |
| | | LNSHFLTEAGIHVFILGCFSAGLPKTEANWVNVISDLKKIEDLIQSMHID |
| | | ATLYTESDVHPSCKVTAMKCFLLELQVISLESGDASIHDTVENLIILANN |
| | | SLSSNGNVTESGCKECEELEEKNIKEFLQSFVHIVQMFINTS |

### Example 3. Preparation of Chimeric Antigen Receptor Retroviruses

The nucleic acid sequences encoding the CARs described in Example 2 (Table 6) were loaded into a retroviral vector using conventional molecular biology methods in the art to construct target plasmids. The day before virus packaging, 293T cells (purchased from ATCC) were trypsinized and inoculated into a culture dish at 1E7 cells/10 cm. When the cells were transfected, the packaging plasmids and the target plasmids were mixed and then added to an α-MEM culture medium, and the FuGENE® HD transfection reagent (Promega, E2311) was added to another centrifuge tube containing the α-MEM culture medium. The diluted transfection reagent was added dropwise above the diluted plasmid, and the mixture was well mixed, and left to stand at room temperature for 15 min. Finally, the mixture of plasmid and the transfection reagent was added to a 10 cm culture dish, gently shaken 10 times to mix well, and put into an incubator. Three days after the cell transfection, the viruses were harvested, and 10 mL of virus-containing culture supernatant was transferred to a 50-mL centrifuge tube and centrifuged at 1250 rpm at 4° C. for 5 min to remove dead 293T cells. The virus-containing supernatant was filtered, concentrated using the Retro-X Concentrator reagent (Clontech, 631455), and after packaging, stored at −80° C. for later use.

### Example 4. Preparation of CAR-NK

### 1. Isolation and Activation of NK Lymphocytes

Fresh PBMCs were centrifuged at 500 g at room temperature for 7 min, the culture medium supernatant was discarded, and then NK cells were isolated using the Human NK Cell isolation kit (Stemcell, 17955). The isolated NK cells were activated using K562 cells, and the activation procedures are as follows: On day 0, the cells were counted by AO/PI and mixed in a ratio of NK:K562=1:2. The mixed cells were added to a Non-Treated 6-well plate at 2 mL/well (the culture medium was an NK cell culture medium containing 200 IU/mL of human IL2 (Miltenyi Biotec, 130-114-429)), and cultured in an incubator (37° C., 5% CO₂). On day 4, 3 mL of the culture medium was added to each well. On day 6, the cell activation was completed and the transfection could be performed.

### 2. Retroviral Infection of NK Cells

On day 1, a 24-well plate was coated with the RetroNectin reagent (Takara, T202) at a concentration of 7 μg/mL at 500 μL/well, and incubated at 4° C. overnight. On day 2, the RetroNectin supernatant was discarded, and the plate was washed with PBS once. The NK cells were infected at a MOI=5, the amount of viruses was calculated according to the virus titer, and the viruses were added to a 24-well plate. The 24-well plate to which the viruses had been added was centrifuged at 2000 g at 4-8° C. for 60 min. The virus fluid supernatant was discarded. The NK cells were counted and added to the 24-well plate at 3E5 cells/well, and the mixture was centrifuged at 400 g at room temperature for 5 min. The 24-well plate was cultured in an incubator (37° C., 5% CO₂). On day 3, the transfected NK cells were transferred into a Non-Treated 6-well plate. On day 6, the CAR expression and CAR NK cell proliferation were measured.

### Example 5. Assays of Expression Efficiency of Chimeric Antigen Receptors in Different Forms and Cell Proliferation Efficiency

On day 6 after the retroviral infection of NK cells, the expression rate of CARs on the surface of the NK cell membrane was determined by flow cytometry, and the CAR NK cell proliferation was detected using a cell counter.

### 1. Assay of CAR Expression Rate in NK Cells

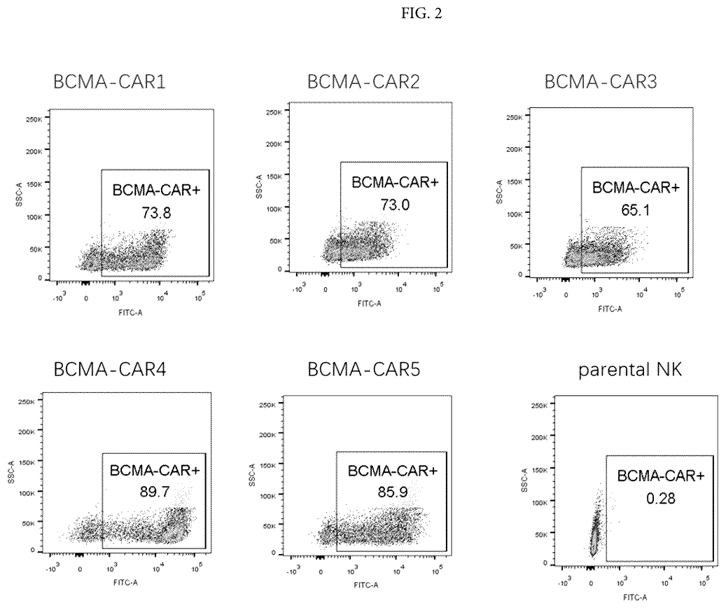

(1) BCMA: 2E5 cells were added to a 96-well U-shaped plate and centrifuged, and the supernatant was discarded. The cells were washed with a buffer, and then 100 μL FITC-hBCMA-his (ACROBiosystems, BCA-HF254) was added at a final concentration of 2 μg/mL. The mixture was incubated at 4° C. in the dark for 1 h. After the incubation was completed, the mixture was centrifuged, and the supernatant was discarded. The cells were washed with a buffer and then resuspended. The BCMA-CAR expression rate was determined on a BD FACS Canto II flow cytometer. The flow cytometry results of the molecule expression on the surface of the BCMA-CAR NK cells are shown in FIG. 2. The results show that the expression efficiency of the BCMA-CAR in NK cells was about 70-90%, and the descending expression efficiency order was BCMA-CAR4 (89.7%)>BCMA-CAR5 (85.9%)>BCMA-CAR1 (73.8%)>BCMA-CAR2 (73%)>BCMA-CAR3 (65.1%).

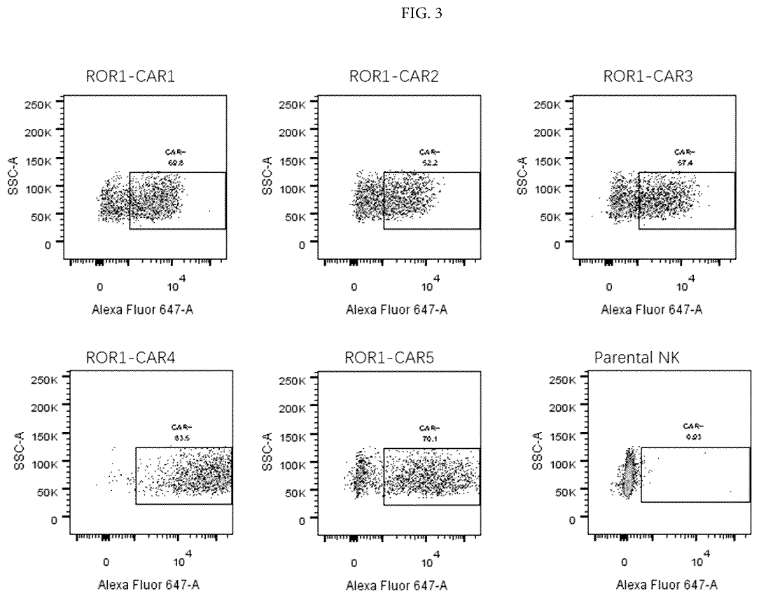

(2) ROR1: 2E5 cells were added to a 96-well U-shaped plate and centrifuged, and the supernatant was discarded. The cells were washed with a buffer, and then 100 μL of human ROR1-his (ACROBiosystems, RO1-H522y) at a final concentration of 10 μg/mL was added. The mixture was incubated at 4° C. in the dark for 1 h. After the incubation was completed, the mixture was centrifuged, and the supernatant was discarded. The cells were washed with a buffer, and then 100 μL of THE™ His Tag Antibody [iFluor 647] (GenScript, A01802) at a final concentration of 1 μg/mL was added. The mixture was incubated at 4° C. in the dark for 1 h. After the incubation was completed, the mixture was centrifuged, and the supernatant was discarded. The cells were washed with a buffer, and then resuspended. The ROR1-CAR expression rate was detected on a BD FACS Canto II flow cytometer. The flow cytometry results of the molecule expression on the surface of the ROR1 CAR NK cells are shown in FIG. 3. The results show that the expression efficiency of ROR1 CAR in NK cells was about 50-80%, and the descending expression efficiency order was ROR1-CAR4 (83.5%)>ROR1-CAR5 (70.1%)>ROR1-CAR1 (69.8%)>ROR1-CAR3 (57.4%)>ROR1-CAR2 (52.2%).

### 2. Determination of Proliferation Rate of CAR NK Cells

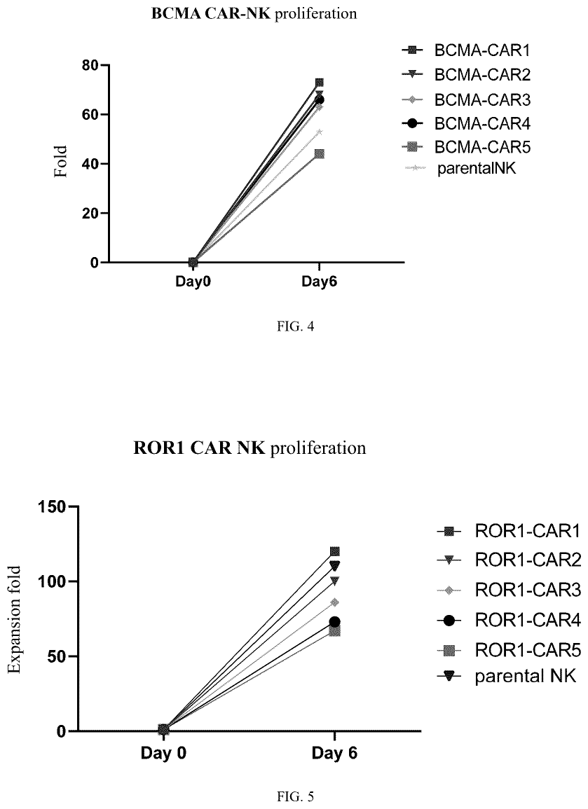

The proliferation rates of the BCMA-CAR NK cells and the ROR1 CAR NK cells were determined by AO/PI counting, and the results are shown in FIGS. 4 and 5. The BCMA-CAR NK cells were expanded by about 40-70 folds 6 days after the transfection, and the descending proliferation rate order was BCMA-CAR1 (73 folds)>BCMA-CAR2 (68 folds)>BCMA-CAR4 (66 folds)>BCMA-CAR3 (63 folds)>parental NK (53 folds)>BCMA-CAR5 (44 folds). The ROR1-CAR NK cells were expanded by about 60-120 folds 6 days after the transfection, and the descending proliferation rate order was ROR1-CAR1 (120 folds)>parental NK (110 folds)>ROR1-CAR2 (100 folds)>ROR1-CAR3 (86 folds)>ROR1-CAR4 (73 folds)>ROR1-CAR5 (67 folds).

The results show that:

As can be seen from the above results, in terms of the expression rate of CAR, the expression rate of CARs in the NK cells loaded with the NKG2D TM element was lower than those of the CARs in other forms; the expression rate of CAR loaded with the NKG2D-TM1 element (CAR1) was higher than those of CARs loaded with the NKG2D-TM2 element or NKG2D-TM3 element (CAR2 and CAR3). However, the proliferation rate of CAR NK cells loaded with the NKG2D TM element was generally higher than that of the CAR NK cells using other transmembrane elements, wherein the proliferation rate of CAR NK cells loaded with the NKG2D-TM1 element was the highest and higher than those of the CAR NK cells loaded with other transmembrane elements or other NKG2D TM sequences.

### Example 6. 4 h In Vitro Killing Function Assessment of CAR NK in Different Forms

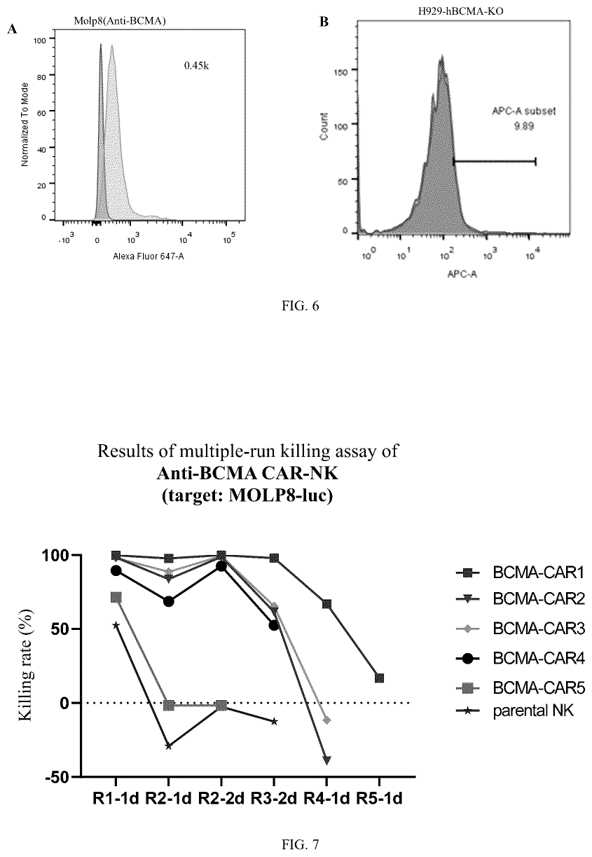

The BCMA expression in multiple myeloma MOLP8 cells and H929-hBCMA-KO cells was detected using flow cytometry. The results are shown in FIG. 6.

On day 6 after the retroviral infection, NK cells were subjected to a 4 h in vitro killing assay: The MOLP8 or H929-hBCMA-KO target cells diluted with a 1640 culture medium were added to a white opaque 96-well plate at 2E4 cells/50 μL/well, and the NK cells were added to the target cells in effector-to-target ratios of 10:1, 5:1, and 2.5:1. The 96-well plate was incubated in an incubator at 37° C. with 5% CO₂, and after incubation for 4 h, 30 μL of FIREFLYGLO luciferase reporter gene detection reagent (MeilunBio®, MA0519-1) was added. The mixture was incubated at room temperature in the dark for 10 min and detected on a microplate reader. The killing rate was calculated.

The 4 h in vitro cell killing effect on MOLP8 cells is detailed in Table 7. Compared with other CAR structures, the CAR structures loaded with NKG2D TM region generally showed stronger tumor cell killing function in effector-to-target ratios of 10:1, 5:1, and 2.5:1, and the CAR loaded with NKG2D TM1 (BCMA-CAR1) had the strongest killing function, which was superior to NKG2D TM in other forms. In addition, the killing effects of the NK cells transfected with the BCMA-CARs 1-5 were basically equivalent to that of the parental NK on H929-hBCMA-KO cells, indicating that the BCMA-CAR had no non-specific killing effect on the H929-hBCMA-KO cells.

TABLE 7
4 h in vitro killing rate (%) of
BCMA-CAR NK against MOLP8 cells

| | E:T | | |
|---|---|---|---|
| | 10:1 | 5:1 | 2.5:1 |
| BCMA-CAR1 | 81.45 | 67.59 | 43.59 |
| BCMA-CAR2 | 60.72 | 32.45 | 27.55 |
| BCMA-CAR3 | 63.69 | 49.93 | 16.57 |
| BCMA-CAR4 | 50.67 | 46.36 | 10.91 |
| BCMA-CAR5 | 49.06 | 21.36 | 35.32 |
| Parental NK | 60.62 | 20.62 | 14.96 |

Note:
ND denotes “no killing”

### Example 7. Function Assessment of CAR NK in Different Forms by Multiple-Run Killing Assay

### 1. Multiple-Run Killing Assay of BCMA-CAR NK Cells

On day 6 after the retroviral infection of NK cells, the multiple-run in vitro killing effects of BCMA-CAR NK cells on MOLP8 cells were detected: (1) First run of killing: The MOLP8 target cells diluted with a 1640 culture medium were added to a 12-well plate at 2.5E5 cells/500 μL/well. The NK cells were added to the 12-well plate in an effector-to-target ratio of 1:1, and the mixture was cultured in an incubator at 37° C. with 5% CO₂ for 24 h. After incubation for 24 h, 100 μL of well-mixed cells described above was added to a white opaque 96-well plate, and 30 μL of FIREFLYGLO luciferase reporter gene detection reagent (MeilunBio®, MA0519-1) was added. The mixture was incubated at room temperature in the dark for 10 min, and then the fluorescence intensity was measured using a microplate reader. The NK cell killing efficiency was calculated. Subsequently, the next run of killing assay was performed directly or after the NK cell killing rate was measured again upon incubation for another 24 h. (2) The next run of killing: The cells on the 12-well plate in the previous run were taken, and the NK cells were counted. The NK cells in the previous run were added to a 12-well plate inoculated with new target cells in an effector-to-target ratio of 1:1, and step (1) was repeated. The NK cell killing rate was measured and the next run of killing assay continued.

The results for the multiple-run killing assay of BCMA-CAR NK cells are shown in FIG. 7. As shown in FIG. 7, the killing effect of BCMA-CAR loaded with the NKG2D TM element was superior to those of the BCMA-CARs in other forms, wherein BCMA-CAR1 loaded with the NKG2D-TM1 had the best killing effect superior to those of the BCMA-CARs in other forms and the CARs loaded with other NKG2D-TMs, and could still maintain a 98% cell killing rate in the third run of killing (R3-2d).

### 2. Multiple-Run Killing Assay of ROR1-CAR NK Cells

On day 6 after the retroviral infection of NK cells, the multiple-run in vitro killing effects of ROR1 CAR NK cells on A549 cells (with low ROR1 expression), 786-O cells (with low ROR1 expression), and RPMI 8226 cells (with medium ROR1 expression) were detected: The A549, 786-O, or RPMI 8226 target cells diluted with a 1640 culture medium were added to a white opaque 96-well plate at 2.0E4 cells/50 L/well, and the NK cells were added to the 96-well plate in an effector-to-target ratio of 1:1. The plate was incubated in an incubator at 37° C. with 5% CO₂ for 24 h, and after incubation for 24 h, 30 μL of FIREFLYGLO luciferase reporter gene detection reagent (MeilunBio®, MA0519-1) was added. The mixture was incubated at room temperature in the dark for 10 min, and then the fluorescence value was measured using a microplate reader. The NK cell killing efficiency was calculated. The NK cells on the 96-well plate in the previous run were counted and were added to another 96-well plate containing new target cells in an effector-to-target ratio of 1:1. The procedures described above were repeated.

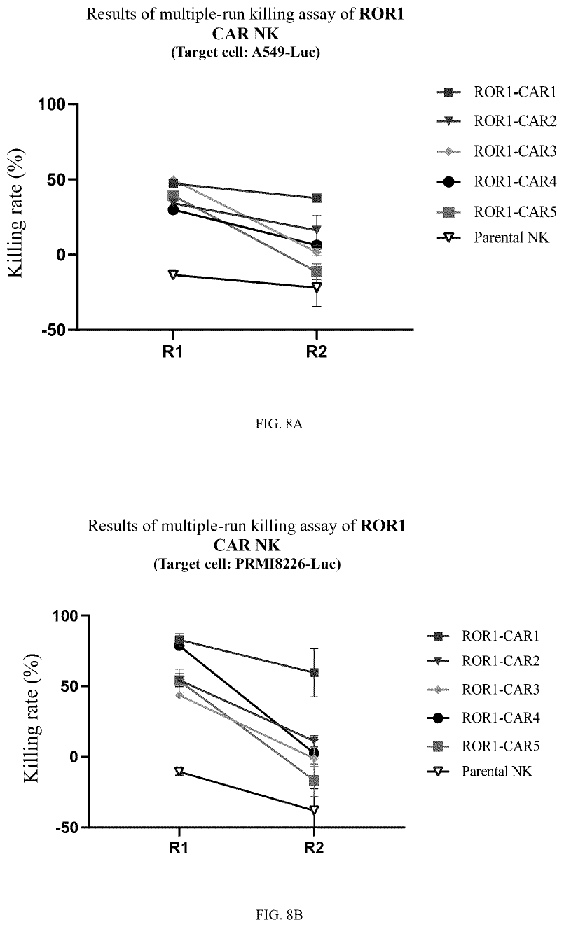

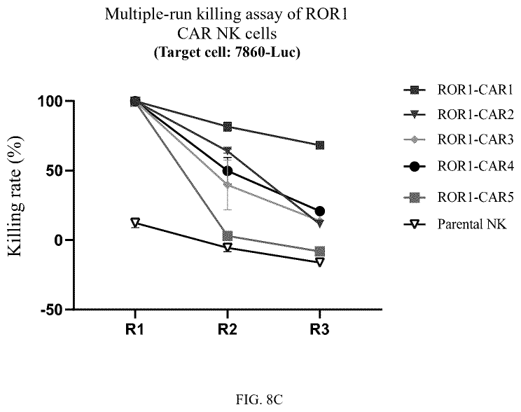

The results for the multiple-run killing assay of ROR1-CAR NK cells are shown in FIGS. 8A-8C. The results of the second and third runs of killing show that the ROR-1 CAR1 NK cells had better killing effects on 786-O cells, A549 cells, and RPMI 8226 cells than those of CARs without NKG2D-TM and CARs with other types of NKG2D-TM.

### Example 8. Cytokine Release Functional Assessment of CAR NK in Different Forms

After transfected with BCMA-CAR, the NK cells were cultured for 14 days, and then co-incubated with MOLP8 cells in effector-to-target ratios of 10:1 and 2.5:1 for 24 h. The IFN-γ content in the collected supernatant was detected according to the instructions of the human IFN-γ quantitative ELISA kit (BD, 555142). The content of IFN-γ in the supernatant of the test samples was calculated according to the standard curve of the standards, and the results are shown in Table 8.

After transfected with ROR1-CAR, the NK cells were cultured for 14 days, and then co-incubated with RPMI 8226 cells in effector-to-target ratios of 1:1 and 1:3 for 24 h. Then, the IFN-γ content in the collected supernatant was detected using the human IFN-γ quantitative ELISA kit (BD, 555142), and the results are shown in Table 9.

The results in Tables 8 and 9 show that the CAR1 can up-regulate the expression level of the cytokine IFN-γ with tumor-killing effect in different effector-to-target ratios for different target cells, with a superior effect to those of the CARs without NKG2D elements and the CARs with other NKG2D-TM elements.

TABLE 8
Detection results of cytokine IFN-γ content

IFN-γ content (pg/mL)

| | E:T = 10:1 | E:T = 2.5:1 |
|---|---|---|
| BCMA-CAR1 | 1721.356 | 1530.961 |
| BCMA-CAR2 | 964.3112 | ND |
| BCMA-CAR3 | ND | ND |
| BCMA-CAR4 | ND | ND |
| BCMA-CAR5 | 610.7216 | ND |
| Parental NK | 424.8604 | ND |

Note:
ND denotes “not detected”

TABLE 9
Detection results of cytokine IFN-γ content (pg/mL)

IFN-γ content (pg/mL)

| | E:T = 1:1 | E:T = 1:3 |
|---|---|---|
| ROR1-CAR1 | 2557.26 | 1352.652 |
| ROR1-CAR2 | 482.656 | ND |
| ROR1-CAR3 | 387.052 | ND |
| ROR1-CAR4 | 1916.72 | 1594.85 |
| ROR1-CAR5 | 394.425 | 412.546 |
| Parental NK | ND | ND |

Note:
ND denotes “not detected”

### Example 9. In Vivo Efficacy Assessment of BCMA-BCMA CAR-NK in Different Forms

The efficacy of BCMA-CAR1 loaded with the NKG2D TM1 element, BCMA-CAR2 loaded with the NKG2D TM2 element, BCMA-CAR6 loaded with the NKG2D TM4 element, and BCMA-CAR4 and BCMA-CAR5 without NKG2D TM elements (see FIGS. 1 and 9 for the CAR structures; see Table 6 for the sequences) was assessed in an NCI H929-luc mouse model.

### 1. Preparation of BCMA CAR-NK Cells

Referring to the procedures in Example 4, BCMA CAR-NK cells were prepared using NK cells of different origins from that in Example 4. The NK cells were infected at a MOI=5, the amount of viruses was calculated according to the virus titer, and the viruses were added to a 24-well plate. The 24-well plate to which the viruses had been added was centrifuged at 2000 g at 4-8° C. for 60 min. The virus fluid supernatant was discarded. The NK cells were counted and added to the 24-well plate at 3E5 cells/well, and the mixture was centrifuged at 400 g at room temperature for 5 min. The 24-well plate was cultured in an incubator (37° C., 5% CO₂). On day 3, the transfected NK cells were transferred into a Non-Treated 6-well plate. On day 9, the CAR expression was detected, as shown in FIG. 10.

As can be seen from the results in FIG. 10, in terms of the expression rate of CAR in this study, the expression rate of CARs loaded with the NKG2D TM element in the NK cells was lower than those of the BCMA-CAR4 and BCMA-CAR5 in other forms; the expression rates of the BCMA-CAR1 and BCMA-CAR6 were higher than that of BCMA-CAR2.

2. Anti-Tumor Efficacy Assay of BCMA-BCMA CAR-NK Loaded with NKG2D-TM Element in NCI H929-Luc Mouse Tumor Model

To evaluate the anti-tumor efficacy of BCMA-BCMA CAR-NK loaded with the NKG2D-TM element, an anti-tumor efficacy study was conducted using a mouse myeloma venous graft tumor model. The procedures are as follows:

H929-Luc cells in the logarithmic growth phase and in good growth condition were collected, and grafted to NPG mice (combined immunodeficient mice) via the tail vein at 2×10⁶ cells/mouse. The body weight and the grafting condition of the mice were measured on the first day after the tumor grafting, and mice with a weight ranging from about 18.85-23.52 g were selected and randomized with an average weight of 21.92 g. On the first day (i.e., the day of grouping) after the tumor grafting, freshly prepared CAR-NK cells were injected into the mice via the tail vein (5×10⁶ cells/mouse) at an injection volume of 200 μL/mouse. The day of the CAR-NK cell injection was recorded as day 0. The mouse grouping and CAR-NK cell injection are shown in Table 10. The tumor growth fluorescence signal ROI value by an IVIS in vivo imaging system and the weight change were continuously monitored twice every week. The tumor growth inhibition was calculated and the calculation formula was as follows: Tumor growth inhibition TGI (%)=(photon signal value of mouse tumor in PBS group−photon signal value of mouse tumor in treatment group)/photon signal value of mouse tumor in PBS group×100%.

TABLE 10
Grouping of in vivo anti-tumor efficacy study

| | Number | | | | |
|---|---|---|---|---|---|
| | of | | | Route of | Frequency of |
| Group | animals | Therapeutic | Dose | administration | administration |
| G1 | 5 | Parental NK | 5 × 10⁶ | Tail vein | Once |
| | | | cells/mouse | injection | |
| G2 | 5 | BCMA-CAR 1 | 5 × 10⁶ | Tail vein | Once |
| | | | cells/mouse | injection | |
| G3 | 5 | BCMA- CAR 6 | 5 × 10⁶ | Tail vein | Once |
| | | | cells/mouse | injection | |
| G4 | 5 | BCMA-CAR 2 | 5 × 10⁶ | Tail vein | Once |
| | | | cells/mouse | injection | |
| G5 | 5 | BCMA-CAR 4 | 5 × 10⁶ | Tail vein | Once |
| | | | cells/mouse | injection | |
| G6 | 5 | BCMA-CAR 5 | 5 × 10⁶ | Tail vein | Once |
| | | | cells/mouse | injection | |

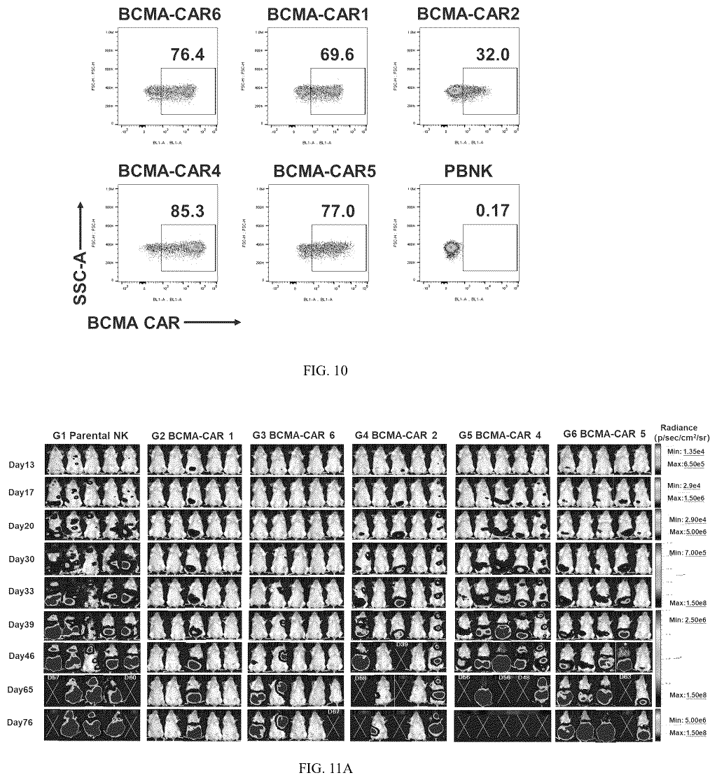

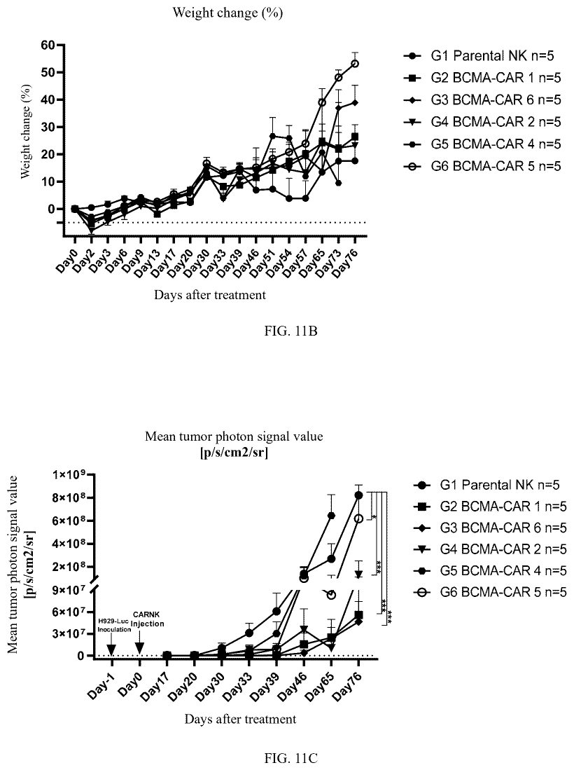

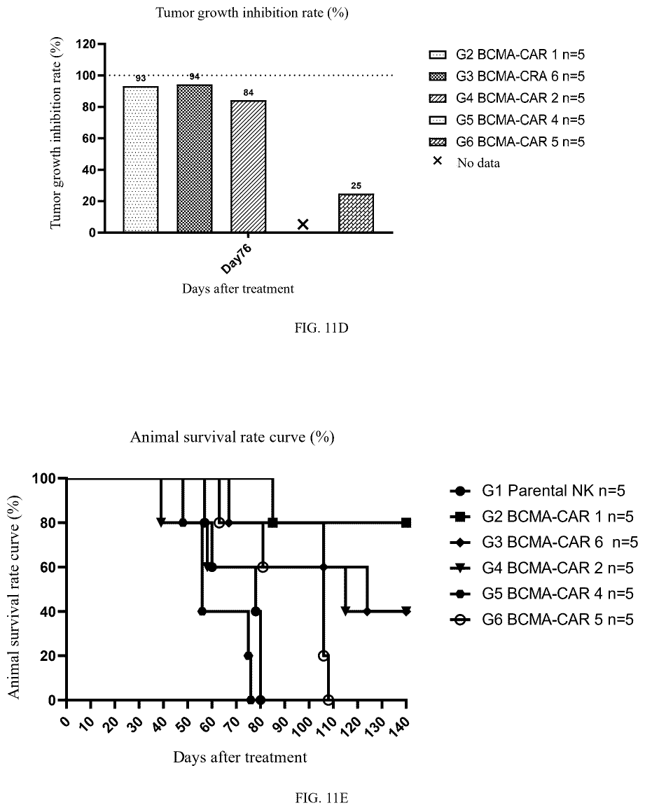

The detection results of the tumor growth fluorescence signal of the mice are shown in FIG. 11A. The results show that: On day 76 after the CAR-NK cell injection, in the BCMA-CAR1 treatment group, tumor growth volume in the mice was significantly inhibited, in which four animals were cured, and only one animal had recurrence of tumor growth; in the BCMA-CAR6 treatment group, tumor growth volume in the mice was significantly inhibited, but tumor progression in the animals of this group was not well controlled, in which two animals had tumor growth recurrence, tumors in two animals were completely regressed, and one animal died; in the BCMA-CAR2 treatment group, the rate of tumor growth in the mice was inhibited, in which one animal was cured, but four animals in this group had tumor recurrence, and two animals died; for BCMA-CAR4 and BCMA-CAR5, the tumor growth was inhibited on day 39 after the administration, and two groups exhibited poor tumor control effect with tumor recurrence later in the study; the mice in the BCMA-CAR4 group all died on day 76.

Meanwhile, the weight of the mice in the CAR-NK treatment groups fluctuated after the administration, but showed a generally upward increasing trend (as shown in FIG. 11B).

The photon values in mouse tumors were measured continuously after the administration, and except for the BCMA-CAR5 treatment group, the values of the remaining CAR NK treatment groups showed a decreasing trend as compared to the Parental NK group, with P<0.05* and P<0.001***. Particularly, the BCMA-CAR1 and BCMA-CAR6 treatment groups showed very significant therapeutic effects (as shown in FIG. 11C).

The tumor volume of the mice was measured on day 76, and the tumor growth inhibition was calculated. As shown in FIG. 11D, the tumor growth inhibition rates of the BCMA-CAR1, BCMA-CAR6, and BCMA-CAR4 treatment groups reached 93%, 94%, and 84%, respectively, suggesting good tumor growth inhibitory effects, while the tumor growth inhibition rate of the BCMA-CAR5 treatment group only reached 25%. The animals in the BCMA-CAR4 group all died before day 76, so the tumor inhibition rate of this group cannot be calculated.

The survival rate was continuously monitored during the study until day 130. As shown in FIG. 11E, in the parental NK treatment group, one animal died on day 57 and the mortality rate reached 100% on day 80; in the BCMA-CAR6 treatment group, one animal died on day 67 and the mortality rate was 60% by day 130; in the BCMA-CAR2 treatment group, one animal died on day 39 (after the detection of tumor growth fluorescence signal on the day 39, the mouse died on the same day) and the mortality rate was 60% by day 130; in the BCMA-CAR4 treatment group, one animal died on day 48 and the mortality rate reached 100% by day 76; in the BCMA-CAR5 treatment group, one animal died on day 63 and the mortality rate reached 100% by day 108; in the BCMA-CAR1 treatment group, only one animal died on day 85, showing a long-lasting therapeutic effect with better safety.

### Example 10. Preparation of GPRC5D Antibodies

10.1 Preparation of Anti-GPRC5D scFv

Anti-human GPRC5D monoclonal antibodies were produced by immunizing mice using conventional hybridoma methods. Chimeric antibodies containing the constant region of human IgG1 were constructed based on the sequences of the light and heavy chain variable regions obtained by sequencing, and monoclonal antibodies binding to both human and monkey GPRC5D-overexpressing cells, and NCI-H929 cells were screened and obtained by cell-based ELISA and FACS well known to those skilled in the art for subsequent humanization design.

By alignment with the IMGT database (http://imgt.cines.fr) for germline genes from heavy and light chain variable regions of human antibodies, humanized monoclonal antibodies were obtained by a conventional method for humanized antibodies in the art. The amino acid residues of the CDRs of the antibodies were identified and annotated by the Kabat numbering scheme. Subsequently, the cell-based ELISA was used for verification, and the final murine antibodies obtained were designated as GPRC5D-mab03 and GPRC5D-mab06, and the final humanized antibodies obtained were designated as GPRC5D-Hab03 and GPRC5D-Hab06. The sequences of the light and heavy chain variable regions of the antibodies are shown in Table 11, and the sequences of the CDRs are shown in Table 12.

TABLE 11
VL and VH sequences of anti-GPRC5D murine antibodies and humanized antibodies
thereof

| Antibody | Sequence No. | Amino acid sequence |
|---|---|---|
| GPRC5D-mab03-VL | SEQ ID NO: 77 | DIVMTQSQKFMSTTVGDRVTITCKASQNVGTAVAWYQQKPGQ |
| | | SPKLLISSASNRYTGVPERFTGSGSGTDFTLTIRNMQSEDLADYF |
| | | CQQYSSYPWTFGGGTELEIK |
| GPRC5D-mab03-VH | SEQ ID NO: 78 | QAYLQQSGAELVRPGASVKMSCKASGYTFTSYNMNWVKQTP |
| | | RQGLEWIGAIYPGNGDTSYNQKFKGKATLTVDKSSSTAYMQLS |
| | | SLTSEDSAVYFCARVRLRYAMDYWGQGTSVTVSS |
| GPRC5D-Hab03.VL1 | SEQ ID NO: 79 | DIQMTQSPSSLSASVGDRVTITCKASQNVGTAVAWYQQKPGKS |
| | | PKLLISSASNRYTGVPSRFSGSGSGTDFTLTISSLQPEDFATYYCQ |
| | | QYSSYPWTFGGGTKVEIK |
| GPRC5D-Hab03.VH4a | SEQ ID NO: 80 | EAYLQQSGAEVKKPGASVKVSCKASGYTFTSYNMNWVRQAP |
| | | GQRLEWMGAIYPGNADTSYNQKFKGRVTITVDKSASTAYMEL |
| | | SSLRSEDTAVYYCARVRLRYAMDYWGQGTTVTVSS |
| GPRC5D-mab06-VL | SEQ ID NO: 81 | DIVMTQSHKFMSTSVGDRVSITCKASQDVRTAVAWYQQKPGQS |
| | | PKLLIYWASTRHTGVPDRFTGSRSGTDYTLTISSVQAEDLALYY |
| | | CQQHYSTPLTFGAGTKLELK |
| GPRC5D-mab06-VH | SEQ ID NO: 131 | QVQLKQSGPGLVQPSQSLSITCTVSGFSLTSYGVNWIRQSPGKG |
| | | LEWLGVIWSGGNTDYNAAFRSRLSISKDNSKSQVFFKMNSLQ |
| | | ADDTAIYYCARGQLGRPYWYFDVWGTGTSVTVSS |
| GPRC5D-Hab06.VL4 | SEQ ID NO: 82 | DIQMTQSPSSLSASVGDRVTITCKASQDVRTAVAWYQQKPGKS |
| | | PKLLIYWASTRHTGVPDRFSGSGSGTDFTLTISSLQPEDVATYYC |
| | | QQHYSTPLTFGQGTKLEIK |
| GPRC5D-Hab06.VH4 | SEQ ID NO: 83 | QVQLKESGPVLVKPTETLTLTCTVSGFSLTSYGVNWIRQPPGKA |
| | | LEWLAVIWSGGNTDYNAAFRSRLTISKDNSKSQVVLTMTNMD |
| | | PVDTATYYCARGQLGRPYWYFDVWGQGTTVTVSS |

TABLE 12
Sequences of Kabat-numbered LCDRs and HCDRs of anti-GPRC5D antibodies

| Variable region | CDR1 | CDR2 | CDR3 |
|---|---|---|---|
| GPRC5D-mab03-VL | KASQNVGTAVA | SASNRYT | QQYSSYPWT |
| GPRC5D-Hab03.VL1 | (SEQ ID NO: 84) | (SEQ ID NO: 85) | (SEQ ID NO: 86) |
| GPRC5D-mab03-VH | SYNMN | AIYPGNGDTSYNQKFKG | VRLRYAMDY |
| | (SEQ ID NO: 87) | (SEQ ID NO: 88) | (SEQ ID NO: 89) |
| GPRC5D-Hab03.VH4a | SYNMN | AIYPGNADTSYNQKFKG | VRLRYAMDY |
| | (SEQ ID NO: 90) | (SEQ ID NO: 91) | (SEQ ID NO: 92) |
| GPRC5D-mab06-VL | KASQDVRTAVA | WASTRHT | QQHYSTPLT |
| GPRC5D-Hab06.VL4 | (SEQ ID NO: 93) | (SEQ ID NO: 94) | (SEQ ID NO: 95) |
| GPRC5D-mab06-VH | SYGVN | VIWSGGNTDYNAAFRS | GQLGRPYWYFDV |
| GPRC5D-Hab06.VH4 | (SEQ ID NO: 96) | (SEQ ID NO: 97) | (SEQ ID NO: 98) |

### 10.2 Preparation of Anti-GPRC5D Nanobodies

Vicugna pacos-derived VHH antibodies were prepared using conventional VHH antibody screening methods in the art, and the immunogen used was the human GPRC5D protein. The amino acid residues of the CDRs of the antibodies were identified and annotated by the IMGT or Kabat numbering scheme. Finally, two Vicugna pacos sequences were obtained and designated as GPRC5D-Lab03 and GPRC5D-Lab04; two humanized sequences were obtained and designated as GPRC5D-Hab03-H10 and GPRC5D-Hab04-H5. The amino acid sequences are shown in Table 13, and the sequences of the numbered CDRs are shown in Table 14.

Camelid-derived VHH antibodies were prepared using conventional VHH antibody screening methods in the art, and the immunogen used was the human GPRC5D protein. The antibodies were annotated by IMGT or Kabat. After FACS verification, a plurality of camelid antibodies with relatively high affinity and humanized antibodies thereof were obtained. The camelid antibodies were designated as GPRC5D-Lab05 and GPRC5D-Lab06, and the humanized antibodies thereof were designated as GPRC5D-Hab05-H2 and GPRC5D-Hab06-H1. The sequences of the nanobodies are shown in Table 13, and the sequences of the CDRs are shown in Table 14.

TABLE 13
Amino acid sequences of anti-GPRC5D nanobodies

| Antibody | Sequence No. | Amino acid sequence |
|---|---|---|
| GPRC5D-Lab03 | SEQ ID NO: 99 | DVQLQASGGGSVQAGGSLRLSCAASGDTANCIGWFRQAPGKGR |
| | | EGVAAIYTGLGNTYYANSAKGRFTIAQDNAKNTVYLQMNSLKP |
| | | EDSAMYYCAAGGILGRFCSRYFGQGTQVTVSS |
| GPRC5D-Lab04 | SEQ ID NO: 100 | EVQLQASGGGSVQAGGSLRLSCVASGFPYSTSRVGWFRQAPGK |
| | | EREGVATIVPGTGYTYYADSVKGRFTISQDSAKNTVYLQMNSLK |
| | | PEDTAVYYCAAGDRRLLLNLLAPADYFYWGQGTQVTVSS |
| GPRC5D-Hab03-H10 | SEQ ID NO: 101 | EVQLVESGGGLVQPGGSLRLSCAASGDTANCIGWFRQAPGKGR |
| | | EGVAAIYTGLGNTYYANSAKGRFTISRDNAKNSVYLQMNSLRA |
| | | EDTAVYYCAAGGILGRFCSRYFGQGTMVTVSS |
| GPRC5D-Hab04-H5 | SEQ ID NO: 102 | EVQLVESGGGLVQPGGSLRLSCAASGFPYSTSRVGWFRQAPGKE |
| | | REGVATIVPGTGYTYYVDSVKGRFTISQDNAKNSVYLQMNSLRA |
| | | EDTAVYYCAAGDRRLLLNLLAPADYFYWGQGTMVTVSS |
| GPRC5D-Lab05 | SEQ ID NO: 103 | QVQLVESGGGLVQPGGSLRLSCAASGILVSAYVMAWYRQPPGK |
| | | QRDLVARLSTDGRTTYADSVKGRFTISKDNAKSTLYLQMNTLKS |
| | | EDTAVYYCYAERSRGHWGQGTQVTVSS |
| GPRC5D-Lab06 | SEQ ID NO: 104 | QVQLVESGGGLVQPGGSLRLSCTASGFTFSSAWMYWFRQAPGK |
| | | GLEWVSHIDPRGGSTYYADSVKGRFTISRDNAKNTLYLQMRSLK |
| | | SEDTAVYYCAKDLRGLGRGQGTQVTVSS |
| GPRC5D-Hab05-H2 | SEQ ID NO: 105 | EVQLVESGGGLVQPGGSLRLSCAASGILVSAYVMAWYRQAPGK |
| | | QRVLVSRLSTDGRTTYADSVKGRFTISKDNAKNTLYLQMNSLRA |
| | | EDTAVYYCYAERSRGHWGQGTMVTVSS |
| GPRC5D-Hab06-H1 | SEQ ID NO: 106 | EVQLVESGGGLVQPGGSLRLSCAASGFTFSSAWMYWFRQAPGK |
| | | GLEWVSHIDPRGGSTYYADSVKGRFTISRDNSKNTLYLQMNSLR |
| | | AEDTAVYYCAKDLRGLGRGQGTMVTVSS |

TABLE 14
Sequences of CDRs of anti-GPRC5D nanobodies

| | Numbering | | | |
|---|---|---|---|---|
| Variable region | scheme | CDR1 | CDR2 | CDR3 |
| GPRC5D-Lab03 | Kabat | CIG | AIYTGLGNTYYANSAKG | GGILGRFCSRY |
| GPRC5D-Hab03-H10 | | (SEQ ID NO: 107) | (SEQ ID NO: 108) | (SEQ ID NO: 109) |
| GPRC5D-Lab04 | IMGT | GFPYSTSR | IVPGTGYT | AAGDRRLLLNLLA |
| GPRC5D-Hab04-H5 | | (SEQ ID NO: 110) | (SEQ ID NO: 111) | PADYFY |
| | | | | (SEQ ID NO: 112) |
| GPRC5D-Lab05 | IMGT | GILVSAYV | LSTDGRT | YAERSRGH |
| GPRC5D-Hab05-H2 | | (SEQ ID NO: 113) | (SEQ ID NO: 114) | (SEQ ID NO: 115) |
| GPRC5D-Lab06 | Kabat | SAWMY | HIDPRGGSTYYADSVKG | DLRGLG |
| GPRC5D-Hab06-H1 | | (SEQ ID NO: 116) | (SEQ ID NO: 117) | (SEQ ID NO: 118) |

### Example 11. Construction of 4-1BB Structure Chimeric Antigen Receptors and Screening of Extracellular Antibody Combinations

### 11.1 Construction of 4-1BB Structure CAR-NK

By using the BCMA-VHH antibodies screened and obtained in Example 1, i.e., the hu-VHH2 and GPRC5D-scFv antibodies, dual-target chimeric antigen receptors (BI-CARs) targeting BCMA and GPRC5D were designed and constructed as shown in FIG. 12. The nucleic acid sequences of the BI-CARs were loaded into a retroviral vector using the biological methods in Examples 3 and 4 to construct target plasmids and viral vectors. The NK cells were transfected to prepare CAR-NK cells. The structures of BI-CARs 01-04 are shown in FIGS. 12A-12D, respectively, and the structure of BI-CAR05 is shown in FIG. 12A. The amino acid sequences of the domains of the BI-CARs and exemplary BI-CARs 01-05 sequences are shown in Table 15.

TABLE 15
Domains of BI-CAR and exemplary BI-CAR amino acid sequences

| Sequence | Sequence No. | Amino acid sequence |
|---|---|---|
| CD8α signal | SEQ ID NO: 43 | MALPVTALLLPLALLLHAARP |
| peptide | | |
| CD8 hinge region | SEQ ID NO: 45 | TTTPAPRPPTPAPTIASQPLSLRPEACRPAAGGAVHTRGLDFACD |
| CD8 | SEQ ID NO: | IYIWAPLAGTCGVLLLSLVITLYC |
| transmembrane | 119 | |
| region | | |
| CD137 | SEQ ID NO: | KRGRKKLLYIFKQPFMRPVQTTQEEDGCSCRFPEEEEGGCEL |
| co-stimulatory | 120 | |
| domain | | |
| CD3ζ | SEQ ID NO: 55 | RVKFSRSADAPAYQQGQNQLYNELNLGRREEYDVLDKRRGRDPEMGG |
| | | KPRRKNPQEGLYNELQKDKMAEAYSEIGMKGERRRGKGHDGLYQGLS |
| | | TATKDTYDALHMQALPPR |
| Self-cleaving | SEQ ID NO: 56 | ATNFSLLKQAGDVEENPGP |
| peptide P2A | | |
| IL15 | SEQ ID NO: 57 | MRISKPHLRSISIQCYLCLLLNSHFLTEAGIHVFILGCFSAGLPKTEANWV |
| | | NVISDLKKIEDLIQSMHIDATLYTESDVHPSCKVTAMKCFLLELQVISLES |
| | | GDASIHDTVENLIILANNSLSSNGNVTESGCKECEELEEKNIKEFLQSFV |
| | | HIVQMFINTS |
| BI-CAR01 | SEQ ID NO: | MALPVTALLLPLALLLHAARPDIQMTQSPSSLSASVGDRVTITCKASQD |
| | 121 | VRTAVAWYQQKPGKSPKLLIYWASTRHTGVPDRFSGSGSGTDFTLTISSL |
| | | QPEDVATYYCQQHYSTPLTFGQGTKLEIKGGGGSGGGGSGGGGSQVQL |
| | | KESGPVLVKPTETLTLTCTVSGFSLTSYGVNWIRQPPGKALEWLAVIWS |
| | | GGNTDYNAAFRSRLTISKDNSKSQVVLTMTNMDPVDTATYYCARGQLG |
| | | RPYWYFDVWGQGTTVTVSSGGGGSGGGGSGGGGSggggsEVQLVESGG |
| | | GLVQPGGSLRLSCAASESISSIHIMAWYRQAPGKQRELVAGIRNDGSTVY |
| | | VDSVKGRFTISRDNAKNSVYLQMNSLRAEDTAVYYCNADQGFGSYSE |
| | | WERRSRWGQGTTVTVSSTTTPAPRPPTPAPTIASQPLSLRPEACRPAAGG |
| | | AVHTRGLDFACDIYIWAPLAGTCGVLLLSLVITLYCKRGRKKLLYIFKQP |
| | | FMRPVQTTQEEDGCSCRFPEEEEGGCELRVKFSRSADAPAYQQGQNQL |
| | | YNELNLGRREEYDVLDKRRGRDPEMGGKPRRKNPQEGLYNELQKDK |
| | | MAEAYSEIGMKGERRRGKGHDGLYQGLSTATKDTYDALHMQALPPRG |
| | | SGATNFSLLKQAGDVEENPGPMRISKPHLRSISIQCYLCLLLNSHFLTEA |
| | | GIHVFILGCFSAGLPKTEANWVNVISDLKKIEDLIQSMHIDATLYTESDV |
| | | HPSCKVTAMKCFLLELQVISLESGDASIHDTVENLIILANNSLSSNGNVT |
| | | ESGCKECEELEEKNIKEFLQSFVHIVQMFINTS |
| BI-CAR02 | SEQ ID NO: | MALPVTALLLPLALLLHAARPEVQLVESGGGLVQPGGSLRLSCAASESIS |
| | 122 | SIHIMAWYRQAPGKQRELVAGIRNDGSTVYVDSVKGRFTISRDNAKNSV |
| | | YLQMNSLRAEDTAVYYCNADQGFGSYSEWERRSRWGQGTTVTVSSGG |
| | | GGSGGGGSGGGGSggggsDIQMTQSPSSLSASVGDRVTITCKASQDVRTA |
| | | VAWYQQKPGKSPKLLIYWASTRHTGVPDRFSGSGSGTDFTLTISSLQPED |
| | | VATYYCQQHYSTPLTFGQGTKLEIKGGGGSGGGGSGGGGSQVQLKESG |
| | | PVLVKPTETLTLTCTVSGFSLTSYGVNWIRQPPGKALEWLAVIWSGGNT |
| | | DYNAAFRSRLTISKDNSKSQVVLTMTNMDPVDTATYYCARGQLGRPY |
| | | WYFDVWGQGTTVTVSSTTTPAPRPPTPAPTIASQPLSLRPEACRPAAGGA |
| | | VHTRGLDFACDIYIWAPLAGTCGVLLLSLVITLYCKRGRKKLLYIFKQPF |
| | | MRPVQTTQEEDGCSCRFPEEEEGGCELRVKFSRSADAPAYQQGQNQLY |
| | | NELNLGRREEYDVLDKRRGRDPEMGGKPRRKNPQEGLYNELQKDKM |
| | | AEAYSEIGMKGERRRGKGHDGLYQGLSTATKDTYDALHMQALPPRGS |
| | | GATNFSLLKQAGDVEENPGPMRISKPHLRSISIQCYLCLLLNSHFLTEAGI |
| | | HVFILGCFSAGLPKTEANWVNVISDLKKIEDLIQSMHIDATLYTESDVHP |
| | | SCKVTAMKCFLLELQVISLESGDASIHDTVENLIILANNSLSSNGNVTES |
| | | GCKECEELEEKNIKEFLQSFVHIVQMFINTS |
| BI-CAR03 | SEQ ID NO: | MALPVTALLLPLALLLHAARPDIQMTQSPSSLSASVGDRVTITCKASQD |
| | 123 | VRTAVAWYQQKPGKSPKLLIYWASTRHTGVPDRFSGSGSGTDFTLTISSL |
| | | QPEDVATYYCQQHYSTPLTFGQGTKLEIKGGGGSGGGGSGGGGSQVQL |
| | | KESGPVLVKPTETLTLTCTVSGFSLTSYGVNWIRQPPGKALEWLAVIWS |
| | | GGNTDYNAAFRSRLTISKDNSKSQVVLTMTNMDPVDTATYYCARGQLG |
| | | RPYWYFDVWGQGTTVTVSSTTTPAPRPPTPAPTIASQPLSLRPEACRPAA |
| | | GGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYQQGQN |
| | | QLYNELNLGRREEYDVLDKRRGRDPEMGGKPRRKNPQEGLYNELQKD |
| | | KMAEAYSEIGMKGERRRGKGHDGLYQGLSTATKDTYDALHMQALPPR |
| | | LEGGGEGRGSLLTCGDVEENPGPRMALPVTALLLPLALLLHAARPEVQL |
| | | VESGGGLVQPGGSLRLSCAASESISSIHIMAWYRQAPGKQRELVAGIRND |
| | | GSTVYVDSVKGRFTISRDNAKNSVYLQMNSLRAEDTAVYYCNADQGF |
| | | GSYSEWERRSRWGQGTTVTVSSTTTPAPRPPTPAPTIASQPLSLRPEACR |
| | | PAAGGAVHTRGLDFACDIYIWAPLAGTCGVLLLSLVITLYCKRGRKKLL |
| | | YIFKQPFMRPVQTTQEEDGCSCRFPEEEEGGCELRVKFSRSADAPAYQQ |
| | | GQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQA |
| | | LPPRGSGATNFSLLKQAGDVEENPGPMRISKPHLRSISIQCYLCLLLNSH |
| | | FLTEAGIHVFILGCFSAGLPKTEANWVNVISDLKKIEDLIQSMHIDATLYT |
| | | ESDVHPSCKVTAMKCFLLELQVISLESGDASIHDTVENLIILANNSLSSN |
| | | GNVTESGCKECEELEEKNIKEFLQSFVHIVQMFINTS |
| BI-CAR04 | SEQ ID NO: | MALPVTALLLPLALLLHAARPEVQLVESGGGLVQPGGSLRLSCAASESIS |
| | 124 | SIHIMAWYRQAPGKQRELVAGIRNDGSTVYVDSVKGRFTISRDNAKNSV |
| | | YLQMNSLRAEDTAVYYCNADQGFGSYSEWERRSRWGQGTTVTVSSTT |
| | | TPAPRPPTPAPTIASQPLSLRPEACRPAAGGAVHTRGLDFACDIYIWAPLA |
| | | GTCGVLLLSLVITLYCKRGRKKLLYIFKQPFMRPVQTTQEEDGCSCRFPE |
| | | EEEGGCELRVKFSRSADAPAYQQGQNQLYNELNLGRREEYDVLDKRRG |
| | | RDPEMGGKPRRKNPQEGLYNELQKDKMAEAYSEIGMKGERRRGKGHD |
| | | GLYQGLSTATKDTYDALHMQALPPRLEGGGEGRGSLLTCGDVEENPGP |
| | | RMALPVTALLLPLALLLHAARPDIQMTQSPSSLSASVGDRVTITCKASQ |
| | | DVRTAVAWYQQKPGKSPKLLIYWASTRHTGVPDRFSGSGSGTDFTLTISS |
| | | LQPEDVATYYCQQHYSTPLTFGQGTKLEIKGGGGSGGGGSGGGGSQVQ |
| | | LKESGPVLVKPTETLTLTCTVSGFSLTSYGVNWIRQPPGKALEWLAVIWS |
| | | GGNTDYNAAFRSRLTISKDNSKSQVVLTMTNMDPVDTATYYCARGQLG |
| | | RPYWYFDVWGQGTTVTVSSTTTPAPRPPTPAPTIASQPLSLRPEACRPAA |
| | | GGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYQQGQN |
| | | QLYNELNLGRREEYDVLDKRRGRDPEMGGKPRRKNPQEGLYNELQKD |
| | | KMAEAYSEIGMKGERRRGKGHDGLYQGLSTATKDTYDALHMQALPPR |
| | | GSGATNFSLLKQAGDVEENPGPMRISKPHLRSISIQCYLCLLLNSHFLTE |
| | | AGIHVFILGCFSAGLPKTEANWVNVISDLKKIEDLIQSMHIDATLYTESD |
| | | VHPSCKVTAMKCFLLELQVISLESGDASIHDTVENLIILANNSLSSNGNV |
| | | TESGCKECEELEEKNIKEFLQSFVHIVQMFINTS |
| BI-CAR05 | SEQ ID NO: | MALPVTALLLPLALLLHAARPDIQMTQSPSSLSASVGDRVTITCKASQN |
| | 125 | VGTAVAWYQQKPGKSPKLLISSASNRYTGVPSRFSGSGSGTDFTLTISSL |
| | | QPEDFATYYCQQYSSYPWTFGGGTKVEIKGGGGSGGGGSGGGGSEAYL |
| | | QQSGAEVKKPGASVKVSCKASGYTFTSYNMNWVRQAPGQRLEWMGA |
| | | IYPGNADTSYNQKFKGRVTITVDKSASTAYMELSSLRSEDTAVYYCARV |
| | | RLRYAMDYWGQGTTVTVSSGGGGSGGGGSGGGGSggggsEVQLVESGG |
| | | GLVQPGGSLRLSCAASESISSIHIMAWYRQAPGKQRELVAGIRNDGSTVY |
| | | VDSVKGRFTISRDNAKNSVYLQMNSLRAEDTAVYYCNADQGFGSYSE |
| | | WERRSRWGQGTTVTVSSTTTPAPRPPTPAPTIASQPLSLRPEACRPAAGG |
| | | AVHTRGLDFACDIYIWAPLAGTCGVLLLSLVITLYCKRGRKKLLYIFKQP |
| | | FMRPVQTTQEEDGCSCRFPEEEEGGCELRVKFSRSADAPAYQQGQNQL |
| | | YNELNLGRREEYDVLDKRRGRDPEMGGKPRRKNPQEGLYNELQKDK |
| | | MAEAYSEIGMKGERRRGKGHDGLYQGLSTATKDTYDALHMQALPPRG |
| | | SGATNFSLLKQAGDVEENPGPMRISKPHLRSISIQCYLCLLLNSHFLTEA |
| | | GIHVFILGCFSAGLPKTEANWVNVISDLKKIEDLIQSMHIDATLYTESDV |
| | | HPSCKVTAMKCFLLELQVISLESGDASIHDTVENLIILANNSLSSNGNVT |
| | | ESGCKECEELEEKNIKEFLQSFVHIVQMFINTS |

### 11.2 Determination of Expression Rate of Chimeric Antigen Receptors

On day 6 after the retroviral infection of NK cells, the expression rate of CARs on the surface of NK cells was detected by flow cytometry. The molecule expression on the surface of the BCMA-CAR NK cells and GPRC5D-CAR NK cells of the BI-CARs was detected referring to the method for detecting the CAR expression rate in the NK cells in Example 5.1.

The detection results of the expression of the BI-CARs by FACS are shown in Table 16. The results show that for the forms as shown in FIGS. 12A and 12B, the expression of anti-BCMA and anti-GPRC5D was generally higher with no position-dependence; however, for the forms shown in FIGS. 12C and 12D, the expression of BCMA-CAR or GPRC5D-CAR was affected by their positions.

TABLE 16
Detection of BI-CAR expression rate

| | BCMA-CAR | GPRC5D-CAR |
|---|---|---|
| BI-CAR | expression | expression |
| BI-CAR01 | 85% | 66% |
| BI-CAR02 | 84% | 64% |
| BI-CAR03 | 45% | 59% |
| BI-CAR04 | 73% | 20% |
| BI-CAR05 | 69% | 46% |
| NK | 1% | 0% |

### 11.3 Detection of 4 h In Vitro Killing Rate of BI-CAR-NK on Target Cells

On day 6 after the retroviral infection, the NK cells were subjected to a 4 h in vitro killing assay: The target cells were NCI H929, RPMI 8226, MOLP8, and NCI H929-hBCMA-KO, all of which were transfected with the luciferase reporter gene by conventional gene manipulation methods, wherein the NCI H929-hBCMA-KO cells were NCI H929 cells with BCMA gene knockout using conventional gene manipulation methods. The target cells diluted with a 1640 culture medium were added to a white opaque 96-well plate at 2×10⁴ cells/50 μL/well, and the NK cells were added to the target cells described above in effector-to-target ratios of 5:1, 2.5:1, and 1.25:1. The killing rate was measured and calculated by referring to the method in Example 6.

The 4 h in vitro cell killing effects of the BI-CARs on the target cells described above are detailed in Table 17. In an effector-to-target ratio of 5:1, BI-CAR01 to BI-CAR05 had specific killing effects on NCI H929-Lu and NCI H929-hBCMA-KO-Lu. BI-CAR01 to BI-CAR03 and BI-CAR05 generally had good specific killing effects on four tumor cells, of which BI-CAR01, BI-CAR-03, and BI-CAR05 had higher killing effects on NCI H929-hBCMA-KO-Lu tumor cells, indicating that BI-CAR01, BI-CAR03, and BI-CAR05 forms have good killing functionality against tumor cells with down-regulated or deleted BCMA expression.

TABLE 17
Results of 4 h in vitro rapid killing rate of BI-CAR-NK on target cells

| | | | | | | | | | Killing |
|---|---|---|---|---|---|---|---|---|---|
| | | | | | | | Killing efficiency (%) | | efficiency (%) |
| | Killing efficiency (%) | | | Killing efficiency (%) | | | (target cell: NCI | | (target cell: |
| | (target cell: NCI H929-Lu) | | | (target cell: RPMI 8226-Lu) | | | H929-hBCMA-KO-Lu) | | MOLP8-Lu) |
| BI-CAR | 5:1 | 2.5:1 | 1.25:1 | 5:1 | 2.5:1 | 1.25:1 | 5:1 | 2.5:1 | 1.25:1 |
| BI-CAR01 | 91 | 33 | 9 | 77 | 38 | — | 81 | 34 | 70 |
| BI-CAR02 | 94 | 61 | 27 | 80 | 45 | 15 | 55 | — | 62 |
| BI-CAR03 | 94 | 65 | 41 | 69 | 58 | 22 | 65 | 23 | 72 |
| BI-CAR04 | 71 | — | — | 45 | — | — | 27 | — | 54 |
| BI-CAR05 | 74.38 | 2.05 | — | 50.86 | 1.81 | — | 69.12 | 28.00 | 68.59 |
| NK | — | — | — | 42 | 23 | 12 | — | — | 31 |

Note:
“—” denotes that no killing effect was detected.

### Example 12. Design of NKG2D-F Structure Chimeric Antigen Receptors

### 12.1 Construction of NKG2D-F Structure Chimeric Antigen Receptors.

To verify the effect of different transmembrane domains on the activity of the chimeric antigen receptor, a dual-target chimeric antigen receptor (designated as BI-CAR06) targeting BCMA and GPRC5D having the structure shown in FIG. 13 was designed and constructed by using the antibodies BCMA-Hu-VHH2 and GPRC5D-Hab06 corresponding to BI-CAR01 and BI-CAR03 screened and obtained in Example 11. The dual-target chimeric antigen receptor targeting BCMA and GPRC5D includes CD8a signal peptide (SP), anti-GPRC5D-scFv and anti-BCMA-VHH, CD8 hinge region, NKG2D transmembrane region, 2B4 co-stimulatory domain and CD35, and IL15 linked by self-cleaving peptide P2A. The specific sequences are detailed in Table 18. The nucleic acid sequences were loaded into a retroviral vector to construct target plasmids by referring to the molecular biological method in Examples 3 and 4.

TABLE 18
Domains of BI-CAR and exemplary BI-CAR amino acid sequences

| Sequence | Sequence No. | Amino acid sequence |
|---|---|---|
| NKG2D | SEQ ID NO: 48 | SPFFFCCFIAVAMGIRFIIMVAIWSAVFLNS |
| transmembrane region | | |
| (NKG2D-TM1) | | |
| 2B4 co-stimulatory | SEQ ID NO: 53 | WRRKRKEKQSETSPKEFLTIYEDVKDLKTRRNHEQEQTFPGGGS |
| domain | | TIYSMIQSQSSAPTSQEPAYTLYSLIQPSRKSGSRKRNHSPSFNSTI |
| (2B4 intracellular | | YEVIGKSQPKAQNPARLSRKELENFDVYS |
| region) | | |
| BI-CAR06 | SEQ ID NO: 126 | MALPVTALLLPLALLLHAARPDIQMTQSPSSLSASVGDRVTITCK |
| | | ASQDVRTAVAWYQQKPGKSPKLLIYWASTRHTGVPDRFSGSGSG |
| | | TDFTLTISSLQPEDVATYYCQQHYSTPLTFGQGTKLEIKGGGGSGG |
| | | GGSGGGGSQVQLKESGPVLVKPTETLTLTCTVSGFSLTSYGVNWI |
| | | RQPPGKALEWLAVIWSGGNTDYNAAFRSRLTISKDNSKSQVVLT |
| | | MTNMDPVDTATYYCARGQLGRPYWYFDVWGQGTTVTVSSGGG |
| | | GSGGGGSGGGGSGGGGSEVQLVESGGGLVQPGGSLRLSCAASES |
| | | ISSIHIMAWYRQAPGKQRELVAGIRNDGSTVYVDSVKGRFTISRD |
| | | NAKNSVYLQMNSLRAEDTAVYYCNADQGFGSYSEWERRSRWG |
| | | QGTTVTVSSTTTPAPRPPTPAPTIASQPLSLRPEACRPAAGGAVHT |
| | | RGLDFACDSPFFFCCFIAVAMGIRFIIMVAIWSAVFLNSWRRKRKE |
| | | KQSETSPKEFLTIYEDVKDLKTRRNHEQEQTFPGGGSTIYSMIQSQ |
| | | SSAPTSQEPAYTLYSLIQPSRKSGSRKRNHSPSFNSTIYEVIGKSQP |
| | | KAQNPARLSRKELENFDVYSRVKFSRSADAPAYQQGQNQLYNEL |
| | | NLGRREEYDVLDKRRGRDPEMGGKPRRKNPQEGLYNELQKDK |
| | | MAEAYSEIGMKGERRRGKGHDGLYQGLSTATKDTYDALHMQAL |
| | | PPRGSGATNFSLLKQAGDVEENPGPMRISKPHLRSISIQCYLCLLL |
| | | NSHFLTEAGIHVFILGCFSAGLPKTEANWVNVISDLKKIEDLIQSM |
| | | HIDATLYTESDVHPSCKVTAMKCFLLELQVISLESGDASIHDTVEN |
| | | LIILANNSLSSNGNVTESGCKECEELEEKNIKEFLQSFVHIVQMFI |
| | | NTS |

12.2 Determination of CAR Expression Rate of CAR-NK with Different Structures

Referring to the methods shown in Examples 3 and 4, BI-CAR-NK cells with different structures of transmembrane regions and intracellular stimulatory domains were prepared by transfecting NK cells at the same MOI, and the CAR expression was detected by referring to the method in Example 5.1. The results are shown in Table 19. The results suggest that the CAR expression shown in FIG. 12A was superior to the CAR expression shown in FIG. 12C.

TABLE 19
Results of CAR expression rate of
CAR-NK with different structures

| | BCMA-CAR | GPRC5D-CAR |
|---|---|---|
| BI-CAR | expression | expression |
| BI-CAR05 | 72% | 51% |
| BI-CAR01 | 85% | 65% |
| BI-CAR06 | 55% | 21% |
| BI-CAR03 | 45% | 36% |
| NK | 2% | 2% |

12.3 Determination of Proliferation Rate of CAR-NK Cells with Different Structures

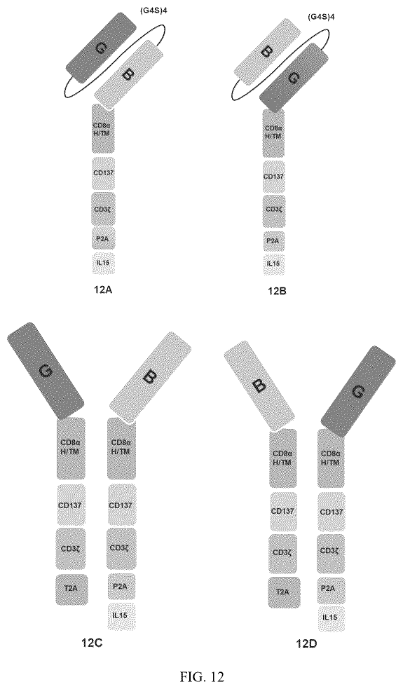

The proliferation of BI-CAR cells was detected by AO/PI counting, and the results are shown in FIG. 14. On day 14 after the NK activation, the descending proliferation rate order of BI-CAR cells was BI-CAR06 (5566 folds)>NK (3124 folds)>BI-CAR03 (1705 folds)>BI-CAR05 (1245.2 folds)>BI-CAR01 (1012 folds). As can be seen from the results described above, the proliferation rates of the optimized BI-CAR cells loaded with the NKG2D TM element were significantly superior to and higher than those of the BI-CAR cells using other transmembrane elements. 12.4 Determination of 4 h killing rate of CAR-NK cells with different structures

On day 6 after the retroviral infection, NK cells were subjected to a 4 h in vitro killing assay: The target cells NCI H929, RPMI 8226, MOLP8, and NCI H929-hBCMA-KO diluted with a 1640 culture medium were added to a white opaque 96-well plate at 2×10⁴ cells/50 μL/well, and the NK cells were added to the target cells described above in effector-to-target ratios of 9:1 and 3:1. The killing rate was measured and calculated by referring to the method in Example 6.

The 4 h in vitro cell killing effects of the BI-CARs on the target cells described above are detailed in Table 20. The results show that BI-CAR05, BI-CAR01, BI-CAR-03, and BI-CAR-06 all exhibited strong tumor cell killing functions.

TABLE 20
Results of 4 h rapid killing rate of CAR-NK cells with different structures

| | Killing efficiency | | Killing efficiency (%) | | Killing efficiency (%) | | Killing efficiency (%) | |
|---|---|---|---|---|---|---|---|---|
| | (%) (target cell: NCI | | (target cell: RPMI | | (target cell: | | (target cell: NCI | |
| | H929-Lu) | | 8226-Lu) | | MOLP8-Lu) | | H929-hBCMA-KO-Lu) | |
| BI-CAR | 9:1 | 3:1 | 9:1 | 3:1 | 9:1 | 3:1 | 9:1 | 3:1 |
| BI-CAR05 | 93.49 | 74.73 | 81.96 | 53.10 | 86.81 | 72.82 | 90.59 | 65.48 |
| BI-CAR01 | 96.02 | 69.67 | 80.20 | 39.98 | 89.48 | 62.93 | 94.25 | 50.57 |
| BI-CAR06 | 87.27 | 36.89 | 62.91 | 8.76 | 65.01 | 35.57 | 78.49 | 16.77 |
| BI-CAR03 | 89.85 | 52.22 | 67.07 | 29.89 | 83.97 | 51.74 | 89.32 | 56.34 |
| NK | — | — | — | 9.50 | 16.30 | −0.50 | — | — |

Note:
“—” denotes that no killing effect was detected.

12.5 Detection of Multiple-Run Killing Effects of CAR-NK with Different Structures

On day 6 after the retroviral infection of NK cells, the multiple-run in vitro killing effects of BI-CAR01, BI-CAR-03, BI-CAR-05, and BI-CAR-06 on RPMI 8226 cells and MOLP8 cells were detected. The multiple-run in vitro killing effects on RPMI 8226 cells and MOLP8 cells were determined by referring to the method in Example 7.

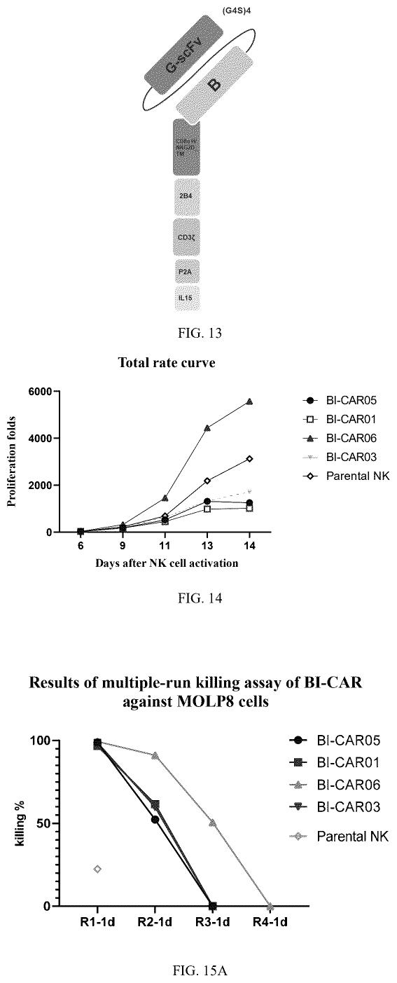

The results for the multiple-run killing assay of BI-CAR NK cells are shown in FIG. 15. As shown in FIG. 15, the killing effect of BI-CAR06 loaded with the NKG2D TM element was superior to those of the BI-CARs in other forms; the BI-CAR06 can still maintain a 50% cell killing rate against MOLP8 cells in the third run of killing (R3-1d) and a 40% cell killing rate against RPMI 8226 cells in the third run of killing (R3-1d).

### Example 13. NKG2D-F CAR Constructed by Humanized Antibodies

### 13.1 Construction of NKG2D-F CAR

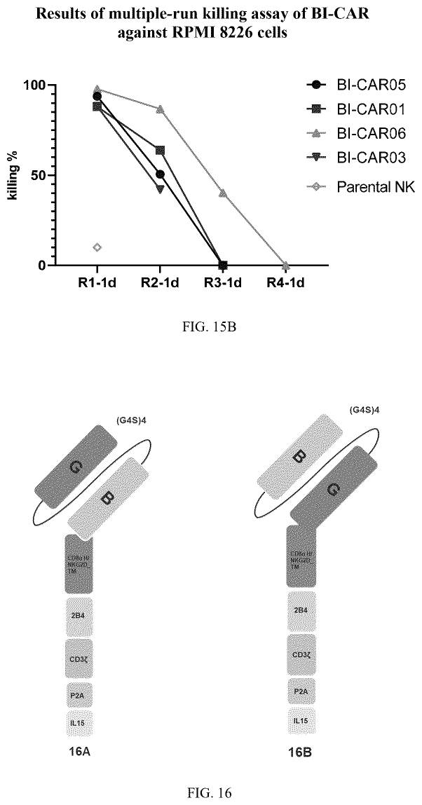

Dual-target chimeric antigen receptors targeting BCMA and GPRC5D containing CD8a signal peptide (SP), anti-GPRC5D antibody and anti-BCMA, CD8a hinge region, NKG2D-F transmembrane region (NKG2D-TM1), 2B4 co-stimulatory domain, and CD35, and IL15 linked by self-cleaving peptide P2A were designed and constructed. The structures of BI-CAR18 and BI-CAR20 are shown in FIG. 16A, and the structures of BI-CAR19 and BI-CAR21 are shown in FIG. 16B. The sequences are shown in Table 21.

TABLE 21
Amino acid sequences of NKG2D-F CAR

| Sequence | Sequence No. | Amino acid sequence |
|---|---|---|
| BI-CAR18 | SEQ ID NO: 127 | MALPVTALLLPLALLLHAARPEVQLVESGGGLVQPGGSLRLSCAASGIL |
| | | VSAYVMAWYRQAPGKQRVLVSRLSTDGRTTYADSVKGRFTISKDNAK |
| | | NTLYLQMNSLRAEDTAVYYCYAERSRGHWGQGTMVTVSSGGGGSGG |
| | | GGSGGGGSGGGGSEVQLVESGGGLVQPGGSLRLSCAASESISSIHIMAW |
| | | YRQAPGKQRELVAGIRNDGSTVYVDSVKGRFTISRDNAKNSVYLQMN |
| | | SLRAEDTAVYYCNADQGFGSYSEWERRSRWGQGTTVTVSSTTTPAPRP |
| | | PTPAPTIASQPLSLRPEACRPAAGGAVHTRGLDFACDSPFFFCCFIAVAM |
| | | GIRFIIMVAIWSAVFLNSWRRKRKEKQSETSPKEFLTIYEDVKDLKTRRN |
| | | HEQEQTFPGGGSTIYSMIQSQSSAPTSQEPAYTLYSLIQPSRKSGSRKRN |
| | | HSPSFNSTIYEVIGKSQPKAQNPARLSRKELENFDVYSRVKFSRSADAPA |
| | | YQQGQNQLYNELNLGRREEYDVLDKRRGRDPEMGGKPRRKNPQEGL |
| | | YNELQKDKMAEAYSEIGMKGERRRGKGHDGLYQGLSTATKDTYDAL |
| | | HMQALPPRGSGATNFSLLKQAGDVEENPGPMRISKPHLRSISIQCYLCL |
| | | LLNSHFLTEAGIHVFILGCFSAGLPKTEANWVNVISDLKKIEDLIQSMHI |
| | | DATLYTESDVHPSCKVTAMKCFLLELQVISLESGDASIHDTVENLIILAN |
| | | NSLSSNGNVTESGCKECEELEEKNIKEFLQSFVHIVQMFINTS |
| BI-CAR19 | SEQ ID NO: 128 | MALPVTALLLPLALLLHAARPEVQLVESGGGLVQPGGSLRLSCAASESI |
| | | SSIHIMAWYRQAPGKQRELVAGIRNDGSTVYVDSVKGRFTISRDNAKN |
| | | SVYLQMNSLRAEDTAVYYCNADQGFGSYSEWERRSRWGQGTTVTVSS |
| | | GGGGSGGGGSGGGGSGGGGSEVQLVESGGGLVQPGGSLRLSCAASGIL |
| | | VSAYVMAWYRQAPGKQRVLVSRLSTDGRTTYADSVKGRFTISKDNAK |
| | | NTLYLQMNSLRAEDTAVYYCYAERSRGHWGQGTMVTVSSTTTPAPRP |
| | | PTPAPTIASQPLSLRPEACRPAAGGAVHTRGLDFACDSPFFFCCFIAVAM |
| | | GIRFIIMVAIWSAVFLNSWRRKRKEKQSETSPKEFLTIYEDVKDLKTRRN |
| | | HEQEQTFPGGGSTIYSMIQSQSSAPTSQEPAYTLYSLIQPSRKSGSRKRN |
| | | HSPSFNSTIYEVIGKSQPKAQNPARLSRKELENFDVYSRVKFSRSADAPA |
| | | YQQGQNQLYNELNLGRREEYDVLDKRRGRDPEMGGKPRRKNPQEGL |
| | | YNELQKDKMAEAYSEIGMKGERRRGKGHDGLYQGLSTATKDTYDAL |
| | | HMQALPPRGSGATNFSLLKQAGDVEENPGPMRISKPHLRSISIQCYLCL |
| | | LLNSHFLTEAGIHVFILGCFSAGLPKTEANWVNVISDLKKIEDLIQSMHI |
| | | DATLYTESDVHPSCKVTAMKCFLLELQVISLESGDASIHDTVENLIILAN |
| | | NSLSSNGNVTESGCKECEELEEKNIKEFLQSFVHIVQMFINTS |
| BI-CAR20 | SEQ ID NO: 129 | MALPVTALLLPLALLLHAARPEVQLVESGGGLVQPGGSLRLSCAASGF |
| | | TFSSAWMYWFRQAPGKGLEWVSHIDPRGGSTYYADSVKGRFTISRDN |
| | | SKNTLYLQMNSLRAEDTAVYYCAKDLRGLGRGQGTMVTVSSGGGGS |
| | | GGGGSGGGGSGGGGSEVQLVESGGGLVQPGGSLRLSCAASESISSIHIM |
| | | AWYRQAPGKQRELVAGIRNDGSTVYVDSVKGRFTISRDNAKNSVYLQ |
| | | MNSLRAEDTAVYYCNADQGFGSYSEWERRSRWGQGTTVTVSSTTTPA |
| | | PRPPTPAPTIASQPLSLRPEACRPAAGGAVHTRGLDFACDSPFFFCCFIAV |
| | | AMGIRFIIMVAIWSAVFLNSWRRKRKEKQSETSPKEFLTIYEDVKDLKT |
| | | RRNHEQEQTFPGGGSTIYSMIQSQSSAPTSQEPAYTLYSLIQPSRKSGSR |
| | | KRNHSPSFNSTIYEVIGKSQPKAQNPARLSRKELENFDVYSRVKFSRSA |
| | | DAPAYQQGQNQLYNELNLGRREEYDVLDKRRGRDPEMGGKPRRKNP |
| | | QEGLYNELQKDKMAEAYSEIGMKGERRRGKGHDGLYQGLSTATKDTY |
| | | DALHMQALPPRGSGATNFSLLKQAGDVEENPGPMRISKPHLRSISIQCY |
| | | LCLLLNSHFLTEAGIHVFILGCFSAGLPKTEANWVNVISDLKKIEDLIQS |
| | | MHIDATLYTESDVHPSCKVTAMKCFLLELQVISLESGDASIHDTVENLII |
| | | LANNSLSSNGNVTESGCKECEELEEKNIKEFLQSFVHIVQMFINTS |
| BI-CAR21 | SEQ ID NO: 130 | MALPVTALLLPLALLLHAARPEVQLVESGGGLVQPGGSLRLSCAASESI |
| | | SSIHIMAWYRQAPGKQRELVAGIRNDGSTVYVDSVKGRFTISRDNAKN |
| | | SVYLQMNSLRAEDTAVYYCNADQGFGSYSEWERRSRWGQGTTVTVSS |
| | | GGGGSGGGGSGGGGSGGGGSEVQLVESGGGLVQPGGSLRLSCAASGF |
| | | TFSSAWMYWFRQAPGKGLEWVSHIDPRGGSTYYADSVKGRFTISRDN |
| | | SKNTLYLQMNSLRAEDTAVYYCAKDLRGLGRGQGTMVTVSSTTTPAP |
| | | RPPTPAPTIASQPLSLRPEACRPAAGGAVHTRGLDFACDSPFFFCCFIAVA |
| | | MGIRFIIMVAIWSAVFLNSWRRKRKEKQSETSPKEFLTIYEDVKDLKTR |
| | | RNHEQEQTFPGGGSTIYSMIQSQSSAPTSQEPAYTLYSLIQPSRKSGSRK |
| | | RNHSPSFNSTIYEVIGKSQPKAQNPARLSRKELENFDVYSRVKFSRSAD |
| | | APAYQQGQNQLYNELNLGRREEYDVLDKRRGRDPEMGGKPRRKNPQ |
| | | EGLYNELQKDKMAEAYSEIGMKGERRRGKGHDGLYQGLSTATKDTYD |
| | | ALHMQALPPRGSGATNFSLLKQAGDVEENPGPMRISKPHLRSISIQCYL |
| | | CLLLNSHFLTEAGIHVFILGCFSAGLPKTEANWVNVISDLKKIEDLIQSM |
| | | HIDATLYTESDVHPSCKVTAMKCFLLELQVISLESGDASIHDTVENLIIL |
| | | ANNSLSSNGNVTESGCKECEELEEKNIKEFLQSFVHIVQMFINTS |

### 13.2 Detection of NKG2D-F CAR Expression

The expression of BI CARs 18-21 was detected by referring to the method in Example 5.1. The results, as shown in Table 22, indicate that the expression of BCMA CAR and GPRC5D CAR could be detected after NK cell infection at MOI=5; the positive rates of BCMA CARs were relatively higher and reached 60% or higher; the positive rates of GPRC5D CARs were low with the descending expression rate order being BI-CAR06 (69.60%)>BI-CAR21 (54.20%)>BI-CAR20 (54.00%)>BI-CAR18 (32.30%)>BI-CAR19 (13.80%).

TABLE 22
Results of NKG2D-F CAR expression rate

| BI-CAR | BCMA-CAR expression | GPRC5D-CAR expression |
|---|---|---|
| BI-CAR18 | 66.10% | 32.30% |
| BI-CAR19 | 86.00% | 13.80% |
| BI-CAR20 | 83.00% | 54.00% |
| BI-CAR21 | 90.00% | 54.20% |
| BI-CAR06 | 95.00% | 69.60% |
| NK | 10.00% | 1.57% |

### 13.3 Detection of NKG2D-F CAR-NK Cell Proliferation

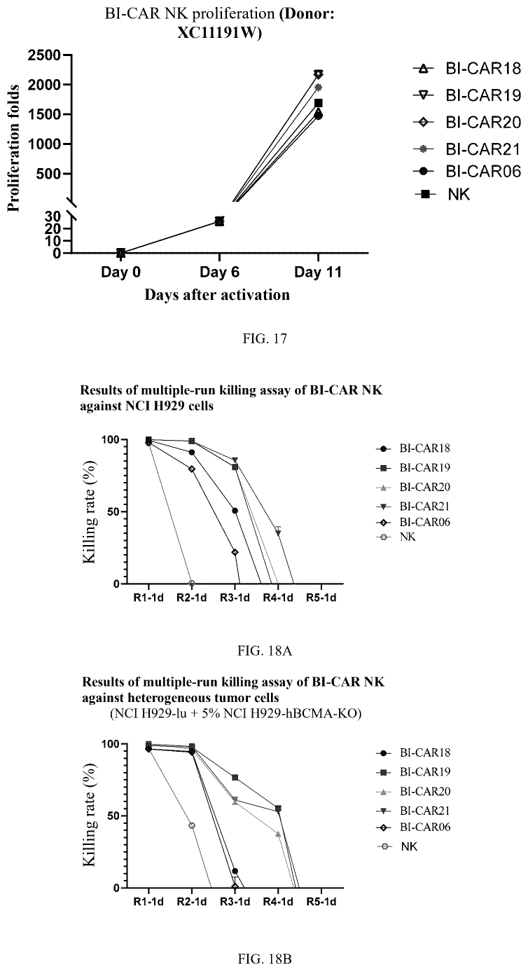

The proliferation of BI-CAR NK cells was detected by AO/PI counting, and the results are shown in FIG. 17 and Table 23. The BI-CAR NK cells were expanded by about 1473-2166 folds on day 11 after the activation, and the descending proliferation rate order was BI-CAR19 (2166 folds)=BI-CAR20 (2166 folds)>BI-CAR21 (1950 folds)>parental NK (1690 folds)>BI-CAR18 (1538 folds)>BI-CAR06 (1473 folds). As can be seen from the results described above, the proliferation rates of BI-CAR18, BI-CAR19, BI-CAR20, and BI-CAR21 cells constructed from the humanized antibodies were superior to that of the BI-CAR06.

TABLE 23
Detection of NKG2D-F CAR-NK cell proliferation

| BI-CAR | Proliferation folds |
|---|---|
| BI-CAR18 | 1538.33 |
| BI-CAR19 | 2166.67 |
| BI-CAR20 | 2166.67 |
| BI-CAR21 | 1950.00 |
| BI-CAR06 | 1473.33 |
| NK | 1690.00 |

### 13.4 Detection of Multiple-Run Killing Effects of NKG2D-F CAR-NK

On day 6 after the retroviral infection of NK cells, the multiple-run in vitro killing effects of BI-CAR18, BI-CAR19, BI-CAR20, BI-CAR21, and BI-CAR06 on NCI H929 cells, RPMI 8226 cells, MOLP8 cells, NCI H929-hBCMA-KO cells, and NCI H929+20% (or 10% or 5%) H929-hBCMA-KO heterogeneous tumor cells. The multiple-run in vitro killing effects on the cells described above were determined by referring to the method in Example 7.

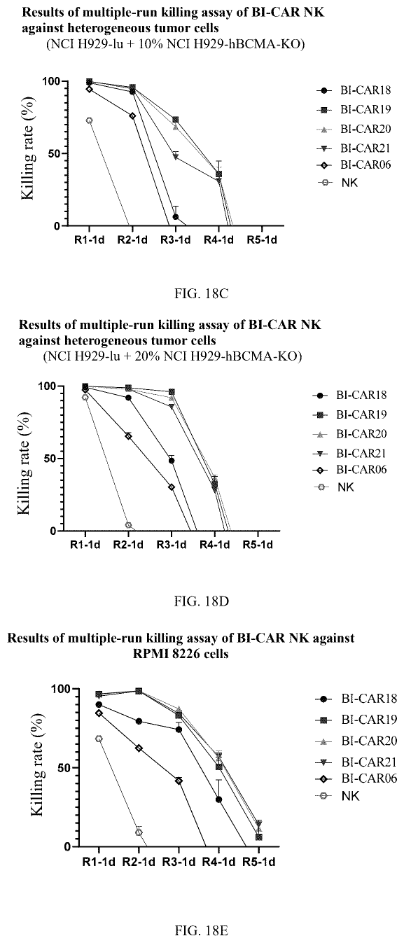

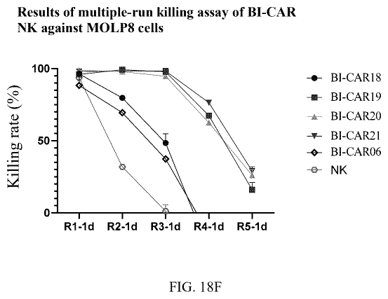

The results of multiple-run killing assay of BI-CAR NK cells, as shown in FIGS. 18A-18F, indicate that the multiple-run killing effects of the BI-CAR19, BI-CAR20, and BI-CAR21 were superior to those of the BI-CAR18 and BI-CAR06. The BI-CAR21 had the best killing effect on NCI H929 cells, and could still maintain a cell killing rate of 30% in the fourth run of killing (R4-1d); the BI-CAR19, BI-CAR20, and BI-CAR21 had the best killing effect on NCI H929+20% (or 10% or 5%) H929-hBCMA-KO heterogeneous tumor cells, RPMI 8226 cells, and MOLP8 cells, and could still maintain a cell killing rate of 30% or higher in the fourth run of killing (R4-1d) against NCI H929+20% (or 10% or 5%) H929-hBCMA-KO heterogeneous tumor cells and a cell killing rate of about 10% in the fifth run of killing (R5-1d) against RPMI 8226 cells and MOLP8 cells.

1. A nucleic acid molecule, comprising a first nucleic acid sequence encoding a chimeric antigen receptor (CAR), wherein the CAR comprises: an extracellular domain comprising an antigen-binding region, an NKG2D transmembrane region linked to the extracellular domain, and an intracellular domain linked to the transmembrane region; the NKG2D transmembrane region comprises a sequence having at least 80% identity to or at most 9 mutations compared with SEQ ID NO: 48.

2. The nucleic acid molecule according to claim 1, wherein the antigen-binding region has at least one of the properties of groups (1)-(4):
(1) the antigen-binding region is selected from an antibody or a fragment thereof or a ligand binding to the antigen, e.g., scFv, VHH, Fab, F(ab)′₂, or ligand;
(2) the antigen-binding region binds to or does not bind to NKG2DL;
(3) the antigen is selected from one or more of the following group: BCMA, GPRC5D, CLDN18.2, ROR1, CD19, CD33, CD5, CD70, HER2, IL13Rα2, GCC, or GPC3;
(4) the antigen-binding region binds to at least two different antigens, binds to at least two different epitopes of the same antigen, or binds to two or more identical epitopes of the same antigen.

3. The nucleic acid molecule according to claim 1, wherein the antigen-binding region specifically binds to BCMA, and comprises:
(1) a VHH, comprising CDRs 1-3 respectively comprising:
the sequences set forth in SEQ ID NO: 9, SEQ ID NO: 10, and SEQ ID NO: 11; or
the sequences set forth in SEQ ID NO: 12, SEQ ID NO: 13, and SEQ ID NO: 11; or
the sequences set forth in SEQ ID NO: 14, SEQ ID NO: 15, and SEQ ID NO: 16;
(2) a VHH, comprising a sequence having at least 80% identity to or at most 25 mutations compared with SEQ ID NO: 1 or 3;
(3) a VHH, comprising CDRs 1-3 respectively comprising:
the sequences set forth in SEQ ID NO: 17, SEQ ID NO: 18, and SEQ ID NO: 19; or
the sequences set forth in SEQ ID NO: 17, SEQ ID NO: 25, and SEQ ID NO: 19; or
the sequences set forth in SEQ ID NO: 20, SEQ ID NO: 21, and SEQ ID NO: 19; or
the sequences set forth in SEQ ID NO: 22, SEQ ID NO: 23, and SEQ ID NO: 24;
(4) a sequence having at least 80% identity to or at most 25 mutations compared with SEQ ID NO: 2 or 4; or
(5) VHH1-linker peptide-VHH2, wherein the VHH1 comprises the sequence set forth in group (1) or group (2), and the VHH2 comprises the sequence set forth in group (3) or group (4); or
(6) VHH1-linker peptide-VHH2, wherein the VHH1 comprises the sequence set forth in group (3) or group (4), and the VHH2 comprises the sequence set forth in group (1) or group (2); or
(7) VHH1-linker peptide-VHH2, comprising a sequence having at least 80% identity to or at most 50 mutations compared with SEQ ID NO: 63.

4. The nucleic acid molecule according to claim 1, wherein the antigen-binding region specifically binds to ROR1, and comprises:
(1) an antibody or a fragment thereof, comprising HCDRs 1-3 and LCDRs 1-3, wherein the HCDRs 1-3 respectively comprise:
the sequences set forth in SEQ ID NO: 30, SEQ ID NO: 31, and SEQ ID NO: 32; or
the sequences set forth in SEQ ID NO: 33, SEQ ID NO: 34, and SEQ ID NO: 32; or
the sequences set forth in SEQ ID NO: 35, SEQ ID NO: 36, and SEQ ID NO: 37;
and the LCDRs 1-3 respectively comprise:
the sequences set forth in SEQ ID NO: 38, SEQ ID NO: 39, and SEQ ID NO: 40; or
the sequences set forth in SEQ ID NO: 38, SEQ ID NO: 39, and SEQ ID NO: 40; or
the sequences set forth in SEQ ID NO: 41, SEQ ID NO: 42, and SEQ ID NO: 40;
(2) an antibody or a fragment thereof, comprising a heavy chain variable region having at least 80% identity to or at most 25 mutations compared with SEQ ID NO: 26 and a light chain variable region having at least 80% identity to or at most 25 mutations compared with SEQ ID NO: 27;
(3) an scFv, comprising the sequence set forth in group (1) or group (2), and optionally, comprising a light chain variable region having at least 80% identity to or at most 50 mutations compared with SEQ ID NO: 28.

5. The nucleic acid molecule according to claim 1, wherein the antigen-binding region specifically binds to GPRC5D, and comprises:
(1) an antibody or a fragment thereof, comprising HCDRs 1-3 and LCDRs 1-3, wherein the HCDRs 1-3 respectively comprise:
the sequences set forth in SEQ ID NO: 87, SEQ ID NO: 88, and SEQ ID NO: 89; or
the sequences set forth in SEQ ID NO: 90, SEQ ID NO: 91, and SEQ ID NO: 92; or
the sequences set forth in SEQ ID NO: 96, SEQ ID NO: 97, and SEQ ID NO: 98;
and the LCDRs 1-3 respectively comprise:
the sequences set forth in SEQ ID NO: 84, SEQ ID NO: 85, and SEQ ID NO: 86; or
the sequences set forth in SEQ ID NO: 93, SEQ ID NO: 94, and SEQ ID NO: 95;
(2) an antibody or a fragment thereof, comprising HCDRs 1-3, wherein the HCDRs 1-3 respectively comprise:
the sequences set forth in SEQ ID NOs: 107-118;
(3) an antibody or a fragment thereof, comprising a heavy chain variable region having at least 80% identity to or at most 25 mutations compared with SEQ ID NO: 78, 80, 83, or 131 and a light chain variable region having at least 80% identity to or at most 25 mutations compared with SEQ ID NO: 77, 79, 81, or 82;
(4) an antibody or a fragment thereof, comprising a heavy chain variable region having at least 80% identity to or at most 25 mutations compared with SEQ ID NOs: 99-106.

6. The nucleic acid molecule according to claim 1, wherein the extracellular domain further comprises a hinge region; the hinge region is selected from one or more of the following group: a CD8α hinge region, a 2B4 hinge region, a CD28 hinge region, an IgG1 hinge region, an IgD hinge, an IgG4 hinge region, a GS hinge, a KIR2DS2 hinge, a KIR hinge, an NCR hinge, a SLAMF hinge, a CD16 hinge, a CD64 hinge, or an LY49 hinge; optionally, the hinge region is selected from a CD8α hinge region; e.g., the hinge region comprises a sequence having at least 80% identity to or at most 10 mutations compared with SEQ ID NO: 45, and optionally, wherein the extracellular domain further comprises a signal peptide; the signal peptide is selected from one or more of the following group: a CD8α signal peptide, an IgG1 heavy chain signal peptide, and a GM-CSFR2 signal peptide; optionally, the signal peptide is a CD8α signal peptide; e.g., the signal peptide comprises a sequence having at least 80% identity to or at most 5 mutations compared with SEQ ID NO: 43.

7. (canceled)

8. The nucleic acid molecule according to claim 1, wherein the intracellular domain comprises an intracellular signaling domain and/or a co-stimulatory domain, wherein
optionally, the intracellular signaling domain is CD32; e.g., the intracellular signaling domain comprises a sequence having at least 80% identity to or at most 35 mutations compared with SEQ ID NO: 55; and
optionally, the co-stimulatory domain is selected from one or more of the following group: a CD27 co-stimulatory domain, a 4-1BB co-stimulatory domain, an OX40 co-stimulatory domain, a 2B4 co-stimulatory domain, a CD28 co-stimulatory domain, an ICOS co-stimulatory domain, a DAP10 co-stimulatory domain, or a DAP12 co-stimulatory domain; optionally, the co-stimulatory domain is a 2B4 co-stimulatory domain; e.g., the co-stimulatory domain comprises a sequence having at least 80% identity to or at most 25 mutations compared with SEQ ID NO: 53; and
optionally, wherein the CAR comprises: the CD8α hinge region, the NKG2D transmembrane region, the 2B4 co-stimulatory domain, and a CD3ζ intracellular signaling domain; e.g., the CAR comprises a sequence having at least 80% identity to or at most 50 mutations compared with SEQ ID NO: 58.

9. (canceled)

10. The nucleic acid molecule according to claim 1, further comprising a second nucleic acid sequence encoding IL15, wherein optionally the IL15 is selected from soluble IL15, membrane-binding IL15, or a complex of IL15 with a receptor thereof or a receptor fragment, and optionally the IL15 comprises a sequence having at least 80% identity to or at most 35 mutations compared with SEQ ID NO: 57;
optionally, the first nucleic acid sequence and the second nucleic acid sequence are linked via an IRES or a sequence encoding a self-cleaving peptide; the self-cleaving peptide is selected from a 2A peptide, e.g., P2A, T2A, F2A, or E2A; optionally the self-cleaving peptide is a P2A, e.g., comprising a sequence having at least 80% identity to or at most 5 mutations compared with SEQ ID NO: 56;
optionally, the nucleic acid encodes an amino acid sequence having at least 85% identity to or at most 100 mutations compared with SEQ ID NO: 59.

11. The nucleic acid molecule according to claim 1, comprising a nucleic acid sequence encoding a sequence having at least 80% identity to or at most 150 mutations compared with SEQ ID NO: 65 or SEQ ID NO: 70; and optionally, wherein the nucleic acid is a DNA or an RNA, wherein the RNA is preferably an mRNA.

12. (canceled)

13. A nucleic acid molecule, comprising a first nucleic acid sequence encoding a CAR targeting ROR1, wherein the CAR comprises an extracellular domain comprising an antigen-binding region, a transmembrane region linked to the extracellular domain, and an intracellular domain linked to the transmembrane region; the antigen-binding region comprises:
(1) an antibody or a fragment thereof, comprising HCDRs 1-3 and LCDRs 1-3, wherein the HCDRs 1-3 respectively comprise:
the sequences set forth in SEQ ID NO: 30, SEQ ID NO: 31, and SEQ ID NO: 32; or
the sequences set forth in SEQ ID NO: 33, SEQ ID NO: 34, and SEQ ID NO: 32; or
the sequences set forth in SEQ ID NO: 35, SEQ ID NO: 36, and SEQ ID NO: 37;
and the LCDRs 1-3 respectively comprise:
the sequences set forth in SEQ ID NO: 38, SEQ ID NO: 39, and SEQ ID NO: 40; or
the sequences set forth in SEQ ID NO: 38, SEQ ID NO: 39, and SEQ ID NO: 40; or
the sequences set forth in SEQ ID NO: 41, SEQ ID NO: 42, and SEQ ID NO: 40;
(2) an antibody or a fragment thereof, comprising a heavy chain variable region having at least 80% identity to or at most 25 mutations compared with SEQ ID NO: 26 and a light chain variable region having at least 80% identity to or at most 25 mutations compared with SEQ ID NO: 27;
(3) an scFv, comprising the sequence set forth in group (1) or group (2), and optionally, comprising a light chain variable region having at least 80% identity to or at most 50 mutations compared with SEQ ID NO: 28.

14. The nucleic acid molecule according to claim 13, wherein the CAR comprises: a CD8α hinge region, an NKG2D transmembrane region, a 2B4 co-stimulatory domain, and a CD32 intracellular signaling domain; e.g., the CAR comprises a sequence having at least 80% identity to or at most 50 mutations compared with SEQ ID NO: 58; and optionally, wherein the nucleic acid molecule comprising a nucleic acid sequence encoding a sequence having at least 80% identity to or at most 150 mutations compared with SEQ ID NO: 65 or SEQ ID NO: 70.

15. The nucleic acid molecule according to claim 13, further comprising a second nucleic acid sequence encoding IL15, wherein optionally the IL15 is selected from soluble IL15, membrane-binding IL15, or a complex of IL15 with a receptor thereof or a receptor fragment, and optionally the IL15 comprises a sequence having at least 80% identity to or at most 35 mutations compared with SEQ ID NO: 57;
optionally, the first nucleic acid sequence and the second nucleic acid sequence are linked via an IRES or a sequence encoding a self-cleaving peptide; the self-cleaving peptide is selected from a 2A peptide, e.g., P2A, T2A, F2A, or E2A; optionally the self-cleaving peptide is a P2A, e.g., comprising a sequence having at least 80% identity to or at most 5 mutations compared with SEQ ID NO: 56;
optionally, the nucleic acid encodes an amino acid sequence having at least 85% identity to or at most 100 mutations compared with SEQ ID NO: 59.

16. (canceled)

17. A CAR, encoded by the nucleic acid molecule according to claim 1.

18. A vector, comprising the nucleic acid molecule according to claim 1.

19. An immune effector cell, comprising the nucleic acid molecule according to claim 1, the CAR encoded by the nucleic acid molecule, or the vector comprising the nucleic acid molecule.

20. The immune effector cell according to claim 19, wherein the immune effector cell is an NK cell selected from an NK cell differentiated from an iPSC, an NK cell derived from peripheral blood or umbilical cord blood, or an NK92 cell.

21. A method for preparing the immune effector cell according to claim 19, comprising: providing an immune effector cell, and introducing the nucleic acid molecule into the immune effector cell.

22. A product prepared by the method according to claim 21.

23. A pharmaceutical composition, comprising the nucleic acid molecule according to claim 1, the CAR encoded by the nucleic acid molecule, the vector comprising the nucleic acid molecule, or the immune effector cell comprising the nucleic acid molecule, the CAR or the vector, and a pharmaceutically acceptable carrier.

24.-25. (canceled)

26. A method for treating a cancer or tumor, comprising: administering to a subject in need an effective amount of the nucleic acid molecule according to claim 1, the CAR encoded by the nucleic acid molecule, the vector comprising the nucleic acid molecule, the immune effector cell comprising the nucleic acid molecule, the CAR or the vector, or the pharmaceutical composition comprising the nucleic acid molecule, the CAR, the vector or the immune effector cell and a pharmaceutically acceptable carrier, wherein the cancer or tumor is selected from a hematologic tumor or a solid tumor; optionally, the hematological tumor is selected from myeloma, lymphoma, or leukemia, e.g., multiple myeloma, Hodgkin's lymphoma, non-Hodgkin's lymphoma, diffuse large B-cell lymphoma, follicular lymphoma, mantle cell lymphoma, acute myelogenous leukemia (AML), chronic lymphocytic leukemia (CLL), acute lymphocytic leukemia (ALL), chronic myelogenous leukemia (CML), or hairy cell leukemia (HCL); optionally, the solid tumor is selected from lung cancer, breast cancer, colorectal cancer, gastric cancer, pancreatic cancer, liver cancer, skin cancer, bladder cancer, ovarian cancer, uterine cancer, prostate cancer, or adrenal cancer.


SEQUENCE LISTING (131 sequences)

SEQ ID NO: 1 - protein, 126 aa, synthetic construct
QVQLVESGGGLVQPGGSLRLSCSGSGFTLDYYMIGWFRQAPGKEREGLSSISPADGSTYYADSVKGRFTISRDSAKNTVYLQMNNLKPEDTALYYCAAGNEATISWGFGPWEYDYWGQGTQVTVSS

SEQ ID NO: 2 - protein, 123 aa, synthetic construct
QVQLVESGGGLVQSRGSLRLSCAASESISSIHIMAWYRRAPGKQRELVAGIRNDGSTVYADSAKGRFTISRDNAKNTVYLQMNSLKPEDTAVYYCNADQGFGSYSEWERRSRWGQGTEVTVSS

SEQ ID NO: 3 - protein, 126 aa, synthetic construct
EVQLVESGGGLVQPGGSLRLSCSASGFTLDYYMIGWFRQAPGKEREGLSSISPADGSTYYADSVKGRFTISRDSSKNTVYLQMNSLRAEDTAVYYCAAGNEATISWGFGPWEYDYWGQGTMVTVSS

SEQ ID NO: 4 - protein, 123 aa, synthetic construct
EVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSS

SEQ ID NO: 5 - protein, 358 aa, synthetic construct
QVQLVESGGGLVQPGGSLRLSCSGSGFTLDYYMIGWFRQAPGKEREGLSSISPADGSTYYADSVKGRFTISRDSAKNTVYLQMNNLKPEDTALYYCAAGNEATISWGFGPWEYDYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 355 aa, synthetic construct
QVQLVESGGGLVQSRGSLRLSCAASESISSIHIMAWYRRAPGKQRELVAGIRNDGSTVYADSAKGRFTISRDNAKNTVYLQMNSLKPEDTAVYYCNADQGFGSYSEWERRSRWGQGTEVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 358 aa, synthetic construct
EVQLVESGGGLVQPGGSLRLSCSASGFTLDYYMIGWFRQAPGKEREGLSSISPADGSTYYADSVKGRFTISRDSSKNTVYLQMNSLRAEDTAVYYCAAGNEATISWGFGPWEYDYWGQGTMVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 355 aa, synthetic construct
EVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 5 aa, synthetic construct
YYMIG

SEQ ID NO: 10 - protein, 17 aa, synthetic construct
SISPADGSTYYADSVKG

SEQ ID NO: 11 - protein, 17 aa, synthetic construct
GNEATISWGFGPWEYDY

SEQ ID NO: 12 - protein, 7 aa, synthetic construct
GFTLDYY

SEQ ID NO: 13 - protein, 6 aa, synthetic construct
SPADGS

SEQ ID NO: 14 - protein, 8 aa, synthetic construct
GFTLDYYM

SEQ ID NO: 15 - protein, 8 aa, synthetic construct
ISPADGST

SEQ ID NO: 16 - protein, 19 aa, synthetic construct
AAGNEATISWGFGPWEYDY

SEQ ID NO: 17 - protein, 5 aa, synthetic construct
IHIMA

SEQ ID NO: 18 - protein, 16 aa, synthetic construct
GIRNDGSTVYADSAKG

SEQ ID NO: 19 - protein, 15 aa, synthetic construct
DQGFGSYSEWERRSR

SEQ ID NO: 20 - protein, 7 aa, synthetic construct
ESISSIH

SEQ ID NO: 21 - protein, 5 aa, synthetic construct
RNDGS

SEQ ID NO: 22 - protein, 8 aa, synthetic construct
ESISSIHI

SEQ ID NO: 23 - protein, 7 aa, synthetic construct
IRNDGST

SEQ ID NO: 24 - protein, 17 aa, synthetic construct
NADQGFGSYSEWERRSR

SEQ ID NO: 25 - protein, 16 aa, synthetic construct
GIRNDGSTVYVDSVKG

SEQ ID NO: 26 - protein, 118 aa, synthetic construct
DLQLQESGPGLVKPSQSLSLTCSVTGYSITSGYDWNWIRQFPGNKLEWMGYINYDGSNNYNPSLKNRISITRDTSKNQFFLRLSSVTTEDTATYFCARGILRGYFDYWGQGTTLTVSS

SEQ ID NO: 27 - protein, 111 aa, synthetic construct
EIVLTQSPGSLAVSLGQRATISCRASESVDNFGLSFMHWYQQKPGQPPKLLIYRASNLESGIPARFSGSGSRTDFTLTINPVETDDVATYYCQQSNKDPYTFGGGTKLEIK

SEQ ID NO: 28 - protein, 244 aa, synthetic construct
DLQLQESGPGLVKPSQSLSLTCSVTGYSITSGYDWNWIRQFPGNKLEWMGYINYDGSNNYNPSLKNRISITRDTSKNQFFLRLSSVTTEDTATYFCARGILRGYFDYWGQGTTLTVSSGGGGSGGGGSGGGGSEIVLTQSPGSLAVSLGQRATISCRASESVDNFGLSFMHWYQQKPGQPPKLLIYRASNLESGIPARFSGSGSRTDFTLTINPVETDDVATYYCQQSNKDPYTFGGGTKLEIK

SEQ ID NO: 29 - protein, 476 aa, synthetic construct
DLQLQESGPGLVKPSQSLSLTCSVTGYSITSGYDWNWIRQFPGNKLEWMGYINYDGSNNYNPSLKNRISITRDTSKNQFFLRLSSVTTEDTATYFCARGILRGYFDYWGQGTTLTVSSGGGGSGGGGSGGGGSEIVLTQSPGSLAVSLGQRATISCRASESVDNFGLSFMHWYQQKPGQPPKLLIYRASNLESGIPARFSGSGSRTDFTLTINPVETDDVATYYCQQSNKDPYTFGGGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 6 aa, synthetic construct
SGYDWN

SEQ ID NO: 31 - protein, 16 aa, synthetic construct
YINYDGSNNYNPSLKN

SEQ ID NO: 32 - protein, 9 aa, synthetic construct
GILRGYFDY

SEQ ID NO: 33 - protein, 8 aa, synthetic construct
GYSITSGY

SEQ ID NO: 34 - protein, 5 aa, synthetic construct
NYDGS

SEQ ID NO: 35 - protein, 9 aa, synthetic construct
GYSITSGYD

SEQ ID NO: 36 - protein, 7 aa, synthetic construct
INYDGSN

SEQ ID NO: 37 - protein, 11 aa, synthetic construct
ARGILRGYFDY

SEQ ID NO: 38 - protein, 15 aa, synthetic construct
RASESVDNFGLSFMH

SEQ ID NO: 39 - protein, 7 aa, synthetic construct
RASNLES

SEQ ID NO: 40 - protein, 9 aa, synthetic construct
QQSNKDPYT

SEQ ID NO: 41 - protein, 10 aa, synthetic construct
ESVDNFGLSF

SEQ ID NO: 42 - skipped

SEQ ID NO: 43 - protein, 21 aa, synthetic construct
MALPVTALLLPLALLLHAARP

SEQ ID NO: 44 - protein, 18 aa, synthetic construct
MLRLLLALNLFPSIQVTG

SEQ ID NO: 45 - protein, 45 aa, synthetic construct
TTTPAPRPPTPAPTIASQPLSLRPEACRPAAGGAVHTRGLDFACD

SEQ ID NO: 46 - protein, 14 aa, synthetic construct
QDCQNAHQEFRFWP

SEQ ID NO: 47 - protein, 38 aa, synthetic construct
EVMYPPPYLDNEKSNGTIIHVKGKHLCPSPLFPGPSKP

SEQ ID NO: 48 - protein, 31 aa, synthetic construct
SPFFFCCFIAVAMGIRFIIMVAIWSAVFLNS

SEQ ID NO: 49 - protein, 21 aa, synthetic construct
PFFFCCFIAVAMGIRFIIMVT

SEQ ID NO: 50 - protein, 20 aa, synthetic construct
PFFFCCFIAVAMGIRFIIMV

SEQ ID NO: 51 - protein, 21 aa, synthetic construct
FLVIIVILSALFLGTLACFCV

SEQ ID NO: 52 - protein, 27 aa, synthetic construct
FWVLVVVGGVLACYSLLVTVAFIIFWV

SEQ ID NO: 53 - protein, 120 aa, synthetic construct
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 54 - protein, 41 aa, synthetic construct
RSKRSRLLHSDYMNMTPRRPGPTRKHYQPYAPPRDFAAYRS

SEQ ID NO: 55 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 19 aa, synthetic construct
ATNFSLLKQAGDVEENPGP

SEQ ID NO: 57 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 58 - protein, 308 aa, synthetic construct
TTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 492 aa, synthetic construct
TTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 60 - protein, 126 aa, synthetic construct; identical to SEQ ID NO: 3

SEQ ID NO: 61 - protein, 123 aa, synthetic construct; identical to SEQ ID NO: 4

SEQ ID NO: 62 - protein, 5 aa, synthetic construct
GGGGS

SEQ ID NO: 63 - protein, 254 aa, synthetic construct
EVQLVESGGGLVQPGGSLRLSCSASGFTLDYYMIGWFRQAPGKEREGLSSISPADGSTYYADSVKGRFTISRDSSKNTVYLQMNSLRAEDTAVYYCAAGNEATISWGFGPWEYDYWGQGTMVTVSSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSS

SEQ ID NO: 64 - protein, 239 aa, synthetic construct
MVSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 65 - protein, 767 aa, synthetic construct
MALPVTALLLPLALLLHAARPEVQLVESGGGLVQPGGSLRLSCSASGFTLDYYMIGWFRQAPGKEREGLSSISPADGSTYYADSVKGRFTISRDSSKNTVYLQMNSLRAEDTAVYYCAAGNEATISWGFGPWEYDYWGQGTMVTVSSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 66 - protein, 757 aa, synthetic construct
MALPVTALLLPLALLLHAARPEVQLVESGGGLVQPGGSLRLSCSASGFTLDYYMIGWFRQAPGKEREGLSSISPADGSTYYADSVKGRFTISRDSSKNTVYLQMNSLRAEDTAVYYCAAGNEATISWGFGPWEYDYWGQGTMVTVSSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 67 - protein, 756 aa, synthetic construct
MALPVTALLLPLALLLHAARPEVQLVESGGGLVQPGGSLRLSCSASGFTLDYYMIGWFRQAPGKEREGLSSISPADGSTYYADSVKGRFTISRDSSKNTVYLQMNSLRAEDTAVYYCAAGNEATISWGFGPWEYDYWGQGTMVTVSSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDPFFFCCFIAVAMGIRFIIMVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 68 - protein, 614 aa, synthetic construct
MALPVTALLLPLALLLHAARPEVQLVESGGGLVQPGGSLRLSCSASGFTLDYYMIGWFRQAPGKEREGLSSISPADGSTYYADSVKGRFTISRDSSKNTVYLQMNSLRAEDTAVYYCAAGNEATISWGFGPWEYDYWGQGTMVTVSSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 69 - protein, 681 aa, synthetic construct
MALPVTALLLPLALLLHAARPEVQLVESGGGLVQPGGSLRLSCSASGFTLDYYMIGWFRQAPGKEREGLSSISPADGSTYYADSVKGRFTISRDSSKNTVYLQMNSLRAEDTAVYYCAAGNEATISWGFGPWEYDYWGQGTMVTVSSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 70 - protein, 757 aa, synthetic construct
MALPVTALLLPLALLLHAARPDLQLQESGPGLVKPSQSLSLTCSVTGYSITSGYDWNWIRQFPGNKLEWMGYINYDGSNNYNPSLKNRISITRDTSKNQFFLRLSSVTTEDTATYFCARGILRGYFDYWGQGTTLTVSSGGGGSGGGGSGGGGSEIVLTQSPGSLAVSLGQRATISCRASESVDNFGLSFMHWYQQKPGQPPKLLIYRASNLESGIPARFSGSGSRTDFTLTINPVETDDVATYYCQQSNKDPYTFGGGTKLEIKTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 71 - protein, 747 aa, synthetic construct
MALPVTALLLPLALLLHAARPDLQLQESGPGLVKPSQSLSLTCSVTGYSITSGYDWNWIRQFPGNKLEWMGYINYDGSNNYNPSLKNRISITRDTSKNQFFLRLSSVTTEDTATYFCARGILRGYFDYWGQGTTLTVSSGGGGSGGGGSGGGGSEIVLTQSPGSLAVSLGQRATISCRASESVDNFGLSFMHWYQQKPGQPPKLLIYRASNLESGIPARFSGSGSRTDFTLTINPVETDDVATYYCQQSNKDPYTFGGGTKLEIKTTTPAPRPPTPAPTIASQPLSLRPEACRPAAGGAVHTRGLDFACDPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 72 - protein, 746 aa, synthetic construct
MALPVTALLLPLALLLHAARPDLQLQESGPGLVKPSQSLSLTCSVTGYSITSGYDWNWIRQFPGNKLEWMGYINYDGSNNYNPSLKNRISITRDTSKNQFFLRLSSVTTEDTATYFCARGILRGYFDYWGQGTTLTVSSGGGGSGGGGSGGGGSEIVLTQSPGSLAVSLGQRATISCRASESVDNFGLSFMHWYQQKPGQPPKLLIYRASNLESGIPARFSGSGSRTDFTLTINPVETDDVATYYCQQSNKDPYTFGGGTKLEIKTTTPAPRPPTPAPTIASQPLSLRPEACRPAAGGAVHTRGLDFACDPFFFCCFIAVAMGIRFIIMVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 73 - protein, 604 aa, synthetic construct
MALPVTALLLPLALLLHAARPDLQLQESGPGLVKPSQSLSLTCSVTGYSITSGYDWNWIRQFPGNKLEWMGYINYDGSNNYNPSLKNRISITRDTSKNQFFLRLSSVTTEDTATYFCARGILRGYFDYWGQGTTLTVSSGGGGSGGGGSGGGGSEIVLTQSPGSLAVSLGQRATISCRASESVDNFGLSFMHWYQQKPGQPPKLLIYRASNLESGIPARFSGSGSRTDFTLTINPVETDDVATYYCQQSNKDPYTFGGGTKLEIKQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 74 - protein, 671 aa, synthetic construct
MALPVTALLLPLALLLHAARPDLQLQESGPGLVKPSQSLSLTCSVTGYSITSGYDWNWIRQFPGNKLEWMGYINYDGSNNYNPSLKNRISITRDTSKNQFFLRLSSVTTEDTATYFCARGILRGYFDYWGQGTTLTVSSGGGGSGGGGSGGGGSEIVLTQSPGSLAVSLGQRATISCRASESVDNFGLSFMHWYQQKPGQPPKLLIYRASNLESGIPARFSGSGSRTDFTLTINPVETDDVATYYCQQSNKDPYTFGGGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 75 - protein, 31 aa, synthetic construct
SNLFVASWIAVMIIFRIGMAVAIFCCFFFPS

SEQ ID NO: 76 - protein, 767 aa, synthetic construct
MALPVTALLLPLALLLHAARPEVQLVESGGGLVQPGGSLRLSCSASGFTLDYYMIGWFRQAPGKEREGLSSISPADGSTYYADSVKGRFTISRDSSKNTVYLQMNSLRAEDTAVYYCAAGNEATISWGFGPWEYDYWGQGTMVTVSSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 77 - protein, 107 aa, synthetic construct
DIVMTQSQKFMSTTVGDRVTITCKASQNVGTAVAWYQQKPGQSPKLLISSASNRYTGVPERFTGSGSGTDFTLTIRNMQSEDLADYFCQQYSSYPWTFGGGTELEIK

SEQ ID NO: 78 - protein, 118 aa, synthetic construct
QAYLQQSGAELVRPGASVKMSCKASGYTFTSYNMNWVKQTPRQGLEWIGAIYPGNGDTSYNQKFKGKATLTVDKSSSTAYMQLSSLTSEDSAVYFCARVRLRYAMDYWGQGTSVTVSS

SEQ ID NO: 79 - protein, 107 aa, synthetic construct
DIQMTQSPSSLSASVGDRVTITCKASQNVGTAVAWYQQKPGKSPKLLISSASNRYTGVPSRFSGSGSGTDFTLTISSLQPEDFATYYCQQYSSYPWTFGGGTKVEIK

SEQ ID NO: 80 - protein, 118 aa, synthetic construct
EAYLQQSGAEVKKPGASVKVSCKASGYTFTSYNMNWVRQAPGQRLEWMGAIYPGNADTSYNQKFKGRVTITVDKSASTAYMELSSLRSEDTAVYYCARVRLRYAMDYWGQGTTVTVSS

SEQ ID NO: 81 - protein, 107 aa, synthetic construct
DIVMTQSHKFMSTSVGDRVSITCKASQDVRTAVAWYQQKPGQSPKLLIYWASTRHTGVPDRFTGSRSGTDYTLTISSVQAEDLALYYCQQHYSTPLTFGAGTKLELK

SEQ ID NO: 82 - protein, 107 aa, synthetic construct
DIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIK

SEQ ID NO: 83 - protein, 120 aa, synthetic construct
QVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSS

SEQ ID NO: 84 - protein, 11 aa, synthetic construct
KASQNVGTAVA

SEQ ID NO: 85 - protein, 7 aa, synthetic construct
SASNRYT

SEQ ID NO: 86 - protein, 9 aa, synthetic construct
QQYSSYPWT

SEQ ID NO: 87 - protein, 5 aa, synthetic construct
SYNMN

SEQ ID NO: 88 - protein, 17 aa, synthetic construct
AIYPGNGDTSYNQKFKG

SEQ ID NO: 89 - protein, 9 aa, synthetic construct
VRLRYAMDY

SEQ ID NO: 90 - protein, 5 aa, synthetic construct
SYNMN

SEQ ID NO: 91 - protein, 17 aa, synthetic construct
AIYPGNADTSYNQKFKG

SEQ ID NO: 92 - protein, 9 aa, synthetic construct
VRLRYAMDY

SEQ ID NO: 93 - protein, 11 aa, synthetic construct
KASQDVRTAVA

SEQ ID NO: 94 - protein, 7 aa, synthetic construct
WASTRHT

SEQ ID NO: 95 - protein, 9 aa, synthetic construct
QQHYSTPLT

SEQ ID NO: 96 - protein, 5 aa, synthetic construct
SYGVN

SEQ ID NO: 97 - protein, 16 aa, synthetic construct
VIWSGGNTDYNAAFRS

SEQ ID NO: 98 - protein, 12 aa, synthetic construct
GQLGRPYWYFDV

SEQ ID NO: 99 - protein, 118 aa, synthetic construct
DVQLQASGGGSVQAGGSLRLSCAASGDTANCIGWFRQAPGKGREGVAAIYTGLGNTYYANSAKGRFTIAQDNAKNTVYLQMNSLKPEDSAMYYCAAGGILGRFCSRYFGQGTQVTVSS

SEQ ID NO: 100 - protein, 126 aa, synthetic construct
EVQLQASGGGSVQAGGSLRLSCVASGFPYSTSRVGWFRQAPGKEREGVATIVPGTGYTYYADSVKGRFTISQDSAKNTVYLQMNSLKPEDTAVYYCAAGDRRLLLNLLAPADYFYWGQGTQVTVSS

SEQ ID NO: 101 - protein, 118 aa, synthetic construct
EVQLVESGGGLVQPGGSLRLSCAASGDTANCIGWFRQAPGKGREGVAAIYTGLGNTYYANSAKGRFTISRDNAKNSVYLQMNSLRAEDTAVYYCAAGGILGRFCSRYFGQGTMVTVSS

SEQ ID NO: 102 - protein, 126 aa, synthetic construct
EVQLVESGGGLVQPGGSLRLSCAASGFPYSTSRVGWFRQAPGKEREGVATIVPGTGYTYYVDSVKGRFTISQDNAKNSVYLQMNSLRAEDTAVYYCAAGDRRLLLNLLAPADYFYWGQGTMVTVSS

SEQ ID NO: 103 - protein, 114 aa, synthetic construct
QVQLVESGGGLVQPGGSLRLSCAASGILVSAYVMAWYRQPPGKQRDLVARLSTDGRTTYADSVKGRFTISKDNAKSTLYLQMNTLKSEDTAVYYCYAERSRGHWGQGTQVTVSS

SEQ ID NO: 104 - protein, 115 aa, synthetic construct
QVQLVESGGGLVQPGGSLRLSCTASGFTFSSAWMYWFRQAPGKGLEWVSHIDPRGGSTYYADSVKGRFTISRDNAKNTLYLQMRSLKSEDTAVYYCAKDLRGLGRGQGTQVTVSS

SEQ ID NO: 105 - protein, 114 aa, synthetic construct
EVQLVESGGGLVQPGGSLRLSCAASGILVSAYVMAWYRQAPGKQRVLVSRLSTDGRTTYADSVKGRFTISKDNAKNTLYLQMNSLRAEDTAVYYCYAERSRGHWGQGTMVTVSS

SEQ ID NO: 106 - protein, 115 aa, synthetic construct
EVQLVESGGGLVQPGGSLRLSCAASGFTFSSAWMYWFRQAPGKGLEWVSHIDPRGGSTYYADSVKGRFTISRDNSKNTLYLQMNSLRAEDTAVYYCAKDLRGLGRGQGTMVTVSS

SEQ ID NO: 107 - skipped

SEQ ID NO: 108 - protein, 17 aa, synthetic construct
AIYTGLGNTYYANSAKG

SEQ ID NO: 109 - protein, 11 aa, synthetic construct
GGILGRFCSRY

SEQ ID NO: 110 - protein, 8 aa, synthetic construct
GFPYSTSR

SEQ ID NO: 111 - protein, 8 aa, synthetic construct
IVPGTGYT

SEQ ID NO: 112 - protein, 19 aa, synthetic construct
AAGDRRLLLNLLAPADYFY

SEQ ID NO: 113 - protein, 8 aa, synthetic construct
GILVSAYV

SEQ ID NO: 114 - protein, 7 aa, synthetic construct
LSTDGRT

SEQ ID NO: 115 - protein, 8 aa, synthetic construct
YAERSRGH

SEQ ID NO: 116 - protein, 5 aa, synthetic construct
SAWMY

SEQ ID NO: 117 - protein, 17 aa, synthetic construct
HIDPRGGSTYYADSVKG

SEQ ID NO: 118 - protein, 6 aa, synthetic construct
DLRGLG

SEQ ID NO: 119 - protein, 24 aa, synthetic construct
IYIWAPLAGTCGVLLLSLVITLYC

SEQ ID NO: 120 - protein, 42 aa, synthetic construct
KRGRKKLLYIFKQPFMRPVQTTQEEDGCSCRFPEEEEGGCEL

SEQ ID NO: 121 - protein, 813 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 122 - protein, 813 aa, synthetic construct
MALPVTALLLPLALLLHAARPEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSGGGGSGGGGSGGGGSGGGGSDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 123 - protein, 1061 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMALPVTALLLPLALLLHAARPEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 124 - protein, 1061 aa, synthetic construct
MALPVTALLLPLALLLHAARPEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 125 - protein, 811 aa, synthetic construct
MALPVTALLLPLALLLHAARPDIQMTQSPSSLSASVGDRVTITCKASQNVGTAVAWYQQKPGKSPKLLISSASNRYTGVPSRFSGSGSGTDFTLTISSLQPEDFATYYCQQYSSYPWTFGGGTKVEIKGGGGSGGGGSGGGGSEAYLQQSGAEVKKPGASVKVSCKASGYTFTSYNMNWVRQAPGQRLEWMGAIYPGNADTSYNQKFKGRVTITVDKSASTAYMELSSLRSEDTAVYYCARVRLRYAMDYWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 126 - protein, 898 aa, synthetic construct
MALPVTALLLPLALLLHAARPDIQMTQSPSSLSASVGDRVTITCKASQDVRTAVAWYQQKPGKSPKLLIYWASTRHTGVPDRFSGSGSGTDFTLTISSLQPEDVATYYCQQHYSTPLTFGQGTKLEIKGGGGSGGGGSGGGGSQVQLKESGPVLVKPTETLTLTCTVSGFSLTSYGVNWIRQPPGKALEWLAVIWSGGNTDYNAAFRSRLTISKDNSKSQVVLTMTNMDPVDTATYYCARGQLGRPYWYFDVWGQGTTVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 127 - protein, 770 aa, synthetic construct
MALPVTALLLPLALLLHAARPEVQLVESGGGLVQPGGSLRLSCAASGILVSAYVMAWYRQAPGKQRVLVSRLSTDGRTTYADSVKGRFTISKDNAKNTLYLQMNSLRAEDTAVYYCYAERSRGHWGQGTMVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 128 - protein, 770 aa, synthetic construct
MALPVTALLLPLALLLHAARPEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSGGGGSGGGGSGGGGSGGGGSEVQLVESGGGLVQPGGSLRLSCAASGILVSAYVMAWYRQAPGKQRVLVSRLSTDGRTTYADSVKGRFTISKDNAKNTLYLQMNSLRAEDTAVYYCYAERSRGHWGQGTMVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 129 - protein, 771 aa, synthetic construct
MALPVTALLLPLALLLHAARPEVQLVESGGGLVQPGGSLRLSCAASGFTFSSAWMYWFRQAPGKGLEWVSHIDPRGGSTYYADSVKGRFTISRDNSKNTLYLQMNSLRAEDTAVYYCAKDLRGLGRGQGTMVTVSSGGGGSGGGGSGGGGSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 130 - protein, 771 aa, synthetic construct
MALPVTALLLPLALLLHAARPEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSGGGGSGGGGSGGGGSGGGGSEVQLVESGGGLVQPGGSLRLSCAASGFTFSSAWMYWFRQAPGKGLEWVSHIDPRGGSTYYADSVKGRFTISRDNSKNTLYLQMNSLRAEDTAVYYCAKDLRGLGRGQGTMVTVSSTTTPAPRPPTPAPTIASQPLSLRPEACRPAAGGAVHTRGLDFACDSPFFFCCFIAVAMGIRFIIMVAIWSAVFLNSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 131 - protein, 120 aa, synthetic construct
QVQLKQSGPGLVQPSQSLSITCTVSGFSLTSYGVNWIRQSPGKGLEWLGVIWSGGNTDYNAAFRSRLSISKDNSKSQVFFKMNSLQADDTAIYYCARGQLGRPYWYFDVWGTGTSVTVSS